SEQUENCE LISTING (13 sequences)

SEQ ID NO: 1 - DNA, 585 bp, Mus sp.
gtcgacatgcatccagaagaccatttcctgagctttctggagagtgaatctgctcttaggggaaaagctcttccctttccttctccaaaaagctagaactgagctccaggaggctgactttctacagcatgaagcctacactgtgtttccttttcatcctcgtctcccttttcccactgatagtcccagggaacgcgcaatgctcctttgagtctttggtggatcaaaggatcaaggaagctctcagtcgtcaagagcctaagacgatctcctgcactagtgtcacgtcttctggcagactggcctcctgtcctgctgggatggttgtcactggatgtgcttgtggctatggctgtggatcgtgggatatccggaatggaaatacttgccactgccagtgctcagtcatggactgggcctctgcccgctgctgccgaatggcttaagaatgaggaggttgagaaaccaatttcaaaatgatgagcataatgaaaccacggtctcgaccaggaaacctgactcattgtcttcatattactaaataattcttcttgaataataaaggcagacctgtacctttgcatt

SEQ ID NO: 2 - protein, 105 aa, Mus sp.
MKPTLCFLFILVSLFPLIVPGNAQCSFESLVDQRIKEALSRQEPKTISCTSVTSSGRLASCPAGMVVTGCACGYGCGSWDIRNGNTCHCQCSVMDWASARCCRMA

SEQ ID NO: 3 - DNA, 603 bp, Homo sapiens
ggcacgaggccacgttgtcttctttccttcaccaccacccaggagctcagagatctaagctgctttccatcttttctcccagccccaggacactgactctgtacaggatggggccgtcctcttgcctccttctcatcctaatcccccttctccagctgatcaacccggggagtactcagtgttccttagactccgttatggataagaagatcaaggatgttctcaacagtctagagtacagtccctctcctataagcaagaagctctcgtgtgctagtgtcaaaagccaaggcagaccgtcctcctgccctgctgggatggctgtcactggctgtgcttgtggctatggctgtggttcgtgggatgttcagctggaaaccacctgccactgccagtgcagtgtggtggactggaccactgcccgctgctgccacctgacctgacagggaggaggctgagaactcagttttgtgaccatgacagtaatgaaaccagggtcccaaccaagaaatctaactcaaacgtcccacttcatttgttccattcctgattcttgggtaataaagacaaactttgtacctcaaaaaaaaaaaaaaaaaaaaa

SEQ ID NO: 4 - protein, 111 aa, Homo sapiens
MGPSSCLLLILIPLLQLINPGSTQCSLDSVMDKKIKDVLNSLEYSPSPISKKLSCASVKSQGRPSSCPAGMAVTGCACGYGCGSWDVQLETTCHCQCSVVDWTTARCCHLT

SEQ ID NO: 5 - DNA, 630 bp, Mus sp.
ggcacgagggggtacctaggtcagcaatcccatggcgtataaaagcatctcatctggccaggtcctggaacctttcctgagattctgccccaggatgccaactttgaataggatgaagactacaacttgttcccttctcatctgcatctccctgctccagctgatggtcccagtgaatactgatgagaccatagagattatcgtggagaataaggtcaaggaacttcttgccaatccagctaactatccctccactgtaacgaagactctctcttgcactagtgtcaagactatgaacagatgggcctcctgccctgctgggatgactgctactgggtgtgcttgtggctttgcctgtggatcttgggagatccagagtggagatacttgcaactgcctgtgcttactcgttgactggaccactgcccgctgctgccaactgtcctaagaatgaagaggtggagaacccagctttgatatgatgaatctaacaaaaactgcagtytcaatttggaaatctgactcatgtgcctttaaatgtgttcatattgcccatttaccctgcttcttgaaatgcttcttgaaaaataaagacaaatttgcatgtgaaaaaaaaaaaaaaaaaaaaaa

SEQ ID NO: 6 - protein, 111 aa, Mus sp.
MKTTTCSLLICISLLQLMVPVNTDETIEIIVENKVKELLANPANYPSTVTKTLSCTSVKTMNRWASCPAGMTATGCACGFACGSWEIQSGDTCNCLCLLVDWTTARCCQLS

SEQ ID NO: 7 - DNA, 537 bp, Rattus sp.
ggcacgaggggacactgactttcaacaggatgaagactgcaacctgttcccttctcatctgcgtcttccttctccagctgatggtcccagtgaatactgatggaaccttagacattattgggaagaaaaaggtcaaggaacttctagcccatcaagataactatccctctgctgtaaggaagaccctctcatgcactaatgtcaagtctatgagcaaatgggcctcctgccctgctgggatgactgctactggttgttcttgtggctttgcctgtggatcttgggaaatccagaatgaaaatatttgcaactgcctgtgcttaatcgttgactgggcctatgcccgctgctgccaactgtcctaagaatggagaggcacagaactcagctttgatatgatgagtctaacaaaactgcaatctcaacttggaaatctgactcatgcgcatttgatgtattcatattgcccattaaccccgcttcttgaaaaaataaagacaaatttacatcttaaaaaaaaaaaaaaaaaaaaaaaaa

SEQ ID NO: 8 - protein, 111 aa, Rattus sp.
MKTATCSLLICVFLLQLMVPVNTDGTLDIIGKKKVKELLAHQDNYPSAVRKTLSCTNVKSMSKWASCPAGMTATGCSCGFACGSWEIQNENICNCLCLIVDWAYARCCQLS

SEQ ID NO: 9 - DNA, 576 bp, Mus sp.
gtgggacagcgagctaatacccagaactgagttgtgtcctgctaagtcctctgccacgtacccacgggatgaagaacctttcatttcccctccttttccttttcttccttgtccctgaactgctgggctccagcatgccactgtgtcccatcgatgaagccatcgacaagaagatcaaacaagacttcaactccctgtttccaaatgcaataaagaacattggcttaaattgctggacagtctcctccagagggaagttggcctcctgcccagaaggcacagcagtcttgagctgctcctgtggctctgcctgtggctcgtgggacattcgtgaagaaaaagtgtgtcactgccagtgtgcaaggatagactggacagcagcccgctgctgtaagctgcaggtcgcttcctgatgtcggggaagtgagcgtggtttccagcacagccacccgttcctgtagctccagagatgtctgatgtcctccggtctctacaggcacctgcactcacgtgcgcgaatccacacacaagcacacatacttaaaaataaaacaaaacaggctggaaaaaaaaaaa

SEQ ID NO: 10 - protein, 114 aa, Mus sp.
MKNLSFPLLFLFFLVPELLGSSMPLCPIDEAIDKKIKQDFNSLFPNAIKNIGLNCWTVSSRGKLASCPEGTAVLSCSCGSACGSWDIREEKVCHCQCARIDWTAARCCKLQVAS

SEQ ID NO: 11 - DNA, 476 bp, Homo sapiens
  misc_feature (1)..(476): n can be either a, c, t, or g
gtgtgccggatttggttagctgagcccaccgagaggcgcctgcaggatgaaagctctctgtctcctcctcctccctgtcctggggctgttggtgtctagcaagaccctgtgctccatggaagaagccatcaatgagaggntccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagtgcnacagcgcgggcggaggcggctccaggtccggaggggttgcgggggagctggaaataaacctggagatgatgatgatgatgatgatgg

SEQ ID NO: 12 - protein, 108 aa, Homo sapiens
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 13 - protein, 138 aa, Mus sp.
MAYKSISSGQVLEPFLRFCPRMPTLNRMKTTTCSLLICISLLQLMVPVNTDETIEIIVENKVKELLANPANYPSTVTKTLSCTSVKTMNRWASCPAGMTATGCACGFACGSWEIQSGDTCNCLCLLVDWTTARCCQLS

(19) United States
(12) Patent Application Publication
(10) Pub. No.: US 2003/0138826 A1
(43) Pub. Date: Jul. 24, 2003

(54) Compositions, methods, and kits relating to resistin-like molecules

(75) Inventors: Mitchell A. Lazar, Gladwyne, PA (US); Gary D. Wu, Ardmore, PA (US)

Correspondence Address: MORGAN, LEWIS & BOCKIUS LLP, 1701 MARKET STREET, PHILADELPHIA, PA, 19103-2921, US

(73) Assignee: The Trustees of the University of Pennsylvania

(21) Appl. No.: 10/304,100

(22) Filed: Feb. 27, 2003

Related U.S. Application Data
(63) Continuation of application No. PCT/US01/18460, filed on Jun. 7, 2001.
(60) Provisional application No. 60/210,609, filed on Jun. 9, 2000.

(51) Int. Cl. C12Q 1/68; A01K 67/00; C07H 21/04; C12N 9/64; C12P 21/02; C12N 5/06

(52) U.S. Cl. 435/6; 435/69.1; 435/226; 435/320.1; 435/325; 536/23.2; 800/8

(57) ABSTRACT

The invention relates to novel nucleic acids encoding a mammalian resistin-like molecule (RELM), and proteins encoded thereby, whose expression is increased in certain diseases, disorders, or conditions, including, but not limited to, intestinal (e.g., colonic) tumors. The invention further relates to methods of treating and detecting irritable bowel disease, inflammatory bowel disease, familial adenomatous polyposis, diabetes, insulin resistance, obesity, Syndrome X, and glucose metabolism disorders, colon cancer, breast cancer, and tongue cancer, comprising modulating or detecting RELM expression and/or production and activity of RELM polypeptide, wherein RELM encompasses resistin-like molecule α and resistin-like molecule β.

### CROSS-REFERENCE TO RELATED APPLICATIONS

The present application is a continuation of International Patent Application No. PCT/US01/18460, filed on Jun. 7, 2001, which claims priority under 35 U.S.C. § 119(e), and to U.S. Provisional Application No. 60/210,609, filed Jun. 9, 2000.

### STATEMENT REGARDING FEDERALLY SPONSORED RESEARCH OR DEVELOPMENT

This invention was supported in part by funds from the U.S. Government (National Institutes of Health Grants No. DK49780 and No. DK49210) and the U.S. Government may therefore have certain rights in the invention.

### BACKGROUND OF THE INVENTION

Hormones are secreted molecules, often polypeptides, that have pleiotropic effects on a wide variety of tissues. Polypeptide hormones are secreted proteins that often are produced by specialized cells types in specific tissues and which usually, but not always, circulate in the bloodstream. When they do circulate in the bloodstream, their measurement is often a useful diagnosis, and their supplementation or antagonism is often a useful treatment modality. New classes of hormones have the potential to be involved in numerous diseases. The hormones could be deficient, inappropriately increased, or dysregulated leading to disease or discomfort.

Diseases of the colon are common. These include colon cancer, inflammatory bowel disease, and irritable bowel disease. The causes of these diseases, with the exception of rare familial colon cancer syndromes such as familial adenomatous polyposis (FAP), are unknown. These diseases cause tremendous morbidity and mortality in the U.S. and worldwide. Although these are disparate diseases, without exception the existing methods of diagnosis and, especially, of treatment are unsatisfactory.

FAP is a genetic disease associated with mutations in the adenomatous polyposis coli (APC) gene (Bodmer et al., 1999, Cytogenet. Cell Genet. 86:99-104). Other familial colon cancer syndromes are due to abnormalities in unknown genes (Peltomaki et al., 1999, Adv. Exp. Med. Biol. 470:95-98). Furthermore, most cases of colon cancer are sporadic. In these cases etiology is unknown and therapy is not mechanism-based despite the fact that this is one of the leading causes of cancer death worldwide.

Inflammatory bowel diseases include Crohn's disease and ulcerative colitis (Ghosh et al., 2000, BMJ 320:1119-1123). These are disabling diseases, which in the case of ulcerative colitis, are associated with colon cancer. The disease mechanism is unknown, diagnosis is difficult, and treatments are empiric.

Irritable bowel disease is not life threatening, but it is an extremely common and disabling condition, which is difficult to diagnose with certainty and whose etiology is completely unknown (Hammer & Talley, 1999, Am. J. Med. 107:5S-11S).

Presently, familial adenomatous polyposis, and irritable bowel disease are common causes of human morbidity and mortality for which there is no cure. Further, colon cancer, while treatable by resection if detected at an early stage, requires invasive diagnostics where the intestines must be visually inspected. Moreover, some portions of the intestine are not accessible to invasive inspection methods and abnormalities in those areas are not detectable by present methods.

Accordingly, there is a need for methods of diagnosing and treating colon cancer, familial adenomatous polyposis, irritable bowel disease, inflammatory bowel disease, intestinal tumors, breast cancer, tongue cancer, diabetes, insulin resistance, and obesity, and to identify genes associated with these diseases, disorders or conditions. The present invention satisfies these needs.

### BRIEF SUMMARY OF THE INVENTION

The invention includes an isolated nucleic acid encoding a mammalian resistin-like molecule, or a fragment thereof.

In one aspect, the isolated nucleic acid shares at least about 30% sequence identity with a nucleic acid encoding at least one of a mouse resistin-like molecule β (SEQ ID NO:1), a human resistin-like molecule β (SEQ ID NO:3), a mouse resistin-like molecule α (SEQ ID NO:5), and a rat resistin-like molecule α (SEQ ID NO:7).

The invention includes an isolated nucleic acid encoding a mammalian resistin-like molecule, wherein the amino acid sequence of the resistin-like molecule shares at least about 30% sequence identity with an amino acid sequence of at least one of SEQ ID NO:2, SEQ ID NO:4, SEQ ID NO:6, SEQ ID NO:8, and SEQ ID NO: 13.

The invention includes an isolated polypeptide comprising a mammalian resistin-like molecule.

The invention also includes an isolated polypeptide comprising a mammalian resistin-like molecule, wherein the mammalian resistin-like molecule shares at least about 30% sequence identity with at least one amino acid sequence selected from the group consisting of SEQ ID NO:2, SEQ ID NO:4, SEQ ID NO:6, SEQ ID NO:8, and SEQ ID NO:13.

The invention includes an isolated nucleic acid encoding a mammalian resistin-like molecule, or a fragment thereof, the nucleic acid further comprising a nucleic acid encoding a tag polypeptide covalently linked thereto.

In one aspect, the tag polypeptide is selected from the group consisting of a myc tag polypeptide, a glutathione-S-transferase tag polypeptide, a green fluorescent protein tag polypeptide, a myc-pyruvate kinase tag polypeptide, a His6 tag polypeptide, an influenza virus hemagglutinin tag polypeptide, a flag tag polypeptide, and a maltose binding protein tag polypeptide.

The invention includes an isolated nucleic acid encoding a mammalian resistin-like molecule, or a fragment thereof, the nucleic acid further comprising a nucleic acid specifying a promoter/regulatory sequence operably linked thereto.

The invention includes a vector comprising an isolated nucleic acid encoding a mammalian resistin-like molecule, or a fragment thereof.

In one aspect, the vector further comprises a nucleic acid specifying a promoter/regulatory sequence operably linked thereto.

The invention includes a recombinant cell comprising an isolated nucleic acid encoding a mammalian resistin-like molecule, or a fragment thereof.

The invention includes a recombinant cell comprising a vector wherein the comprises an isolated nucleic acid encoding a mammalian resistin-like molecule, or a fragment thereof

The invention includes an isolated nucleic acid complementary to an isolated nucleic acid encoding a mammalian resistin-like molecule, or a fragment thereof, the complementary nucleic acid being in an antisense orientation.

In one aspect, the nucleic acid shares at least about 30% identity with a nucleic acid complementary with a nucleic acid having the sequence of at least one of a mouse resistin-like molecule β (SEQ ID NO: 1), a human resistin-like molecule β (SEQ ID NO:3), a mouse resistin-like molecule α SEQ ID NO:5), and a rat resistin-like molecule α (SEQ ID NO:7).

The invention includes a recombinant cell comprising an isolated nucleic acid complementary to an isolated nucleic acid encoding a mammalian resistin-like molecule, or a fragment thereof, the complementary nucleic acid being in an antisense orientation.

The invention includes a vector comprising an isolated nucleic acid complementary to an isolated nucleic acid encoding a mammalian resistin-like molecule, or a fragment thereof, the complementary nucleic acid being in an antisense orientation.

The invention includes an antibody that specifically binds with a mammalian resistin-like molecule polypeptide, or a fragment thereof.

The invention includes an antibody that specifically binds with a mammalian resistin-like molecule polypeptide, or a fragment thereof, wherein the mammalian resistin-like molecule shares at least about 30% sequence identity with at least one amino acid sequence selected from the group consisting of SEQ ID NO:2, SEQ ID NO:4, SEQ ID NO:6, SEQ ID NO:8, and SEQ ID NO:13.

In one aspect, the antibody is selected from the group consisting of a polyclonal antibody, a monoclonal antibody, a humanized antibody, a chimeric antibody, and a synthetic antibody.

The invention also includes a composition comprising an antibody that specifically binds with a mammalian resistin-like molecule polypeptide, or a fragment thereof, and a pharmaceutically-acceptable carrier.

In one aspect, the mammalian resistin-like molecule polypeptide shares at least about 30% sequence identity with at least one amino acid sequence selected from the group consisting of SEQ ID NO:2, SEQ ID NO:4, SEQ ID NO:6, SEQ ID NO:8, and SEQ ID NO:13, or a fragment thereof.

The invention includes a composition comprising an isolated nucleic acid complementary to an isolated nucleic acid encoding a mammalian resistin-like molecule, or a fragment thereof, the complementary nucleic acid being in an antisense orientation, and a pharmaceutically-acceptable carrier.

In one aspect, the isolated nucleic acid encoding a mammalian resistin-like molecule, or a fragment thereof, shares at least about 30% sequence identity with a nucleic acid encoding at least one of a mouse resistin-like molecule β (SEQ ID NO: 1), a human resistin-like molecule β (SEQ ID NO:3), a mouse resistin-like molecule α (SEQ ID NO:5), and a rat resistin-like molecule α (SEQ ID NO:7).

The invention further includes a composition comprising an isolated nucleic acid encoding a mammalian resistin-like molecule, or a fragment thereof, and a pharmaceutically-acceptable carrier.

In one aspect, the isolated nucleic acid encoding a mammalian resistin-like molecule, or a fragment thereof, shares at least about 30% sequence identity with a nucleic acid encoding at least one of a mouse resistin-like molecule β (SEQ ID NO: 1), a human resistin-like molecule β (SEQ ID NO:3), a mouse resistin-like molecule α (SEQ ID NO:5), and a rat resistin-like molecule α (SEQ ID NO:7).

The invention includes a composition comprising an isolated polypeptide comprising a mammalian resistin-like molecule, and a pharmaceutically-acceptable carrier.

In one aspect, the mammalian resistin-like molecule shares at least about 30% sequence identity with at least one amino acid sequence selected from the group consisting of SEQ ID NO:2, SEQ ID NO:4, SEQ ID NO:6, SEQ ID NO:8, and SEQ ID NO:13.

The invention includes a transgenic non-human mammal comprising an isolated nucleic acid encoding a mammalian resistin-like molecule, or a fragment thereof.

In one aspect, the isolated nucleic acid shares at least about 30% sequence identity with a nucleic acid encoding at least one of a mouse resistin-like molecule β (SEQ ID NO:1), a human resistin-like molecule β (SEQ ID NO:3), a mouse resistin-like molecule α (SEQ ID NO:5), and a rat resistin-like molecule α (SEQ ID NO:7).

The invention includes a method of treating a disease mediated by malexpression of a resistin-like molecule alpha in a human, the method comprising administering to a human patient afflicted with a disease mediated by malexpression of a resistin-like molecule a, a resistin-like molecule α expression-inhibiting amount of a composition comprising an isolated nucleic acid complementary to an isolated nucleic acid encoding a resistin-like molecule a, or a fragment thereof, the complementary nucleic acid being in an antisense orientation, the composition further comprising a pharmaceutically-acceptable carrier.

In one aspect, the isolated nucleic acid encoding resistin-like molecule α shares at least about 30% sequence identity with at least one of a nucleic acid encoding a mouse resistin-like molecule β (SEQ ID NO:1), a human resistin-like molecule β (SEQ ID NO:3), a mouse resistin-like molecule α (SEQ ID NO:5), and a rat resistin-like molecule α (SEQ ID NO:7).

In another aspect, the disease is selected from the group consisting of breast cancer, tongue cancer, insulin resistance, diabetes, Syndrome X, and obesity.

The invention also includes a method of treating a disease mediated by malexpression of a resistin-like molecule β in a human, the method comprising administering to a human patient afflicted with a disease mediated by malexpression of a resistin-like molecule β, a resistin-like molecule β expression-inhibiting amount of a composition comprising an isolated nucleic acid complementary to an isolated nucleic acid encoding a resistin-like molecule β, or a fragment thereof, the complementary nucleic acid being in an antisense orientation, the composition further comprising a pharmaceutically-acceptable carrier.

In one aspect, the isolated nucleic acid encoding resistin-like molecule β shares at least about 30% sequence identity with at least one of a nucleic acid encoding a mouse resistin-like molecule β (SEQ ID NO: 1), a human resistin-like molecule β (SEQ ID NO:3), a mouse resistin-like molecule α (SEQ ID NO:5), and a rat resistin-like molecule α (SEQ ID NO:7).

In another aspect, the disease is selected from the group consisting of irritable bowel disease, inflammatory bowel disease, colon cancer, familial adenomatous polyposis, and an intestinal tumor.

The invention includes a method of diagnosing a colon tumor in an animal, the method comprising obtaining a biological sample from a first animal, assessing the level of resistin-like molecule β in the biological sample, and comparing the level of resistin-like molecule β in the biological sample with the level of resistin-like molecule β in a biological sample obtained from a second otherwise identical animal, wherein a higher level of resistin-like moleculeβ in the biological sample from the first animal compared with the level of resistin-like molecule β in the biological sample from the second otherwise identical animal is an indication that first the animal is afflicted with a colon tumor, thereby diagnosing a colon tumor in an animal.

In one aspect, the biological sample is selected from the group consisting of a blood sample, a lung biopsy sample, a fat biopsy sample, a stool sample, and a cerebrospinal fluid sample.

The invention includes a method of diagnosing familial adenomatous polyposis in an animal. The method comprises obtaining a biological sample from a first animal, assessing the level of resistin-like molecule β in the biological sample, and comparing the level of resistin-like molecule β in the biological sample with the level of resistin-like molecule β in a biological sample obtained from a second otherwise identical animal not afflicted with inflammatory bowel disease, wherein a higher level of resistin-like molecule β in the biological sample from the first animal compared with the level of resistin-like molecule β in the biological sample from the second otherwise identical animal is an indication that the first animal is afflicted with familial adenomatous polyposis, thereby diagnosing familial adenomatous polyposis in an animal.

The invention includes a method of diagnosing inflammatory bowel disease in an animal. The method comprises obtaining a biological sample from a first animal, assessing the level of resistin-like molecule β in the biological sample, and comparing the level of resistin-like molecule β in the biological sample with the level of resistin-like molecule β in a biological sample obtained from a second otherwise identical animal not afflicted with inflammatory bowel disease, wherein a higher level of resistin-like molecule β in the biological sample from the first animal compared with the level of resistin-like molecule β in the biological sample from the second otherwise identical animal is an indication that the first animal is afflicted with inflammatory bowel disease, thereby diagnosing inflammatory bowel disease in an animal.

The invention includes a method of diagnosing insulin resistance in an animal. The method comprises obtaining a biological sample from a first animal, assessing the level of resistin-like molecule α in the biological sample, and comparing the level of resistin-like molecule a in the biological sample with the level of resistin-like molecule α in a biological sample obtained from a second otherwise identical animal not afflicted with insulin resistance, wherein a higher level of resistin-like molecule α in the biological sample from the first animal compared with the level of resistin-like molecule α in the biological sample from the second otherwise identical animal is an indication that the first animal is afflicted with insulin resistance, thereby diagnosing insulin resistance in an animal.

The invention also includes a method of diagnosing diabetes in an animal. The method comprises obtaining a biological sample from a first animal, assessing the level of resistin-like molecule α in the biological sample, and comparing the level of resistin-like molecule α in the biological sample with the level of resistin- like molecule α in a biological sample obtained from a second otherwise identical animal not afflicted with diabetes, wherein a higher level of resistin-like molecule α in the biological sample from the first animal compared with the level of resistin-like molecule α in the biological sample from the second otherwise identical animal is an indication that the first animal is afflicted with diabetes, thereby diagnosing diabetes in an animal.

The invention includes a method of assessing the effectiveness of thiazolidinedione (TZD) therapy in an animal. The method comprises assessing a level of a resistin-like molecule in the animal before, during or after administration of a TZD to the animal, wherein a higher or lower level of the resistin-like molecule in the animal during or after administration of the TZD compared with the level of the resistin-like molecule in the animal before administration of the TZD is an indication of the effectiveness of the TZD therapy in the animal, thereby assessing the effectiveness of TZD therapy in the animal.

In one aspect, the thiazolidinedione is selected from the group consisting of a rosiglitazone, a troglitazone, and a pioglitazone.

The invention includes a method of identifying a compound that affects expression of resistin-like molecule in a cell. The method comprises contacting a cell with a test compound and comparing the level of resistin-like molecule expression in the cell with the level of resistin-like molecule expression in an otherwise identical cell not contacted with the test compound, wherein a higher or lower level of resistin-like molecule expression in the cell contacted with the test compound compared with the level of resistin-like molecule expression in the otherwise identical cell not contacted with the test compound is an indication that the test compound affects expression of resistin-like molecule in a cell, thereby identifying a compound that affects expression of resistin-like molecule in a cell. The invention includes a compound identified by this method.

In one aspect, the resistin-like molecule is selected from the group consisting of a resistin-like molecule α and a resistin-like molecule β.

The invention includes a method of identifying a compound that reduces expression of a resistin-like molecule in a cell. The method comprises contacting a cell with a test compound and comparing the level of resistin-like molecule expression in the cell with the level of resistin-like molecule expression in an otherwise identical cell not contacted with the test compound, wherein a lower level of resistin-like molecule expression in the cell contacted with the test compound compared with the level of resistin-like molecule expression in the otherwise identical cell not contacted with the test compound is an indication that the test compound reduces expression of resistin-like molecule in the cell, thereby identifying a compound that reduces expression of resistin-like molecule in a cell. The invention includes a compound identified by this method.

The invention includes a method of identifying a compound that increases expression of resistin-like molecule in a cell. The method comprises contacting a cell with a test compound and comparing the level of resistin-like molecule expression in the cell with the level of resistin-like molecule expression in an otherwise identical cell not contacted with the test compound, wherein a higher level of resistin-like molecule expression in the cell contacted with the test compound compared with the level of resistin-like molecule expression in the otherwise identical cell not contacted with the test compound is an indication that the test compound increases expression of resistin-like molecule in a cell, thereby identifying a compound that increases expression of resistin-like molecule in a cell. The invention includes a compound identified by this method.

The invention includes a method of detecting the presence or absence of fecal matter in a sample. The method comprises assessing the presence or absence of resistin-like molecule β in a sample, wherein the presence of resistin-like molecule β in the sample is an indication that fecal matter is present in the sample, and wherein the absence of resistin-like molecule β in the sample is an indication that fecal matter is absent from the sample, thereby detecting the presence or absence of fecal matter in a sample.

In one aspect, the assessing comprises contacting the sample with an antibody that specifically binds with resistin-like molecule β and detecting binding of the antibody with resistin-like molecule β in the sample.

The invention includes a kit for alleviating a disease mediated by malexpression of a resistin-like molecule in a human. The kit comprises a resistin-like molecule expression-inhibiting amount of a composition comprising an isolated nucleic acid complementary to an isolated nucleic acid encoding a mammalian resistin-like molecule, or a fragment thereof, the complementary nucleic acid being in an antisense orientation, and a pharmaceutically-acceptable carrier, the kit further comprising an applicator, and an instructional material for the use thereof.

In one aspect, the disease is selected from the group consisting of irritable bowel disease, inflammatory bowel disease, familial adenomatous polyposis, an intestinal tumor, diabetes, insulin resistance, obesity, breast cancer, tongue cancer, and colon cancer.

The invention includes a kit for alleviating a disease mediated by malexpression of a resistin-like molecule in a human. The kit comprises a resistin-like molecule expression-inhibiting amount of a composition comprising an antibody that specifically binds with a mammalian resistin-like molecule polypeptide, or a fragment thereof, and a pharmaceutically-acceptable carrier, the kit further comprising an applicator, and an instructional material for the use thereof.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiment(s) which are presently preferred. It should be understood, however, that the invention is not limited to the precise arrangements and instrumentalities shown. In the drawings:

FIG. 1A is a diagram depicting the nucleic acid sequence of mouse RELMβ (SEQ ID NO:1).

FIG. 1B is a diagram depicting a comparison of the various domains of mouse RELMβ and mouse Resistin proteins. Percent identity is indicated for each domain.

FIG. 1C is an image depicting a Northern blot depicting the tissue-specific expression of mouse RELMβ. A multiple mouse tissue Northern blot (Clontech Labs.) comprising RNA isolated from heart (H), brain (B), spleen (SP), lung (LU), liver (LI), skeletal muscle (SM), kidney (K), testis (TE), colon (C), stomach (ST), white adipose tissue (WA), and brown adipose tissue (BA) was probed using mouse RELMβ cDNA. Along the bottom edge of the Northern blot is an image depicting an ethidium bromide stain of the same gel used for Northern blotting demonstrating the loading of RNA in each gel well. The location of 28S and 18S RNA markers is indicated on the Northern blot.

FIG. 1D is a diagram comparing the amino acid sequence of human RELMβ (SEQ ID NO:4), and mouse RELMβ (SEQ ID NO:2). The consensus merged cDNA sequence is shown in the middle line between the mouse and human sequences.

FIG. 1E is an image depicting a Northern blot depicting the tissue-specific expression of human RELMβ. A multiple mouse tissue Northern blot comprising RNA isolated from brain (B), heart (H), skeletal muscle (SM), colon (C), thymus (TH), spleen (SP), kidney (K), liver (LI), small intestine (SI), placenta (P), lung (LU), and white blood cells (WBC) was probed using human RELMβ cDNA. The position of various size markers (in kb) is indicated along the left edge of the image of the blot.

FIG. 2A is an image depicting a Northern blot depicting the expression of mouse RELMβ in various intestinal tissues. A multiple mouse tissue Northern blot comprising RNA isolated from duodenum (D), proximal jejunum (PJ), distal jejunum (DJ), ileum (I), proximal colon (PC), distal colon (DC), and caecum (C) was probed using mouse RELMβ cDNA. The position of RELMβ mRNA is indicated along the left edge of the image of the blot. The detection of actin, as an internal standard demonstrating proper loading of each gel lane, is demonstrated along the bottom portion of the image. Embryonic day (ED).

FIG. 2B is an image depicting a Northern blot depicting the expression of mouse RELMβ in mouse embryo tissues at various time points in embryonic development. A Northern blot comprising RNA isolated from 7 day, 11, 15, or 17 day mouse embryo was probed using mouse RELMβ eDNA. The position of RELMβ mRNA is indicated along the left edge of the image of the blot.

FIG. 2C is an image depicting in situ hybridization using mouse antisense RELMβ probe demonstrating expression of RELMβ in mouse colon primarily localized in the bases of colonic crypts.

FIG. 2D is an image depicting in situ hybridization using a control mouse sense RELMβ probe demonstrating the specificity of the antisense RELMβ probe used to detect expression of RELMβ in mouse colon as depicted in FIG. 2C, supra.

FIG. 2E is an image depicting a Northern blot analysis demonstrating the expression of RELMβ in tumor (T) and adjacent normal-appearing (N) small intestine tissues obtained from duodenum (D), proximal jejunum (PJ), and distal jejunum (DJ) of min mice. The expression of RELMβ in tissues obtained from the ileum (I) and proximal colon (PC) of mice is also depicted.

FIG. 3A is a diagram comparing the amino acid sequence of mouse RELMα (SEQ ID NO:6), and rat RELMα (SEQ ID NO:8). The consensus merged cDNA sequence is shown in the middle line between the mouse and rat sequences. Identical amino acid residues are indicated, conservative amino acid substitutions are indicated by “+”, and conserved cysteine residues are shaded.

FIG. 3B is a schematic diagram depicting a comparison of the various domains of mouse RELMα, rat RELMα, and mouse Resistin proteins. Percent identity is indicated for each domain.

FIG. 3C is an image of a Northern blot depicting RELMα expression in various mouse tissues, i.e., heart (H), brain (B), pituitary (P), spleen (SP), lung (LU), liver (LI), kidney (K), testis (TE), colon (C), small intestine (SI), tongue (TO), white adipose (WA), and mammary tissue (M).

FIG. 3D is an image depicting a Northern blot demonstrating RELMα expression is detectable in white adipose tissue (WAT) but not in 3T3-L1 adipocytes (L1 Ad). Expression of resistin in both 3T3-L1 adipocytes and white adipose tissue is depicted in the bottom portion of the image.

FIG. 4A is an image depicting a Northern blot demonstrating that RELMs are secreted proteins. Resistin, RELMβ, and RELMα were fused to Flag epitope at the C-terminus and the fusion proteins were expressed in transfected 293T cells. Media from the cell culture was analyzed by immunoblotting using mouse monoclonal anti-flag. The proteins present in media obtained from cells transfected with the control vector were analyzed and is indicated by “−”.

FIG. 4B is a diagram comparing the amino acid sequence of mouse resistin (SEQ ID NO: 10), human resistin (SEQ ID NO: 12), and mouse RELMβ (SEQ ID NO:2), human RELMβ (SEQ ID NO:4), mouse RELMα (SEQ ID NO:6), and rat RELMα (SEQ ID NO:8). The consensus merged cDNA sequence is shown at the top of the figure. The “signature” sequence characteristic of the RELM family members is indicated below the consensus sequence. Shading indicates amino acid identity shared by two or more RELM family members as aligned using the DNASTAR megalign program according to the Jotun Hein method (Bucka-Larsen et al., 1999, Bioinformatics. 15:122-130).

FIG. 5 is an image depicting the amino acid sequence of mouse RELMβ (SEQ ID NO:2).

FIG. 6A is an image depicting the nucleic acid sequence of human RELMβ (SEQ ID NO:3).

FIG. 6B is an image depicting the amino acid sequence of human RELMβ (SEQ ID NO:4).

FIG. 7A is an image depicting the nucleic acid sequence of mouse RELMα (SEQ ID NO:5).

FIG. 7B is an image depicting the amino acid sequence of mouse RELMα (SEQ ID NO:6).

FIG. 7C is an image depicting the amino acid sequence of mouse alternately translated RELMα (SEQ ID NO: 13).

FIG. 8A is an image depicting the nucleic acid sequence of rat RELMα (SEQ ID NO:7).

FIG. 8B is an image depicting the amino acid sequence of rat RELMα (SEQ ID NO:8).

FIG. 9A is an image depicting the nucleic acid sequence of mouse resistin (SEQ ID NO:9).

FIG. 9B is an image depicting the amino acid sequence of mouse resistin (SEQ ID NO: 10).

FIG. 10A is an image depicting the nucleic acid sequence of human resistin (SEQ ID NO:11).

FIG. 10B is an image depicting the amino acid sequence of human resistin (SEQ ID NO:12).

FIG. 11 is an image depicting a Northern blot demonstrating that RELMα mRNA is detectable almost exclusively in stromal vascular (SV, also stromovascular) cells, whereas resistin RNA is detectable almost exclusively in adipocyte cells (Ad). Briefly, 20 μg total RNA were loaded onto each gel lane and hybridized using cDNA probes for resistin and RELMα. Resistin expression was detected almost exclusively in adipocytes (Ad), while RELMα expression was detected almost exclusively in the SV compartment of white adipose tissue. Small amounts of resistin detected in SV and RELMα detected in Ad are likely minor contamination related to the method of enrichment of the two fractions.

FIG. 12 is an image depicting a Northern blot demonstrating the increased expression of RELMα in white adipose tissue due to rosiglitazone. Ob/ob mice, an art-recognized model of diabetes, insulin resistance, were treated with or without rosiglitazone and epididymal white adipose tissue was collected from the mice and was analyzed using Northern blot analysis to assess the level of RELMα expression. That is, 20 μsg of total RNA were loaded onto each gel lane and the blots were hybridized using cDNA probes specific for resistin and RELMα. Resistin expression decreased upon administration of rosiglitazone, whereas expression of RELMα RNA increased in epididymal white adipose tissue both relative to the level of expression of these RNAs in white adipose tissue obtained from ob/ob mice treated with control vehicle.

FIG. 13 is an image depicting a Western blot demonstrating increased RELMβ protein expression in normal (N) mice compared with otherwise identical mice in a germ-free (GF) environment. The first lane shows a control protein with a Flag tag (thereby running slightly higher in molecular weight). The next five lanes demonstrate copious RELMβ protein expression in the stool of normal mice. Mice in a germ free environment have dramatically less RELMβ protein in their stool (last five right-most lanes).

FIG. 14A is an image depicting a Northern blot demonstrating the higher levels of RELMβ RNA expression in the colon of normal (N) mice compared with germ-free mice (GF). Other genes are shown for comparison (i.e., DRA and c-Myc).

FIG. 14B is an image depicting a Western blot demonstrating the higher levels of RELMβ protein in colon samples obtained from mice raised under normal (N) conditions compared with levels of RELMβ protein in mice raised in germ-free (GF) conditions.

FIG. 15 is an image depicting a Western blot demonstrating the level of RELMβ protein in normal human stool. The first lane shown a control (C) protein with a Flag tag (thereby running slightly higher in molecular weight). The next five lanes demonstrate detectable RELMβ protein expression in the stool of normal humans (HS).

FIG. 16 is an image depicting a Northern blot demonstrating the levels of RELMβ RNA in a human intestinal cell line (LS124T cells). Increasing concentrations of rosiglitazone, which is a PPARγ ligand previously shown to improve inflammatory bowel disease, led to dose-dependent increase in RELMβ gene expression.

### DETAILED DESCRIPTION OF THE INVENTION

The invention relates to a novel nucleic acid encoding a mammalian resistin-like molecule beta (RELMβ) and proteins encoded thereby. The invention further relates to a novel nucleic acid encoding a mammalian α RELM (RELMα; previously referred to as RELMγ) and RELMα protein encoded thereby. RELMs are secreted proteins with significant sequence homology with cytokine-like signaling molecules. Moreover, resistin, another novel protein which is highly homologous with RELM family members, shares extensive sequence identity especially at or about the C-terminus where the proteins share a conserved array of cysteine residues.

Resistin, which is a novel protein disclosed in PCT Application which has been assigned International Application No. PCT/US00/11272, now published as International Publication No. WO 00/64920 (international publication date Nov. 2, 2000), has been isolated in both mice and humans. Moreover, International Publication No. WO 00/64920, discloses the nucleic acid sequence of mouse (SEQ ID NO:9) and human (SEQ ID NO:11) resistin, as well as the amino acid sequence of resistin for both mouse (SEQ ID NO:10) and human (SEQ ID NO:12). These sequences are also provided herein.

This invention discloses a new family of hormones related to resistin, a recently discovered adipocyte derived hormone that circulates in the bloodstream and influences glucose metabolism in diabetes and obesity (resistin is disclosed in International Publication No. WO 00/64920, which is incorporated by reference as if set forth in its entirety herein). These new hormones are called resistin-like molecules (RELMs), and are secreted proteins. Two have been characterized in detail are described herein—RELMβ and RELMα. RELMβ is specifically expressed in the gastrointestinal tract and particularly the colon, and is likely to be a critical factor in multiple gastrointestinal diseases.

Resistin plays an important role in glucose metabolism including mediating an effect on glucose tolerance associated with diabetes and obesity in mammals. Further, resistin is secreted from adipocytes and its expression is associated with a high fat diet in mammals. The disclosure of International Publication No. WO 00/64920 is hereby incorporated by reference as if set forth herein in its entirety.

The high degree of sequence identity shared between resistin and RELMα and RELMβ indicates that these molecules are also involved in glucose metabolism, including playing a role in diabetes, Syndrome X, insulin resistance, and obesity. This is further supported by the disclosure provided herein demonstrating that the powerful antidiabetics—TZDs—specifically induce expression of both RELMα and RELMβ and the discovery that RELMα is specifically expressed in white adipose tissue in stromovascular cells therein.

Further, as more fully set forth elsewhere herein, RELMα and/or RELMβ play a role in gastrointestinal diseases, disorders and conditions, including, but not limited to, colon cancer, intestinal tumors, familial adenomatous polyposis, breast cancer, tongue cancer, irritable bowel disease, and inflammatory bowel disease. This is supported by the data disclosed herein demonstrating that RELM β is copiously expressed in the intestines, especially in the colon,

It has also been discovered, as disclosed herein, that expression of RELMβ is markedly increased in colonic tumors in min mice, which is an art-recognized model for human familial adenomatous polyposis. Further, expression of RELMβ is particularly increased in tumors immediately adjacent to normal tissue such that RELMβ expression is greatest in intestinal epithelial cells whose proliferative rate is increased due to normal and/or pathological mechanisms.

Additionally, the data disclosed herein demonstrate that RELMβ is localized in mucous droplets in intestinal goblet cells, which are known to be involved in secretion of their contents into the intestinal lumen. Moreover, it has been discovered that RELMβ is found in copious amounts in mouse stool and is expressed in the colon. Further, the data disclosed herein demonstrate the expression of RELMβ is greatly decreased in mice grown in germ-free conditions, which do not have bacteria in their colon, compared with RELMβ expression in otherwise identical mice grown under normal conditions.

In addition, it has been discovered that RELMβ expression is increased by the powerful antidiabetic thiazolidinedione (TZD) Rosiglitazone. TZDs are known to improve inflammatory bowel disease, further suggesting that RELMβ plays a role in diseases, disorders or conditions associated with bacteria being present in the gut, including, but not limited to, inflammatory bowel disease.

These data demonstrate that RELMβ is associated with intestinal cell proliferation and abnormal cell growth and is further associated with bacteria being present in the intestine. This is important because, to date, there are no reliable, efficient non-invasive diagnostic and therapeutic methods to detect and treat abnormal intestinal cell growth associated with diseases associated with intestinal cell proliferation including, but not limited to, colon cancer, familial adenomatous polyposis, and irritable bowel disease.

It is also important that RELM β expression is correlated with the presence of bacteria in the colon since bacteria are known to play a causative role in inflammatory bowel disease. These data indicate that RELM β expression is correlated with the presence of bacteria in the gut and with diseases, disorders or conditions associated with such bacteria, including, but not limited to, inflammatory bowel disease. Further, RELM β plays a role in and/or is associated with colon cancer, familial adenomatous polyposis, irritable bowel disease, intestinal tumors, breast cancer, and tongue cancer.

There is great interest in identifying a gene which is diagnostic for and/or associated with a disease, disorder or condition associated with intestinal cell proliferation and or intestinal response to bacteria, and RELMβ is the first such gene to be identified as demonstrated by the data disclosed herein.

The data disclosed herein suggests that expression of RELMβ is associated with intestinal cell proliferation and may be up-regulated during colonic tumor growth and/or polyposis. The instant invention provides in vitro and in vivo models for the study of the function and role(s) of RELMs in cell processes, colon cancer, familial adenomatous polyposis, irritable bowel disease, inflammatory bowel disease, intestinal tumors, breast cancer, tongue cancer, diabetes, insulin resistance, and obesity, as well as for the development of therapeutics useful for treating and diagnosing diseases, disorders or conditions associated with altered expression of RELMs.

In addition, the invention relates to another RELM family member, RELMα (previously referred to as RELMγ), which, like resistin, is most abundant in white adipose tissue. Unlike resistin, RELMα is also specifically expressed, albeit at lower levels, in tongue, heart, mammary, and lung tissues.

It has also been discovered that expression of RELMα, like RELMβ, is induced by administration of TZDs, i.e., rosiglitazone. These data further suggest that RELMα is associated with, and likely plays a role in, diseases, disorders or conditions of the intestines, as well as those involving glucose metabolism.

Given the role of resistin in diabetes, insulin resistance, and Syndrome X, and due to the high degree of sequence identity among the RELM family members, including resistin, RELMβ and RELMα are important proteins likely involved in cellular signaling and associated with diseases, disorders or conditions such as colon cancer, familial adenomatous polyposis, irritable bowel disease, inflammatory bowel disease, intestinal tumors, breast cancer, tongue cancer, diabetes, insulin resistance, and obesity.

Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, the term “adjacent” is used to refer to nucleotide sequences which are directly attached to one another, having no intervening nucleotides. By way of example, the pentanucleotide 5′-AAAAA-3′ is adjacent the trinucleotide 5′-TTT-3′ when the two are connected thus: 5′-AAAAATTT-3′ or 5′-TTTAAAAA-3′, but not when the two are connected thus: 5′-AAAAACTTT-3′.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, to “alleviate” colon cancer, familial adenomatous polyposis, irritable bowel disease, inflammatory bowel disease, intestinal tumors, breast cancer, tongue cancer, diabetes, insulin resistance, and obesity, means reducing the severity of one or more symptoms of colon cancer, familial adenomatous polyposis, irritable bowel disease, inflammatory bowel disease, intestinal tumors, breast cancer, tongue cancer, diabetes, insulin resistance, and obesity. This can include, but is not limited to, reducing the level of RELM (β or α, or both) expressed in a cell or tissue, reducing the level of cell proliferation, reducing or increasing the level of RELM in the bloodstream or in the central nervous system including the cerebrospinal fluid, and the like, in a patient, compared with the level of RELM in the patient prior to or in the absence of the method of treatment.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator” as the term is used herein, is meant any device including, but not limited to, a hypodermic syringe, a pipette, and the like, for administering the RELM nucleic acid, protein, and/or composition of the invention to a mammal.

“Biological sample,” as that term is used herein, means a sample obtained from an animal that can be used to assess the level of expression of a RELM, the level of RELM protein present, or both. Such a sample includes, but is not limited to, a blood sample, an intestinal tissue sample, a tongue tissue sample, a heart tissue sample, a mammary gland tissue sample, a lung tissue sample, and a white adipose tissue sample.

By “candidate anti-RELM drug,” as the term is used herein, is meant a compound that when contacted with a cell, reduces the level of expression of a nucleic acid encoding a RELM protein, e.g., RELMβ and RELMα, in the cell compared with the level of RELM expression in that cell prior to contacting the cell with the compound or which reduces the level of expression in the cell compared with the level of RELM expression in an otherwise identical cell which is not contacted with the compound.

By “complementary to a portion or all of the nucleic acid encoding RELM” is meant a sequence of nucleic acid which does not encode a RELM protein. Rather, the sequence which is being expressed in the cells is identical to the non-coding strand of the nucleic acid encoding a RELM protein and thus, does not encode RELM protein.

The terms “complementary” and “antisense” as used herein, are not entirely synonymous. “Antisense” refers particularly to the nucleic acid sequence of the non-coding strand of a double stranded DNA molecule encoding a protein, or to a sequence which is substantially homologous to the non-coding strand. “Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g. amino acid residues in a protein export signal sequence). “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., retroviruses, adenoviruses, and adeno-associated viruses) that incorporate the recombinant polynucleotide.

“Familial adenomatous polyposis,” as the term is used herein, refers to a genetic disease associated with mutations in the adenomatous polyposis coli (APC) gene (Bodmer et al., 1999, Cytogenet. Cell Genet. 86:99-104).

By the term “fecal matter,” as used herein, is meant any excrement or by-product of digestion or substance that passes through the intestinal tract of an animal. Preferably, the matter passes through the colon and is excreted from the animal.

“Insulin resistance,” as the term is used herein, includes reduced or absent responsiveness to the actions of insulin in any or all cells and organs.

By the term “irritable bowel disease,” as the term is used herein, is meant a condition that includes Crohn's disease and ulcerative colitis (Ghosh et al., 2000, B.M.J. 320:1119-1123). These are disabling diseases, which in the case of ulcerative colitis, are associated with colon cancer. The disease mechanism is unknown, diagnosis is difficult, and treatments are empiric.

As used herein, “irritable bowel disease” is a non-life threatening but extremely conunon and disabling condition, which is difficult to diagnose with certainty and whose etiology is completely unknown (Hanuner & Talley, 1999, Am. J. Med. 107:5S-11S).

A first region of an oligonucleotide “flanks” a second region of the oligonucleotide if the two regions are adjacent one another or if the two regions are separated by no more than about 1000 nucleotide residues, and preferably no more than about 100 nucleotide residues.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As applied to a protein, a “fragment” of RELM is about 20 amino acids in length. More preferably, the fragment of a RELM is about 30 amino acids, even more preferably, at least about 40, yet more preferably, at least about 60, even more preferably, at least about 80, yet more preferably, at least about 100, even more preferably, about 100, and more preferably, at least about 110 amino acids in length.

A “genomic DNA” is a DNA strand which has a nucleotide sequence homologous with a gene. By way of example, both a fragment of a chromosome and a cDNA derived by reverse transcription of a mammalian mRNA are genomic DNAs.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

In addition, when the terms “homology” or “identity” are used herein to refer to the nucleic acids and proteins, it should be construed to be applied to homology or identity at both the nucleic acid and the amino acid sequence levels.

A first oligonucleotide anneals with a second oligonucleotide with “high stringency” or “under high stringency conditions” if the two oligonucleotides anneal under conditions whereby only oligonucleotides which are at least about 60%, more preferably at least about 65%, even more preferably at least about 70%, yet more preferably at least about 80%, and preferably at least about 90% or, more preferably, at least about 95% complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g., Sambrook et al., 1989, Molecular Cloning: A Laboratory Manual, Cold Spring Harbor Laboratory, New York).

The detennination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example, at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs), which have a nucleotide sequence which differs from that of the mouse proteins described herein are within the scope of the invention. Nucleic acid molecules corresponding to natural allelic variants and homologs of a cDNA of the invention can be isolated based on their identity to mouse nucleic acid molecules using the mouse cDNAs, or a portion thereof, as a hybridization probe according to standard hybridization techniques under stringent hybridization conditions. For example, a homolog of a mouse RELM protein of the invention can be isolated based on its hybridization with a nucleic acid molecule encoding all or part of mouse RELM under high stringency conditions.

As used herein, an “instructional material” includes a publication, a recording, a diagram, or any other medium of expression which can be used to communicate the usefulness of the nucleic acid, peptide, and/or composition of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

By the term “malexpression of a resistin-like molecule,” as used herein, is meant that the level of expression of a resistin-like molecule (e.g., mouse RELMβ, human RELMβ, mouse RELMα, and rat RELMα) in a cell is detectably higher or lower than the level of expression of RELM in an otherwise identical cell where the otherwise identical cell is obtained from normal tissue that does not exhibit any detectable disease, disorder or condition associated with or mediated by expression of RELM, such as, but not limited to, colon cancer, familial adenomatous polyposis, irritable bowel disease, inflammatory bowel disease, intestinal tumors, breast cancer, tongue cancer, diabetes, insulin resistance, and obesity.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

Preferably, when the nucleic acid encoding the desired protein further comprises a promoter/regulatory sequence, the promoter/regulatory is positioned at the 5′ end of the desired protein coding sequence such that it drives expression of the desired protein in a cell. Together, the nucleic acid encoding the desired protein and its promoter/regulatory sequence comprise a “transgene.”

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “polyadenylation sequence” is a polynucleotide sequence which directs the addition of a poly A tail onto a transcribed messenger RNA sequence.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”A “portion” of a polynucleotide means at least at least about twenty sequential nucleotide residues of the polynucleotide. It is understood that a portion of a polynucleotide may include every nucleotide residue of the polynucleotide.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding finction (e.g., promoter, origin of replication, ribosome-binding site, etc.) as well.

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574).

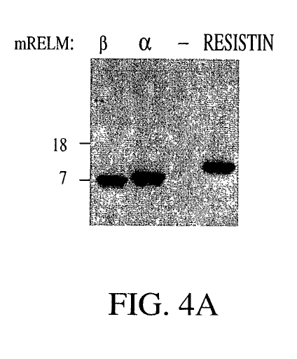

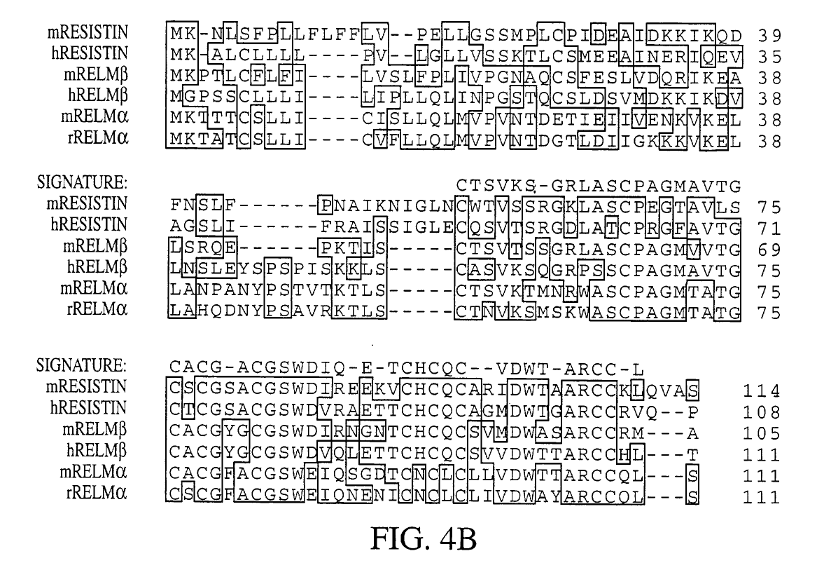

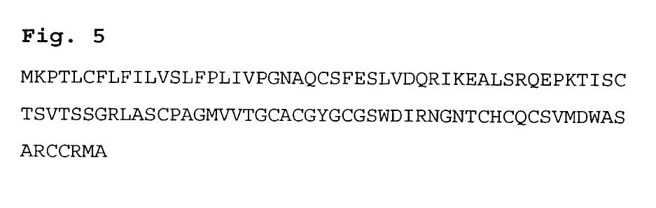

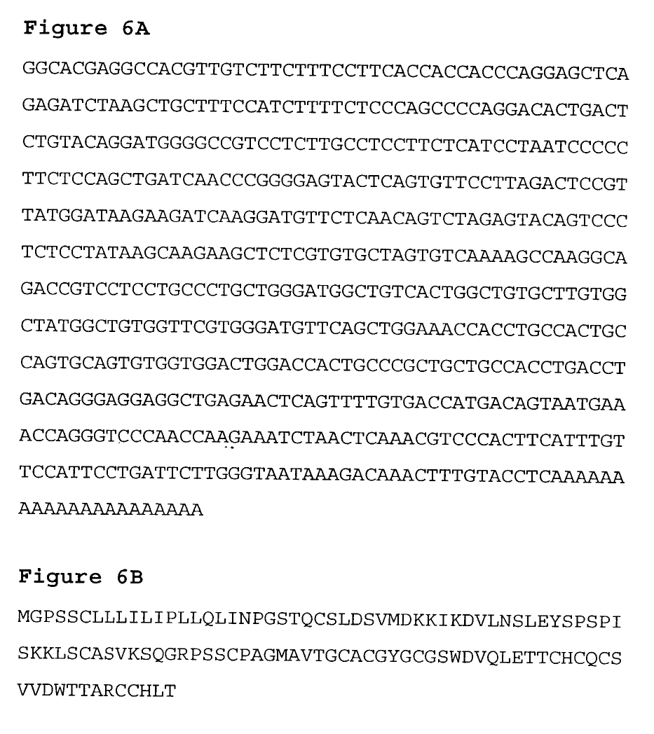

As used herein, the term “RELM” means any resistin-like molecule having significant sequence identity with resistin. Further, for a nucleic acid, the nucleic acid encodes a polypeptide comprising a “signature” sequence as depicted in FIG. 4B. Preferably, the putative RELM encoded by the nucleic acid comprises an invariant sequence of cysteine residues: C-X₁₁-C-X₈-C-X-C-X₃-C-X₁₀-C-X-C-X-C-X₉-CC-X₃₋₆-END. Further, the polypeptide encoded by the nucleic acid comprises a conserved amino acid sequence of “CGSW” and a conserved amino acid sequence of “ARCC.” In the case of a RELM polypeptide or protein, the polypeptide comprises a “signature” sequence as depicted in FIG. 4B. Moreover, the putative RELM comprises an invariant sequence of cysteine residues: C-X₁₁-C-X₈-C-X-C-X₃-C-X₁₀-C-X-C-X-C-X₉-CC-X₃₋₆-END. Further, the RELM polypeptide of the present invention preferably comprises a conserved amino acid sequence of “CGSW” and a conserved amino acid sequence of “ARCC.” Unless otherwise indicated, “RELM” encompasses all known RELMs and RELMs to be discovered, including but not limited to, mouse and human RELMβ and mouse and rat RELMα.

“RELM-inhibiting amount,” as used herein, means any amount of a substance or molecule that detectably decreases the level of RELM expression, amount, and/or activity compared with the level of RELM expression, amount, and/or activity in the absence of the substance or molecule. Thus, any amount that mediates a detectable decrease in: the amount of RELM present, the level of RELM mRNA expression, and/or the ability of RELM to form necessary ligand/receptor interactions, is encompassed in the present invention. The assays by which these conditions are examined are well-known in the art and several are exemplified herein.

By the term “RELMα-like activity,” as used herein, refers to the ability of a molecule or compound to be expressed in white adipose tissue, to be detected in stromovascular cells, to be secreted from a cell, to be induced by TZDs, and the like. “RELMα activity” includes the effects of RELMα, either that circulating in the bloodstream or cerebrospinal fluid or that produced locally in the mammary gland, tongue, lung, heart, or white adipose tissue. RELMα-like activity mediates, is associated with, or both, inter alia, breast cancer, tongue cancer, insulin tolerance, diabetes, Syndrome X, obesity, and the like.

By the term “RELMβ-like activity,” as used herein, refers to the ability of a molecule or compound to increase intestinal cell proliferation, to be secreted from a cell, to be expressed in mammalian colon, to be expressed at higher level in tumor tissue compared to otherwise identical adjacent non-tumor tissue, to be expressed in tumor tissue of an art-recognized model of familial adenomatous polyposis, to be localized in mucous droplets in intestinal goblet cells, to be detectable in stool, to be decreased in level in stool and in expression in colon in a mammal maintained in germ-free conditions, to be induced by TZDs, and the like. “RELMβ activity” includes the effects of RELMβ, either that those mediated by the molecule circulating in the bloodstream or in the cerebrospinal fluid or that produced locally in the intestine, particularly in the colon. RELMβ activity mediates, is associated with, or both, inter alia, colon cancer, intestinal tumors, familial adenomatous polyposis, irritable bowel disease, inflammatory bowel disease, and the like.

A “restriction site” is a portion of a double-stranded nucleic acid which is recognized by a restriction endonuclease.

A portion of a double-stranded nucleic acid is “recognized” by a restriction endonuclease if the endonuclease is capable of cleaving both strands of the nucleic acid at the portion when the nucleic acid and the endonuclease are contacted.

By the term “specifically binds,” as used herein, is meant a compound, e.g., a protein, a nucleic acid, an antibody, and the like, which recognizes and binds a specific molecule, but does not substantially recognize or bind other molecules in a sample.

A first oligonucleotide anneals with a second oligonucleotide “with high stringency” if the two oligonucleotides anneal under conditions whereby only oligonucleotides which are at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g., Sambrook et al., 1989, Molecular Cloning: A Laboratory Manual, Cold Spring Harbor Laboratory, New York).

As used herein, the term “transgene” means an exogenous nucleic acid sequence which exogenous nucleic acid is encoded by a transgenic cell or mammal.

A “recombinant cell” is a cell that comprises a transgene. Such a cell may be a eukaryotic cell or a prokaryotic cell. Also, the transgenic cell encompasses, but is not limited to, an embryonic stem cell comprising the transgene, a cell obtained from a chimeric mammal derived from a transgenic ES cell where the cell comprises the transgene, a cell obtained from a transgenic mammal, or fetal or placental tissue thereof, and a prokaryotic cell comprising the transgene.

By the term “exogenous nucleic acid” is meant that the nucleic acid has been introduced into a cell or an animal using technology which has been developed for the purpose of facilitating the introduction of a nucleic acid into a cell or an animal.

By “tag” polypeptide is meant any protein which, when linked by a peptide bond to a protein of interest, may be used to localize the protein, to purify it from a cell extract, to immobilize it for use in binding assays, or to otherwise study its biological properties and/or function.

As used herein, the term “transgenic mammal” means a mammal, the germ cells of which comprise an exogenous nucleic acid.

As used herein, to “treat” means reducing the frequency with which symptoms of the colon cancer, familial adenomatous polyposis, irritable bowel disease, inflammatory bowel disease, intestinal tumors, breast cancer, tongue cancer, diabetes, insulin resistance, and obesity, are experienced by a patient.

By the term “vector” as used herein, is meant any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector which is suitable as a delivery vehicle for delivery of the RELM protein or nucleic acid encoding a mammalian RELM, to the patient, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; Intemational Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

A “knock-out targeting vector,” as the term is used herein, means a vector comprising two nucleic acid sequences each of which is complementary to a nucleic acid regions flanking a target sequence of interest which is to be deleted and/or replaced by another nucleic acid sequence. The two nucleic acid sequences therefore flank the target sequence which is to be removed by the process of homologous recombination

### DESCRIPTION

I. Isolated nucleic acids

A. Sense nucleic acids

The present invention includes an isolated nucleic acid encoding a mammalian resistin-like molecule, or a fragment thereof, wherein the nucleic acid shares at least about 30% identity with at least one nucleic acid having the sequence of (SEQ ID NO: 1), (SEQ ID NO:3), (SEQ ID NO:5), and (SEQ ID NO:7). Preferably, the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to at least one of SEQ ID NO: 1, SEQ ID NO:3, SEQ ID NO:5, and SEQ ID NO:7 disclosed herein. Even more preferably, the nucleic acid is at least one of SEQ ID NO: 1, SEQ ID NO:3, SEQ ID NO:5, and SEQ ID NO:7.

The present invention includes an isolated nucleic acid encoding mouse resistin-like molecule beta (mRELMβ), or a fragment thereof, wherein the nucleic acid shares at least about 30% homology with mRELMβ (SEQ ID NO: 1). Preferably, the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to the mRELMβ disclosed herein (SEQ ID NO: 1). Even more preferably, the nucleic acid is SEQ ID NO: 1.

The present invention includes an isolated nucleic acid encoding human resistin-like molecule beta (hRELMβ), or a fragment thereof, wherein the nucleic acid shares at least about 30% homology with hRELMβ (SEQ ID NO:3). Preferably, the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to the hRELMβ disclosed herein (SEQ ID NO:3). Even more preferably, the nucleic acid is SEQ ID NO:3.

The present invention includes an isolated nucleic acid encoding mouse resistin-like molecule alpha (mRELMα), or a fragment thereof, wherein the nucleic acid shares at least about 30% homology with mRELMα (SEQ ID NO:5). Preferably, the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to the mRELMα disclosed herein (SEQ ID NO:5). Even more preferably, the nucleic acid is SEQ ID NO:5.

The present invention includes an isolated nucleic acid encoding rat resistin-like molecule alpha (rRELMα), or a fragment thereof, wherein the nucleic acid shares at least about 30% homology with rRELMα (SEQ ID NO:7). Preferably, the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to the rRELMα disclosed herein (SEQ ID NO:7). Even more preferably, the nucleic acid is SEQ ID NO:7.

In another aspect, the present invention includes an isolated nucleic acid encoding a mammalian resistin-like molecule, or a fragment thereof, wherein the protein encoded by the nucleic acid shares at least about 30% homology with the amino acid sequence of at least one of SEQ ID NO:2, SEQ ID NO:4, SEQ ID NO:6, SEQ ID NO:13, and SEQ ID NO:8. Preferably, the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to at least one of SEQ ID NO:2, SEQ ID NO:4, SEQ ID NO:6, SEQ ID NO:13, and SEQ ID NO:8. Even more preferably, the resistin-like molecule protein encoded by the nucleic acid is at least one of SEQ ID NO:2, SEQ ID NO:4, SEQ ID NO:6, SEQ ID NO: 13, and SEQ ID NO:8.

In another aspect, the present invention includes an isolated nucleic acid encoding mouse resistin-like molecule beta (mRELMβ), or a fragment thereof, wherein the protein encoded by the nucleic acid shares at least about 30% homology with the amino acid sequence of SEQ ID NO:2. Preferably, the protein encoded by the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95 % homologous, and most preferably, about 99% homologous to the mRELMβ disclosed herein (SEQ ID NO:2). Even more preferably, the mRELMβ protein encoded by the nucleic acid is SEQ ID NO:2.

In another aspect, the present invention includes an isolated nucleic acid encoding human resistin-like molecule beta (hRELMβ), or a fragment thereof, wherein the protein encoded by the nucleic acid shares at least about 30% homology with the amino acid sequence of SEQ ID NO:4. Preferably, the protein encoded by the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to the hRELMβ disclosed herein (SEQ ID NO:4). Even more preferably, the hRELMβ protein encoded by the nucleic acid is SEQ ID NO:4.

In another aspect, the present invention includes an isolated nucleic acid encoding mouse resistin-like molecule alpha (mRELMα), or a fragment thereof, wherein the protein encoded by the nucleic acid shares at least about 30% homology with the amino acid sequence of SEQ ID NO:6. Preferably, the protein encoded by the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to the mRELMα disclosed herein (SEQ ID NO:6). Even more preferably, the mRELMα protein encoded by the nucleic acid is SEQ ID NO:6.

One skilled in the art would understand, based upon the disclosure provided herein, that a nucleic acid encoding a mouse RELMα can be alternatively translated to produce a alternate mouse RELMα protein comprising an additional 28 amino acid residues at the N-terminus. Therefore, in another aspect, the present invention includes an isolated nucleic acid encoding mouse resistin-like molecule alpha (mRELMα), or a fragment thereof, wherein the protein encoded by the nucleic acid shares at least about 30% homology with the amino acid sequence of SEQ ID NO: 13. Preferably, the protein encoded by the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to the mRELMα disclosed herein (SEQ ID NO:13). Even more preferably, the mRELMα protein encoded by the nucleic acid is SEQ ID NO:13.

In another aspect, the present invention includes an isolated nucleic acid encoding rat resistin-like molecule alpha (rRELMα), or a fragment thereof, wherein the protein encoded by the nucleic acid shares at least about 30% homology with the amino acid sequence of SEQ ID NO:8. Preferably, the protein encoded by the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to the rRELMα disclosed herein (SEQ ID NO:8). Even more preferably, the rRELMα protein encoded by the nucleic acid is SEQ ID NO:8.

One skilled in the art would appreciate, based upon the disclosure provided herein, that a human RELMα homolog likely exists and can be readily identified and isolated using the methods described herein using the sequence data disclosed herein regarding the highly-conserved rat and mouse homologs. Thus, the present invention encompasses additional RELMs that can be readily identified based upon the disclosure provided herein, including, but not limited to, human RELMα.

The isolated nucleic acid of the invention should be construed to include an RNA or a DNA sequence encoding a RELM protein of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The present invention should not be construed as being limited solely to the nucleic and amino acid sequences disclosed herein. Once armed with the present invention, it is readily apparent to one skilled in the art that other nucleic acids encoding RELM proteins can such as those present in other species of mammals (e.g., ape, gibbon, bovine, ovine, equine, porcine, canine, feline, and the like) be obtained by following the procedures described herein in the experimental details section for the isolation of the mouse, rat, and human RELM nucleic acids encoding RELM polypeptides as disclosed herein (e.g., screening of genomic or cDNA libraries), and procedures that are well-known in the art (e.g., reverse transcription PCR using mRNA samples) or to be developed.

Further, any number of procedures may be used for the generation of mutant, derivative or variant forms of RELM using recombinant DNA methodology well known in the art such as, for example, that described in Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York).

Procedures for the introduction of amino acid changes in a protein or polypeptide by altering the DNA sequence encoding the polypeptide are well known in the art and are also described in Sambrook et al. (1989, supra); Ausubel et al. (1997, supra).

The invention includes a nucleic acid encoding a mammalian RELM wherein the nucleic acid encoding a tag polypeptide is covalently linked thereto. That is, the invention encompasses a chimeric nucleic acid wherein the nucleic acid sequences encoding a tag polypeptide is covalently linked to the nucleic acid encoding at least one of mouse RELMβ, human RELMβ, mouse RELMα, and human RELMα. Such tag polypeptides are well known in the art and include, for instance, green fluorescent protein (GFP), myc, myc-pyruvate kinase (myc-PK), His₆, maltose biding protein (MBP), an influenza virus hemagglutinin tag polypeptide, a flag tag polypeptide (FLAG), and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

The nucleic acid comprising a nucleic acid encoding a tag polypeptide can be used to localize RELM within a cell, a tissue, and/or a whole organism (e.g., a mammalian embryo), detect RELM secreted from a cell, and to study the role(s) of RELM in a cell. Further, addition of a tag polypeptide facilitates isolation and purification of the “tagged” protein such that the proteins of the invention can be produced and purified readily.

B. Antisense nucleic acids

In certain situations, it may be desirable to inhibit expression of RELM and the invention therefore includes compositions useful for inhibition of RELM expression. Thus, the invention features an isolated nucleic acid complementary to a portion or all of a nucleic acid encoding a mammalian resistin-like molecule which nucleic acid is in an antisense orientation with respect to transcription. Preferably, the antisense nucleic acid is complementary with a nucleic acid having at least about 30% homology with at least one of SEQ ID NO: 1, SEQ ID NO:3, SEQ ID NO:5, and SEQ ID NO:7, or a fragment thereof. Preferably, the nucleic acid is about 35% homologous, more preferably, about 35% homologous, more preferably, about 40% homologous, even more preferably, about 45% homologous, more preferably, about 50% homologous, preferably, about 55% homologous, more preferably, about 60% homologous, even more preferably, about 65% homologous, more preferably, about 70% homologous, even more preferably, about 75% homologous, preferably, about 80% homologous, more preferably, about 85% homologous, even more preferably, about 90% homologous, and most preferably, about 95% homologous to a nucleic acid complementary to a portion or all of a nucleic acid encoding a mammalian RELM having the sequence of at least one of SEQ ID NO: 1, SEQ ID NO:3, SEQ ID NO:5, and SEQ ID NO:7, or a fragment thereof, which is in an antisense orientation with respect to transcription. Most preferably, the nucleic acid is complementary to a portion or all of a nucleic acid that is at least one of SEQ ID NO: 1, SEQ ID NO:3, SEQ ID NO:5, and SEQ ID NO:7, or a fragment thereof. Such antisense nucleic acid serves to inhibit the expression, function, or both, of a resistin-like molecule.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, supra; Tullis, 1991, U.S. Pat. No. 5,023,243, incorporated by reference herein in its entirety).

II. Isolated polypeptides

The invention also includes an isolated polypeptide comprising a mammalian resistin-like molecule. Preferably, the isolated polypeptide comprising a mammalian resistin-like molecule is at least about 30% homologous to a polypeptide having the amino acid sequence of at least one of SEQ ID NO:2, SEQ ID NO:4, SEQ ID NO:6, SEQ ID NO:8, and SEQ ID NO:13. Preferably, the isolated polypeptide is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to at least one of SEQ ID NO:2, SEQ ID NO:4, SEQ ID NO:6, SEQ ID NO:8, and SEQ ID NO:13. More preferably, the isolated polypeptide comprising a mammalian RELM is at least one of mouse RELM, rat RELM, and human RELM. Most preferably, the isolated polypeptide comprising a mammalian resistin-like molecule is at least one of SEQ ID NO:2, SEQ ID NO:4, SEQ ID NO:6, SEQ ID NO:8, and SEQ ID NO: 13.

The invention also includes an isolated polypeptide comprising a mammalian resistin-like molecule. Preferably, the isolated polypeptide comprising a mammalian resistin-like molecule is at least about 30% homologous to a polypeptide having the amino acid sequence of SEQ ID NO:2. More preferably, the isolated polypeptide comprising a mammalian RELM is at least about 35%, more preferably, about 40% homologous, even more preferably, about 45% homologous, preferably, about 50% homologous, more preferably, about 55% homologous, even more preferably, about 60% homologous, more preferably, about 65% homologous, even more preferably, about 70% homologous, preferably, about 75% homologous, more preferably, about 80% homologous, even more preferably, about 85% homologous, more preferably, about 90% homologous, even more preferably, about 95% homologous, and more preferably, at least about 99% homologous to mouse RELMβ. More preferably, the isolated polypeptide comprising a mammalian resistin-like molecule is mouse RELMβ. Most preferably, the isolated polypeptide comprising a mammalian resistin-like molecule is SEQ ID NO:2.

The invention also includes an isolated polypeptide comprising a mammalian resistin-like molecule. Preferably, the isolated polypeptide comprising a mammalian resistin-like molecule is at least about 30% homologous to a polypeptide having the amino acid sequence of SEQ ID NO:4. More preferably, the isolated polypeptide comprising a mammalian resistin-like molecule is at least about 35%, more preferably, about 40% homologous, even more preferably, about 45% homologous, preferably, about 50% homologous, more preferably, about 55% homologous, even more preferably, about 60% homologous, more preferably, about 65% homologous, even more preferably, about 70% homologous, preferably, about 75% homologous, more preferably, about 80% homologous, even more preferably, about 85% homologous, more preferably, about 90% homologous, even more preferably, about 95% homologous, and more preferably, at least about 99% homologous to SEQ ID NO:4. More preferably, the isolated polypeptide comprising a mammalian resistin-like molecule is human RELMβ. Most preferably, the isolated polypeptide comprising a mammalian resistin-like molecule is SEQ ID NO:4.

The invention also includes an isolated polypeptide comprising a mammalian resistin-like molecule. Preferably, the isolated polypeptide comprising a mammalian resistin-like molecule is at least about 30% homologous to a polypeptide having the amino acid sequence of SEQ ID NO:6. More preferably, the isolated polypeptide comprising a mammalian resistin-like molecule is at least about 35%, more preferably, about 40% homologous, even more preferably, about 45% homologous, preferably, about 50% homologous, more preferably, about 55% homologous, even more preferably, about 60% homologous, more preferably, about 65% homologous, even more preferably, about 70% homologous, preferably, about 75% homologous, more preferably, about 80% homologous, even more preferably, about 85% homologous, preferably, about 90% homologous, more preferably, about 95% homologous, and even more preferably, at least about 99% homologous to SEQ ID NO:6. More preferably, the isolated polypeptide comprising a mammalian resistin-like molecule is mouse RELMα. Most preferably, the isolated polypeptide comprising a mammalian resistin-like molecule is SEQ ID NO:6.

The invention also includes an isolated polypeptide comprising a mammalian resistin-like molecule. Preferably, the isolated polypeptide comprising a mammalian resistin-like molecule is at least about 30% homologous to a polypeptide having the amino acid sequence of SEQ ID NO:8. More preferably, the isolated polypeptide comprising a mammalian resistin-like molecule is at least about 35%, more preferably, about 40% homologous, even more preferably, about 45% homologous, preferably, about 50% homologous, more preferably, about 55% homologous, even more preferably, about 60% homologous, more preferably, about 65% homologous, even more preferably, about 70% homologous, preferably, about 75% homologous, more preferably, about 80% homologous, even more preferably, about 85% homologous, preferably, about 90% homologous, more preferably, about 95% homologous, and even more preferably, at least about 99% homologous to SEQ ID NO:8. More preferably, the isolated polypeptide comprising a mammalian resistin-like molecule is rat RELMα. Most preferably, the isolated polypeptide comprising a mammalian resistin-like molecule is SEQ ID NO:8.

The invention also includes an isolated polypeptide comprising a mammalian resistin-like molecule. Preferably, the isolated polypeptide comprising a mammalian resistin-like molecule is at least about 30% homologous to a polypeptide having the amino acid sequence of SEQ ID NO:13. More preferably, the isolated polypeptide comprising a mammalian resistin-like molecule is at least about 35%, more preferably, about 40% homologous, even more preferably, about 45% homologous, preferably, about 50% homologous, more preferably, about 55% homologous, even more preferably, about 60% homologous, more preferably, about 65% homologous, even more preferably, about 70% homologous, preferably, about 75% homologous, more preferably, about 80% homologous, even more preferably, about 85% homologous, preferably, about 90% homologous, more preferably, about 95% homologous, and even more preferably, at least about 99% homologous to SEQ ID NO: 13. More preferably, the isolated polypeptide comprising a mammalian resistin-like molecule is mouse RELMα. Most preferably, the isolated polypeptide comprising a mammalian resistin-like molecule is SEQ ID NO: 13.

Preferably, the RELM polypeptide comprises a “signature” sequence as depicted in FIG. 4B. More preferably, the RELM encoded by the nucleic acid of the invention comprises an invariant sequence of cysteine residues: C-X₁₁-C-X₈-C-X-C-X₃-C-X₁₀-C-X-C-X-C-X₉-CC-X₃₋₆-END. Further, the polypeptide encoded by the nucleic acid preferably comprises a conserved amino acid sequence of “CGSW” and a conserved amino acid sequence of “ARCC.” In the case of a RELM polypeptide or protein, the polypeptide comprises a “signature” sequence as depicted in FIG. 4B. More preferably, the RELM polypeptide comprises an invariant sequence of cysteine residues: C-X₁₁-C-X₈-C-X-C-X₃-C-X₁₀-C-X-C-X-C-X₉-CC-X₃₋₆-END. Preferably, the RELM polypeptide of the present invention preferably comprises a conserved amino acid sequence of “CGSW” and a conserved amino acid sequence of “ARCC.”

The present invention also provides for analogs of proteins or peptides which comprise a resistin-like molecule as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:

glycine, alanine;

valine, isoleucine, leucine;

aspartic acid, glutamic acid;

asparagine, glutamine;

senne, threonine;

lysine, arginine;

phenylalanine, tyrosine. Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “mutants,” “derivatives,” and “variants” of the peptides of the invention (or of the DNA encoding the same) which mutants, derivatives and variants are RELM peptides which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the RELM peptide of the present invention.

A biological property of a RELM protein should be construed but not be limited to include, the ability of the peptide to be secreted from a cell, to act locally or via circulating in the bloodstream or in the cerebrospinal fluid, to cause biological changes in a target cell as is typical of hormones, and the like.

The skilled artisan would understand, based upon the disclosure provided herein, that RELMα biological activity encompasses, but is not limited to, the ability of a molecule or compound to be expressed in white adipose tissue, to be detected in stromovascular cells, to be secreted from a cell, to be induced by TZDs, and the like. “RELMα activity” includes the effects of RELMα, either that circulating in the bloodstream or cerebrospinal fluid or that produced locally in the mammary gland, tongue, lung, heart, or white adipose tissue. RELMα biological activity mediates, is associated with, or both, inter alia, breast cancer, tongue cancer, insulin tolerance, diabetes, Syndrome X, obesity, and the like.

One skilled in the art would also appreciated, based on the disclosure provided herein, that RELMβ biological activity includes, but is not limited to, the ability of a molecule or compound to increase intestinal cell proliferation, to be secreted from a cell, to be expressed in mammalian colon, to be expressed at higher level in tumor tissue compared to otherwise identical adjacent non-tumor tissue, to be expressed in tumor tissue of an art-recognized model of familial adenomatous polyposis, to be localized in mucous droplets in intestinal goblet cells, to be detectable in stool, to be decreased in level in stool and in expression in colon in a mammal maintained in germ-free conditions, to be induced by TZDs, and the like. RELMβ biological activity includes the effects of RELMβ, either that those mediated by the molecule circulating in the bloodstream or in the cerebrospinal fluid or that produced locally in the intestine, particularly in the colon. RELMβ activity mediates, is associated with, or both, inter alia, colon cancer, intestinal tumors, familial adenomatous polyposis, irritable bowel disease, inflammatory bowel disease, and the like.

Further, the invention should be construed to include naturally occurring variants or recombinantly derived mutants of RELM sequences, which variants or mutants render the protein encoded thereby either more, less, or just as biologically active as the full-length clones of the invention.

The nucleic acids, and peptides encoded thereby, are useful tools for elucidating the function(s) of resistin-like molecule in a cell. Further, nucleic and amino acids comprising mammalian resistin-like molecule are useful diagnostics which can be used, for example, to identify a compound that affects RELM expression and is a potential intestinal (e.g., colon) anticancer, anti-cell proliferation, anti-inflammatory bowel disease, and anti-irritable bowel disease drug candidate. The nucleic acids, the proteins encoded thereby, or both, can be administered to a mammal to increase or decrease expression of RELM alpha and/or beta in the mammal. This can be beneficial for the mammal in situations where under or over-expression of RELMβ and/or -α in the mammal mediates a disease or condition associated with altered expression of RELM compared with normal expression of RELM in a healthy mammal.

That is, the data disclosed herein demonstrate that malexpression of RELMα is associated with diabetes, insulin resistance, Syndrome X, and obesity. Further, the data disclosed herein demonstrate that malexpression of RELMβ is associated with inter alia, colon cancer, intestinal tumors, familial adenomatous polyposis, irritable bowel disease, inflammatory bowel disease, and the like.

Additionally, the nucleic and amino acids of the invention can be used to produce recombinant cells and transgenic non-human mammals which are useful tools for the study of RELM action, the identification of novel diagnostics and therapeutics for treatment of colon cancer, familial adenomatous polyposis, irritable bowel disease, inflammatory bowel disease, a disease, disorder or condition mediated by or associated with intestinal bacteria, intestinal tumors, breast cancer, tongue cancer, diabetes, insulin resistance, Syndrome X, and obesity, and for elucidating the cellular role(s) of RELM, among other things. Further, the nucleic and amino acids of the invention can be used diagnostically, either by assessing the level of gene expression or protein expression, to assess severity and prognosis of colon tumors, tongue tumors, breast tumors, diabetes, insulin resistance, intestinal tumors, and intestinal polyps. The nucleic acids and proteins of the invention are also useful in the development of assays to assess the efficacy of a treatment for colon tumors, intestinal tumors, tongue tumors, diabetes, insulin resistance, obesity, breast cancer, irritable bowel disease, inflammatory bowel disease, and familial adenomatous polyposis. That is, the nucleic acids and polypeptides of the invention can be used to detect the effect of various therapies on resistin-like molecule expression, thereby ascertaining the effectiveness of the therapies.

III. Vectors

In other related aspects, the invention includes an isolated nucleic acid encoding a mammalian RELM operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (1989, supra), and Ausubel et al. (1997, supra).

Expression of RELM, either alone or fused to a detectable tag polypeptide, in cells which either do not normally express the RELM or which do not express RELM fused with a tag polypeptide, may be accomplished by generating a plasmid, viral, or other type of vector comprising the desired nucleic acid operably linked to a promoter/regulatory sequence which serves to drive expression of the protein, with or without tag, in cells in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, both of which were used in the experiments disclosed herein, as well as the Rous sarcoma virus promoter, and the like. Moreover, inducible and tissue specific expression of the nucleic acid encoding RELM may be accomplished by placing the nucleic acid encoding RELM, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Expressing RELM using a vector allows the isolation of large amounts of recombinantly produced protein. Further, where the lack or decreased level of RELM expression causes a disease, disorder, or condition associated with such expression, the expression of RELM driven by a promoter/regulatory sequence can provide useful therapeutics including, but not limited to, gene therapy whereby RELM is provided. A disease, disorder or condition associated with a decreased level of expression, level of protein, or decreased activity of the protein, for which administration of RELM can be useful can include, but is not limited to, colon cancer, familial adenomatous polyposis, irritable bowel disease, inflammatory bowel disease, intestinal tumors, breast cancer, tongue cancer, diabetes, insulin resistance, and obesity, and the like. Preferably, a disease, disorder or condition associated with a decreased level of RELMα includes, but is not limited to, diabetes, insulin resistance, Syndrome X, breast cancer, tongue cancer, and obesity. Further, a disease, disorder or condition associated with a decreased level of RELMβ includes, but is not limited to, colon cancer, intestinal tumors, familial adenomatous polyposis, irritable bowel disease, inflammatory bowel disease, and the like

Therefore, the invention includes not only methods of inhibiting RELM expression, translation, and/or activity, but it also includes methods relating to increasing RELM expression, protein level, and/or activity since both decreasing and increasing RELM expression and/or activity can be useful in providing effective therapeutics.

Selection of any particular plasmid vector or other DNA vector is not a limiting factor in this invention and a wide plethora vectors is well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook, supra, and Ausubel, supra.

The invention thus includes a vector comprising an isolated nucleic acid encoding a mammalian RELM. The incorporation of a desired nucleic acid into a vector and the choice of vectors is well-known in the art as described in, for example, Sambrook et al., supra, and Ausubel et al., supra.

The invention also includes cells, viruses, proviruses, and the like, containing such vectors. Methods for producing cells comprising vectors and/or exogenous nucleic acids are well-known in the art. See, e.g., Sambrook et al., supra; Ausubel et al., supra.

The nucleic acids encoding RELM may be cloned into various plasmid vectors. However, the present invention should not be construed to be limited to plasmids or to any particular vector. Instead, the present invention should be construed to encompass a wide plethora of vectors which are readily available and/or well-known in the art.

IV. Antisense molecules and ribozymes

Further, the invention includes a recombinant cell comprising an antisense nucleic acid which cell is a useful model for elucidating the role(s) of RELM in cellular processes. That is, the increased expression of RELMβ in min mice indicates that RELM is involved in cell proliferation associated with tumor growth. Accordingly, a transgenic cell comprising an antisense nucleic acid complementary to RELM is a useful tool for the study of the mechanism(s) of action of RELM and its role(s) in the cell and for the identification of therapeutics that ameliorate the effect(s) of RELM expression. Further, methods of decreasing RELM expression and/or activity in a cell can provide useful diagnostics and/or therapeutics for diseases, disorders or conditions mediated by or associated with increased RELM expression, increased level of RELM protein in a cell or secretion therefrom, and/or increased RELM activity. Such diseases, disorders or conditions include, but are not limited to, colon cancer, familial adenomatous polyposis, irritable bowel disease, inflammatory bowel disease, intestinal tumors, and the like, which are mediated by or associated with increased RELMβ expression.

One skilled in the art will appreciate that one way to decrease the levels of RELM mRNA and/or protein in a cell is to inhibit expression of the nucleic acid encoding the protein. Expression of RELM may be inhibited using, for example, antisense molecules, and also by using ribozymes or double-stranded RNA as described in, for example, Wianny and Kemicka-Goetz (2000, Nature Cell Biol. 2:70-75).

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue (1993, U.S. Pat. No. 5,190,931).

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, supra; Tullis, 1991, U.S. Pat. No. 5,023,243, incorporated by reference herein in its entirety).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053, incorporated by reference herein in its entirety). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is that, because they are sequence-specific, only mRNAs with particular sequences are inactivated.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of RELM may be designed by incorporating target sequences into the basic ribozyme structure which are complementary to the mRNA sequence of the RELM encoded by RELM or having at least about 80% homology to at least one of SEQ ID NO: 1 and SEQ ID NO:3. Ribozymes targeting RELM may be synthesized using commercially available reagents (Applied Biosystems, Inc., Foster City, Calif.) or they may be genetically expressed from DNA encoding them.

V. Recombinant cells and transgenic non-human mammals

The invention includes a recombinant cell comprising, inter alia, an isolated nucleic acid encoding RELM, an antisense nucleic acid complementary thereto, a nucleic acid encoding an antibody that specifically binds RELM, and the like. In one aspect, the recombinant cell can be transiently transfected with a plasmid encoding a portion of the nucleic acid encoding RELM. The nucleic acid need not be integrated into the cell genome nor does it need to be expressed in the cell. Moreover, the cell may be a prokaryotic or a eukaryotic cell and the invention should not be construed to be limited to any particular cell line or cell type. Such cells include, but are not limited to, fibroblasts, hepatocytes, skeletal muscle cells, and adipocytes.

In one aspect, the recombinant cell comprising an isolated nucleic acid encoding mammalian RELM is used to produce a transgenic non-human mammal. That is, the exogenous nucleic acid, or transgene as it is also referred to herein, of the invention is introduced into a cell, and the cell is then used to generate the non-human transgenic mammal. The cell into which the transgene is introduced is preferably an embryonic stem (ES) cell. However, the invention should not be construed to be limited solely to ES cells comprising the transgene of the invention nor to cells used to produce transgenic animals. Rather, a transgenic cell of the invention includes, but is not limited to, any cell derived from a transgenic animal comprising a transgene, a cell comprising the transgene derived from a chimeric animal derived from the transgenic ES cell, and any other comprising the transgene which may or may not be used to generate a non-human transgenic mammal.

Further, it is important to note that the purpose of transgene-comprising, i.e., recombinant, cells should not be construed to be limited to the generation of transgenic mammals. Rather, the invention should be construed to include any cell type into which a nucleic acid encoding a mammalian RELM is introduced, including, without limitation, a prokaryotic cell and a eukaryotic cell comprising an isolated nucleic acid encoding mammalian RELM.

When the cell is a eukaryotic cell, the cell may be any eukaryotic cell which, when the transgene of the invention is introduced therein, and the protein encoded by the desired gene is no longer expressed therefrom, a benefit is obtained. Such a benefit may include the fact that there has been provided a system in which lack of expression of the desired gene can be studied in vitro in the laboratory or in a mammal in which the cell resides, a system wherein cells comprising the introduced gene deletion can be used as research, diagnostic and therapeutic tools, and a system wherein animal models are generated which are useful for the development of new diagnostic and therapeutic tools for selected disease states in a mammal including, for example, colon cancer, familial adenomatous polyposis, irritable bowel disease, inflammatory bowel disease, intestinal tumors, breast cancer, tongue cancer, diabetes, insulin resistance, and obesity, and the like.

Alternatively, the invention includes a eukaryotic cell which, when the transgene of the invention is introduced therein, and the protein encoded by the desired gene is expressed therefrom where it was not previously present or expressed in the cell or where it is now expressed at a level or under circumstances different than that before the transgene was introduced, a benefit is obtained. Such a benefit may include the fact that there has been provided a system in the expression of the desired gene can be studied in vitro in the laboratory or in a mammal in which the cell resides, a system wherein cells comprising the introduced gene can be used as research, diagnostic and therapeutic tools, and a system wherein animal models are generated which are useful for the development of new diagnostic and therapeutic tools for selected disease states in a mammal.

Such cell expressing an isolated nucleic acid encoding RELM can be used to provide RFLM to a cell, tissue, or whole animal where a higher level of RELM can be useful to treat or alleviate a disease, disorder or condition associated with low level of RELM expression and/or activity. Such diseases, disorders or conditions can include, but are not limited to, intestinal tumors, familial adenomatous polyposis, colon cancer, irritable bowel disease, inflammatory bowel disease, diabetes, insulin resistance, obesity, tongue cancer, breast cancer, lung cancer, and the like. Therefore, the invention includes a cell expressing RELM to increase or induce RELM expression, translation, and/or activity, where increasing RELM expression, protein level, and/or activity can be useful to treat or alleviate a disease, disorder or condition.

One of ordinary skill would appreciate, based upon the disclosure provided herein, that a “knock-in” or “knock-out” vector of the invention comprises at least two sequences homologous to two portions of the nucleic acid which is to be replaced or deleted, respectively. The two sequences are homologous with sequences that flank the gene; that is, one sequence is homologous with a region at or near the 5′ portion of the coding sequence of the nucleic acid encoding RELM and the other sequence is further downstream from the first. One skilled in the art would appreciate, based upon the disclosure provided herein, that the present invention is not limited to any specific flanking nucleic acid sequences. Instead, the targeting vector may comprise two sequences which remove some or all (i.e., a “knock-out” vector) or which insert (i.e., a “knock-in” vector) a nucleic acid encoding RELM, or a fragment thereof, from or into a mammalian genome, respectively. The crucial feature of the targeting vector is that it comprise sufficient portions of two sequences located towards opposite, i.e., 5′ and 3′, ends of the RELM open reading frame (ORF) in the case of a “knock-out” vector, to allow deletion/insertion by homologous recombination to occur such that all or a portion of the nucleic acid encoding RELM is deleted from or inserted into a location on a mammalian chromosome.

The design of transgenes and knock-in and knock-out targeting vectors is well-known in the art and is described in standard treatises such as Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York), and the like. The upstream and downstream portions flanking or within the RELM coding region to be used in the targeting vector may be easily selected based upon known methods and following the teachings disclosed herein based on the disclosure provided herein including the nucleic and amino acid sequences of both mouse and human RELM. Armed with these sequences, one of ordinary skill in the art would be able to construct the transgenes and knock-out vectors of the invention.

The invention firther includes a knock-out targeting vector comprising a nucleic acid encoding a selectable marker such as, for example, a nucleic acid encoding the neo^{R} gene thereby allowing the selection of transgenic a cell where the nucleic acid encoding RELM, or a portion thereof, has been deleted and replaced with the neomycin resistance gene by the cell's ability to grow in the presence of G418. However, the present invention should not be construed to be limited to neomycin resistance as a selectable marker. Rather, other selectable markers well-known in the art may be used in the knock-out targeting vector to allow selection of recombinant cells where the RELM gene has been deleted and/or inactivated and replaced by the nucleic acid encoding the selectable marker of choice. Methods of selecting and incorporating a selectable marker into a vector are well-known in the art and are describe in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

As noted herein, the invention includes a non-human transgenic mammal comprising an exogenous nucleic acid inserted into a desired site in the genome thereof thereby deleting the coding region of a desired endogenous target gene, i.e., a knock-out transgenic mammal. Further, the invention includes a transgenic non-human mammal wherein an exogenous nucleic acid encoding RELM is inserted into a site the genome, i.e., a “knock-in” transgenic mammal. The knock-in transgene inserted may comprise various nucleic acids encoding, for example, a tag polypeptide, a promoter/regulatory region operably linked to the nucleic acid encoding RELM not normally present in the cell or not typically operably linked to RELM.

The generation of the non-human transgenic mammal of the invention is preferably accomplished using the method which is now described. However, the invention should in no way be construed as being limited solely to the use of this method, in that, other methods can be used to generate the desired knock-out mammal.

In the preferred method of generating a non-human transgenic mammal, ES cells are generated comprising the transgene of the invention and the cells are then used to generate the knock-out animal essentially as described in Nagy and Rossant (1993, In: Gene Targeting, A Practical Approach, pp.146-179, Joyner ed., IRL Press). ES cells behave as normal embryonic cells if they are returned to the embryonic environment by injection into a host blastocyst or aggregate with blastomere stage embryos. When so returned, the cells have the full potential to develop along all lineages of the embryo. Thus, it is possible, to obtain ES cells, introduce a desired DNA therein, and then return the cell to the embryonic environment for development into mature mammalian cells, wherein the desired DNA may be expressed.

Precise protocols for the generation of transgenic mice are disclosed in Nagy and Rossant (1993, In: Gene Targeting, A Practical Approach, Joyner ed. IRL Press, pp. 146-179). and are therefore not repeated herein. Transfection or transduction of ES cells in order to introduce the desired DNA therein is accomplished using standard protocols, such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York). Preferably, the desired DNA contained within the transgene of the invention is electroporated into ES cells, and the cells are propagated as described in Soriano et al. (1991, Cell 64:693-702).

Introduction of an isolated nucleic acid into the fertilized egg of the mammal is accomplished by any number of standard techniques in transgenic technology (Hogan et al., 1986, Manipulating the Mouse Embryo: A Laboratory Manual, Cold Spring Harbor, N.Y.). Most commonly, the nucleic acid is introduced into the embryo by way of microinjection.

Once the nucleic acid is introduced into the egg, the egg is incubated for a short period of time and is then transferred into a pseudopregnant mammal of the same species from which the egg was obtained as described, for example, in Hogan et al. (1986, Manipulating the Mouse Embryo: A Laboratory Manual, Cold Spring Harbor, N.Y.). Typically, many eggs are injected per experiment, and approximately two-thirds of the eggs survive the procedure. About twenty viable eggs are then transferred into pseudopregnant animals, and usually four to ten of the viable eggs so transferred will develop into live pups.

Any mammalian RELM gene may be used in the methods described herein to produce a transgenic mammal or a transgenic cell harboring a transgene comprising a deletion of all or part of that mammalian resistin-like molecule gene. Preferably, a rodent resistin-like molecule gene such as, e.g., mouse RELMβ (SEQ ID NO:I), mouse RELMα (SEQ ID NO:5), and rat RELMα (SEQ ID NO:7), is used, and human RELMβ (SEQ ID NO:3) gene, is also used.

The transgenic mammal of the invention can be any species of mammal. Thus, the invention should be construed to include generation of transgenic mammals encoding the chimeric nucleic acid, which mammals include mice, hamsters, rats, rabbits, pigs, sheep and cattle. The methods described herein for generation of transgenic mice can be analogously applied using any mammalian species. Preferably, the transgenic mammal of the invention is a rodent and even more preferably, the transgenic mammal of the invention is a mouse. By way of example, Lukkarinen et al. (1997, Stroke 28:639-645), teaches that gene constructs which enable the generation of transgenic mice also enable the generation of other transgenic rodents, including rats. Similarly, nullizygous mutations in a genetic locus of an animal of one species can be replicated in an animal of another species having a genetic locus highly homologous to the first species.

To identify the transgenic mammals of the invention, pups are examined for the presence of the isolated nucleic acid using standard technology such as Southern blot hybridization, PCR, and/or RT-PCR. Expression of the nucleic acid in the cells and in the tissues of the mammal is also assessed using ordinary technology described herein. Further, the presence or absence of RELM in the circulating blood of the transgenic animal can be determined, for example, as disclosed herein (e.g., Western blot analysis), or using standard methods for protein detection that are well- known in the art.

Cells obtained from the transgenic mammal of the invention, which are also considered “transgenic cells” as the term is used herein, encompass such as cells as those obtained from the RELM (+/−) and (−/−) transgenic non-human mammal described elsewhere herein, are useful systems for modeling diseases and symptoms of mammals which are believed to be associated with altered levels of RELM expression such as colon cancer, familial adenomatous polyposis, irritable bowel disease, inflammatory bowel disease, intestinal tumors, breast cancer, tongue cancer, diabetes, insulin resistance, and obesity, and any other disease, disorder or condition associated with an altered level of RELM expression. Moreover, as a marker of a pathway(s) associated with tumor proliferation and other intestinal abnormalities such colon

Alternatively, recombinant cells expressing RELM can be administered in ex vivo and in vivo therapies where administering the recombinant cells thereby administers the protein to a cell, a tissue, and/or an animal. Additionally, the recombinant cells are useful for the discovery of RELM receptor and RELM signaling pathways.

The recombinant cell of the invention may be used to study the effects of elevated or decreased RELM levels on cell homeostasis and cell proliferation since RELM has been hypothesized to play a role in colon cancer, familial adenomatous polyposis, irritable bowel disease, inflammatory bowel disease, intestinal tumors, breast cancer, tongue cancer, diabetes, insulin resistance, and obesity, and the like

The recombinant cell of the invention, wherein the cell has been engineered such that it does not express RELM, or expresses reduced or altered RELM lacking biological activity, can also be used in ex vivo and in vivo cell therapies where either an animal's own cells (e.g., adipocytes, intestinal epithelial cells, lung cells, muscle cells, fibroblasts, and the like) or those of a syngeneic matched donor are recombinantly engineered as described elsewhere herein (e.g., by insertion of an antisense nucleic acid or a knock-out vector such that RELM expression and/or protein levels are thereby reduced in the recombinant cell), and the recombinant cell is administered to the recipient animal. In this way, recombinant cells that express RELM at a reduced level can be administered to an animal whose own cells express increased levels of RELM thereby treating or alleviating a disease, disorder or condition associated with or mediated by increased RELM expression as disclosed elsewhere herein.

The transgenic mammal of the invention, rendered susceptible to familial adenomatous polyposis such as, for example, the min mouse which comprises a mutation in the APC gene, can be used to study the pathogenesis of familial adenomatous polyposis and the possible role of RELM therein.

Further, the transgenic mammal and/or cell of the invention may be used to study the subcellular localization of RELM.

Also, the transgenic mammal (both +/− and −/− live born and fetuses) and/or cell of the invention may be used to study to role(s) of RELM in glucose metabolism and to elucidate the target(s) of RELM action as well as any receptor(s) that bind with RELM to mediate its effect(s) in the cell.

VI. Antibodies

The invention also includes an antibody that specifically binds RELM, or a fragment thereof.

One skilled in the art would understand, based upon the disclosure provided herein, that an antibody that specifically binds RELM, binds with a protein of the invention, such as, but not limited to mouse RELMβ, human RELMβ, mouse RELMα, and/or rat RELMα, or an immunogenic portion thereof. In one embodiment, the antibody is directed to: mouse RELMβ comprising the amino acid sequence of SEQ ID NO:2, human RELMβ, comprising the amino acid sequence SEQ ID NO:4, mouse RELMα, comprising the amino acid sequence SEQ ID NO:6, and alternately translated SEQ ID NO: 13, and rat RELMα, comprising the amino acid sequence SEQ ID NO:8.

Polyclonal antibodies are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the RELM portion is rendered immunogenic (e.g., RELM conjugated with keyhole limpet hemocyanin, KLH) and a portion comprising the respective rodent and/or human RELM amino acid residues. The chimeric proteins are produced by cloning the appropriate nucleic acids encoding RELM (e.g., [SEQ ID NO:1] and [SEQ ID NO:3]) into a plasmid vector suitable for this purpose, such as but not limited to, pMAL-2 or pCMX.

However, the invention should not be construed as being limited solely to these antibodies or to these portions of the protein antigens. Rather, the invention should be construed to include other antibodies, as that term is defined elsewhere herein, to mouse and human RELM, or portions thereof Further, the present invention should be construed to encompass antibodies, inter alia, bind to RELM and they are able to bind RELM present on Western blots, in immunohistochemical staining of tissues thereby localizing RELM in the tissues, and in immunofluorescence microscopy of a cell transiently transfected with a nucleic acid encoding at least a portion of RELM.

One skilled in the art would appreciate, based upon the disclosure provided herein, that the antibody can specifically bind with any portion of the protein and the full-length protein can be used to generate antibodies specific therefor. However, the present invention is not limited to using the full-length protein as an immunogen. Rather, the present invention includes using an immunogenic portion of the protein to produce an antibody that specifically binds with mammalian RELM. That is, the invention includes immunizing an animal using an immunogenic portion, or antigenic determinant, of the RELM protein.

The antibodies can be produced by immunizing an animal such as, but not limited to, a rabbit or a mouse, with a protein of the invention, or a portion thereof, or by immunizing an animal using a protein comprising at least a portion of RELM, or a fusion protein including a tag polypeptide portion comprising, for example, a maltose binding protein tag polypeptide portion, covalently linked with a portion comprising the appropriate RELM amino acid residues. One skilled in the art would appreciate, based upon the disclosure provided herein, that smaller fragments of these proteins can also be used to produce antibodies that specifically bind RELM.

One skilled in the art would appreciate, based upon the disclosure provided herein, that various portions of an isolated RELM polypeptide can be used to generate antibodies to either highly conserved regions of RELM or to non-conserved regions of the polypeptide. As disclosed elsewhere herein, RELM comprises various conserved domains including, but not limited to, a putative signal peptide from about amino acid residue 1 to about amino acid residue 20 (in RELMβ), and a secreted portion comprising a highly conserved C-terminal region of about half the amino acid residues of the molecule, wherein the C-terminal portion is further characterized by a signature sequence comprising conserved cysteine residues demonstrating a unique and invariant spacing: C-X₁₁-C-X₈-C-X-C-X₃-C-X₁₀-C-X-C-X-C-X₉-CC-X₃₋₆-END. These cysteine residues are also present in mouse and human resistins (see, e.g., SEQ ID NO: 10 and SEQ ID NO: 12).

Once armed with the sequence of RELM and the detailed analysis localizing the various conserved and non-conserved domains of the protein, the skilled artisan would understand, based upon the disclosure provided herein, how to obtain antibodies specific for the various portions of a mammalian RELM polypeptide using methods well-known in the art or to be developed.

Further, the skilled artisan, based upon the disclosure provided herein, would appreciate that the non-conserved regions of a protein of interest can be more immunogenic than the highly conserved regions which are conserved among various organisms. Further, immunization using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of each RELM molecule can be used to produce antibodies that are specific only for that RELM and do not cross-react non-specifically with other RELMs or with other proteins.

Alternatively, the skilled artisan would also understand, based upon the disclosure provided herein, that antibodies developed using a region that is conserved among one or more RELM molecule can be used to produce antibodies that react specifically with one or more RELM molecule. Methods for producing antibodies that specifically bind with a conserved protein domain which may otherwise be less immunogenic than other portions of the protein are well-known in the art and include, but are not limited to, conjugating the protein fragment of interest to a molecule (e.g., keyhole limpet hemocyanin, and the like), thereby rendering the protein domain immunogenic, or by the use of adjuvants (e.g., Freund's complete and/or incomplete adjuvant, and the like), or both. Thus, the invention encompasses antibodies that recognize at least one RELM and antibodies that specifically bind with more than one RELM, including antibodies that specifically bind with all RELMs.

One skilled in the art would appreciate, based upon the disclosure provided herein, which portions of RELM are less homologous with other proteins sharing conserved domains. However, the present invention is not limited to any particular domain; instead, the skilled artisan would understand that other non-conserved regions of the RELM proteins of the invention can be used to produce the antibodies of the invention as disclosed herein.

Therefore, the skilled artisan would appreciate, based upon the disclosure provided herein, that the present invention encompasses antibodies that neutralize and/or inhibit RELM activity (e.g., by inhibiting necessary RELM receptor/ligand interactions) which antibodies can recognize one or more RELMs, including, but not limited to, RELMβ and RELMα, as well as RELMs from various species (e.g., mouse, human, and/or rat RELMβ and/or RELMα).

One skilled in the art would also understand, based upon the disclosure provided herein, that it may be advantageous to inhibit the activity and/or expression of one type of RELM molecule without affecting the activity and/or expression of other RELM molecules. For example, it may be beneficial to inhibit RELMα expression to treat colon cancer where RELMβ is over-expressed in colon tissue, while not inhibiting the expression and/or activity of RELMα in adipose, tongue, mammary and/or lung tissue where the existing level of RELMα in the adipose, tongue, mammary and/or lung tissue is necessary for continued proper functioning of cellular processes in that tissue. Thus, whether inhibition of RELM expression and/or activity is achieved using antibodies, antisense nucleic acids, and the like, one skilled in the art would appreciate, based upon the disclosure provided herein, that the present invention encompasses selectively affecting one or more RELM molecules and, in certain cases, the invention encompasses inhibiting the expression or activity of all RELMs. Whether one or more RELMs should be affected can be readily determined by the skilled artisan based on which disease, disorder or condition is being treated, and the specific tissue (e.g., colon, breast, lung, tongue, and adipose) being targeted.

The invention should not be construed as being limited solely to the antibodies disclosed herein or to any particular immunogenic portion of the proteins of the invention. Rather, the invention should be construed to include other antibodies, as that term is defined elsewhere herein, to RELM, or portions thereof, or to proteins sharing at least about 30% homology with a polypeptide having the amino acid sequence of at least one of SEQ ID NO:2, SEQ ID NO:4, SEQ ID NO:6, SEQ ID NO:13, and SEQ ID NO:8. Preferably, the polypeptide is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to at least one of mouse RELMβ (SEQ ID NO:2), human RELMβ (SEQ ID NO:4), mouse RELMα (SEQ ID NO:6), alternately translated mouse RELMα (SEQ ID NO: 13), and rat RELMα (SEQ ID NO:8). More preferably, the polypeptide that specifically binds with an antibody specific for mammalian RELM is at least one of mouse RELMβ, human RELMβ, mouse RELMα, and rat RELMα. Most preferably, the polypeptide that specifically binds with an antibody that specifically binds with a mammalian RELM is at least one of SEQ ID NO: 2, SEQ ID NO:4, SEQ ID NO:6, SEQ ID NO: 13, and SEQ ID NO:8.

The invention encompasses polyclonal, monoclonal, synthetic antibodies, and the like. One skilled in the art would understand, based upon the disclosure provided herein, that the crucial feature of the antibody of the invention is that the antibody bind specifically with RELM. That is, the antibody of the invention recognizes RELM, or a fragment thereof (e.g., an immunogenic portion or antigenic determinant thereof), on Western blots, in immunostaining of cells, and immunoprecipitates RELM using standard methods well-known in the art.

One skilled in the art would appreciate, based upon the disclosure provided herein, that the antibodies can be used to localize the relevant protein in a cell and to study the role(s) of the antigen recognized thereby in cell processes. Moreover, the antibodies can be used to detect and or measure the amount of protein present in a biological sample using well-known methods such as, but not limited to, Western blotting and enzyme-linked immunosorbent assay (ELISA). Moreover, the antibodies can be used to immunoprecipitate and/or immuno-affinity purify their cognate antigen using methods well-known in the art. In addition, the antibody can be used to decrease the level of RELM in a cell thereby inhibiting the effect(s) of RELM in a cell. Thus, by administering the antibody to a cell or to the tissues of an animal or to the animal itself, the required RELM receptor/ligand interactions are therefore inhibited such that the effect of RELM mediated signaling are also inhibited. One skilled in the art would understand, based upon the disclosure provided herein, that detectable effects upon inhibiting RELM ligand/receptor interaction using an anti-RELM antibody can include, but are not limited to, decreased proliferation of intestinal epithelial cells, reduced inflammation, improvement in stool consistency, and the like.

One skilled in the art would appreciate, based upon the disclosure provided herein, that the invention encompasses administering an antibody that specifically binds with RELMP orally, parenterally, or both, to inhibit RELMβ function in the intestinal lumen and/or in the circulation, respectively.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein.

Further, the antibody of the invention may be “humanized” using the technology described in, for example, Wright et al. (supra), and in the references cited therein, and in Gu et al. (1997, Thrombosis and Hematocyst 77:755-759), and other methods of humanizing antibodies well-known in the art or to be developed.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruifet al. 1995, J. Mol. Biol. 248:97-105).

VII. Compositions

The invention includes a composition comprising an isolated nucleic acid complementary to a nucleic acid, or a portion thereof, encoding a mammalian RELM which is in an antisense orientation with respect to transcription. Preferably, the composition comprises a pharmaceutically acceptable carrier. Preferably, the antisense nucleic acid is complementary with a nucleic acid having at least about 30% homology with at least one of SEQ ID NO:1, SEQ ID NO:3, SEQ ID NO:5, and SEQ ID NO:7, or a fragment thereof. Preferably, the nucleic acid is about 35% homologous, more preferably, about 35% homologous, more preferably, about 40% homologous, even more preferably, about 45% homologous, more preferably, about 50% homologous, preferably, about 55% homologous, more preferably, about 60% homologous, even more preferably, about 65% homologous, more preferably, about 70% homologous, even more preferably, about 75% homologous, preferably, about 80% homologous, more preferably, about 85% homologous, even more preferably, about 90% homologous, and most preferably, about 95% homologous to a nucleic acid complementary to a portion or all of a nucleic acid encoding a mammalian RELM having the sequence of at least one of SEQ ID NO: 1, SEQ ID NO:3, SEQ ID NO:5, and SEQ ID NO:7, or a fragment thereof, which is in an antisense orientation with respect to transcription. Most preferably, the nucleic acid is complementary to a portion or all of a nucleic acid that is at least one of SEQ ID NO:1, SEQ ID NO:3, SEQ ID NO:5, and SEQ ID NO:7, or a fragment thereof. Such antisense nucleic acid serves to, inter alia, inhibit the expression, function, or both, of a resistin-like molecule.

The invention includes a composition comprising an isolated mammalian RELM polypeptide as described herein. Preferably, the composition comprises a pharmaceutically-acceptable carrier. Further, preferably, the isolated polypeptide comprising a mammalian RELM is at least about 30% homologous to a polypeptide having the amino acid sequence of at least one of SEQ ID NO:2, SEQ ID NO:4, SEQ ID NO:6, SEQ ID NO:8, and SEQ ID NO:13. Preferably, the isolated polypeptide is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to at least one of SEQ ID NO:2, SEQ ID NO:4, SEQ ID NO:6, SEQ ID NO:8, and SEQIDNO:13. More preferably, the isolated polypeptide comprising a mammalian RELM is at least one of mouse RELM, rat RELM, and human RELM. Most preferably, the isolated polypeptide comprising a mammalian resistin-like molecule is at least one of SEQ ID NO:2, SEQ ID NO:4, SEQ ID NO:6, SEQ ID NO:8, and SEQ ID NO:13.

The invention also includes a composition comprising an antibody that specifically binds RELM. Preferably, the composition comprises a pharmaceutically-acceptable carrier. Preferably, the antibody that specifically binds with RELM specifically binds a protein, or portion thereof, sharing at least about 30% homology with a polypeptide having the amino acid sequence of at least one of SEQ ID NO:2, SEQ ID NO:4, SEQ ID NO:6, SEQ ID NO:13, and SEQ ID NO:8. Preferably, the polypeptide is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to at least one of mouse RELMβ (SEQ ID NO:2), human RELMβ (SEQ ID NO:4), mouse RELMα (SEQ ID NO:6), alternately translated mouse RELMα (SEQ ID NO: 13), and rat RELMα (SEQ ID NO:8). More preferably, the polypeptide that specifically binds with an antibody specific for mammalian RELM is at least one of mouse RELMβ, human RELMβ, mouse RELMα, and rat RELMα. Most preferably, the polypeptide that specifically binds with an antibody that specifically binds with a mammalian RELM is at least one of SEQ ID NO: 2, SEQ ID NO:4, SEQ ID NO:6, SEQ ID NO:13, and SEQ ID NO:8.

The invention further includes a composition comprising an isolated nucleic acid encoding a mammalian RELM. Preferably, the composition comprises a pharmaceutically acceptable carrier. Further, the nucleic acid encoding a mammalian RELM shares at least about 30% identity with at least one nucleic acid having the sequence of (SEQ ID NO:1), (SEQ ID NO:3), (SEQ ID NO:5), and (SEQ ID NO:7). Preferably, the nucleic acid is about 35% homologous, more preferably, about 40% homologous, more preferably, about 45% homologous, even more preferably, about 50% homologous, more preferably, about 55% homologous, preferably, about 60% homologous, more preferably, about 65% homologous, even more preferably, about 70% homologous, more preferably, about 75% homologous, even more preferably, about 80% homologous, preferably, about 85% homologous, more preferably, about 90% homologous, even more preferably, about 95% homologous, and most preferably, about 99% homologous to at least one of SEQ ID NO:1, SEQ ID NO:3, SEQ ID NO:5, and SEQ ID NO:7 disclosed herein. Even more preferably, the nucleic acid is at least one of SEQ ID NO:1, SEQ ID NO:3, SEQ ID NO:5, and SEQ ID NO:7.

The compositions can be used to administer RELM to a cell, a tissue, or an animal or to inhibit expression of RELM in a cell, a tissue, or an animal. The compositions are useful to treat a disease, disorder or condition mediated by altered expression of RELM such that decreasing or increasing RELM expression or the level of the protein in a cell, tissue, or animal, is beneficial to the animal. That is, where a disease, disorder or condition in an animal is mediated by or associate with altered level of RELM expression or protein level, the composition can be used to modulate such expression or protein level of RELM.

For administration to the mammal, a polypeptide, or a nucleic acid encoding it, and/or an antisense nucleic acid complementary to all or a portion thereof, can be suspended in any pharmaceutically acceptable carrier, for example, HEPES buffered saline at a pH of about 7.8.

Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable, carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer RELM and/or a nucleic acid encoding the same according to the methods of the invention.

Compounds which are identified using any of the methods described herein may be formulated and administered to a mammal for treatment of intestinal (e.g., colonic) tumors, colon cancer, familial adenomatous polyposis, irritable bowel disease, inflammatory bowel disease, breast cancer, tongue cancer, diabetes, insulin resistance, and obesity, are now described.

The invention encompasses the preparation and use of pharmaceutical compositions comprising a compound useful for treatment of intestinal (e.g., colonic) tumors, colon cancer, familial adenomatous polyposis, irritable bowel disease, inflammatory bowel disease, breast cancer, tongue cancer, diabetes, insulin resistance, and obesity as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para- hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may firther comprise buffering agents, salts, or one or more other of the additional ingredients described herein. Other ophthalmalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

VIII. Methods

A. Methods of identifying useful compounds

The present invention further includes a method of identifying a compound that affects expression of RELM in a cell. The method comprises contacting a cell with a test compound and comparing the level of expression of RELM in the cell so contacted with the level of expression of RELM in an otherwise identical cell not contacted with the compound. If the level of expression of RELM is higher or lower in the cell contacted with the test compound compared to the level of expression of RELM in the otherwise identical cell not contacted with the test compound, this is an indication that the test compound affects expression of RELM in a cell.

The invention encompasses methods to identify a compound that affects expression of RELMα, RELMβ, or both. One skilled in the art would appreciate, based upon the disclosure provided herein, that assessing the level of RELMα, RELMβ, or both, can be performed using probes (e.g., antibodies and/or nucleic acid probes that specifically bind with of RELMα, RELMβ, or both), such that the method can identify a compound that selectively affects expression of RELMα, RELMβ, or both. Such compounds are useful for inhibiting expression of one RELM family member while not affecting the level of another RELM family member. One skilled in the art would understand that such compounds can be useful for inhibiting a disease, disorder, or condition mediated by and/or associated with increased expression of RELM (e.g., increased level of RELMβ is associated with colon cancer, inflammatory bowel disease) and RELMα expression is associated with insurance tolerance, diabetes, Syndrome X, and obesity. Thus, the skilled artisan would appreciate, based on the disclosure provided herein, that it may useful to decrease expression of RELMα or β while leaving the expression of the other RELM either untouched or increased.

Similarly, the present invention includes a method of identifying a compound that reduces expression of RELM in a cell. The method comprises contacting a cell with a test compound and comparing the level of expression of RELM in the cell contacted with the compound with the level of expression of RELM in an otherwise identical cell, which is not contacted with the compound. If the level of expression of RELM is lower in the cell contacted with the compound compared to the level in the cell that was not contacted with the compound, then that is an indication that the test compound reduces expression of RELM in a cell.

One skilled in the art would understand, based upon the disclosure provided herein, that the invention encompasses identifying a compound that increases, inter alia, RELMα, RELMβ, or both. This is because the data disclosed herein demonstrate that there are certain diseases, disorders, or conditions that are associated with/mediated by increased levels of RELMβ (e.g., colon cancer, familial adenomatous polyposis, irritable bowel disease, inflammatory bowel disease, intestinal tumors, and the like) and certain diseases, disorders, or conditions associated with/mediated by increased RELMα (e.g., tongue cancer, breast cancer, diabetes, obesity, and the like). Therefore, methods of identifying a compound that increases the level of inter alia, RELMα, RELMβ, or both, are helpful for treating and/or alleviating diseases, disorders or conditions associated with decreased expression of RELMα, RELMβ, or both.

The invention also includes a method of identifying a compound that increases expression of RELM in a cell. The method comprises contacting a cell with a test compound and comparing the level of expression of RELM in the cell contacted with the compound with the level of expression of RELM in an otherwise identical cell, which is not contacted with the compound. If the level of expression of RELM is higher in the cell contacted with the compound compared to the level in the cell that was not contacted with the compound, then that is an indication that the test compound increases expression of RELM in a cell.

A compound that increases RELM is a cell is useful since it has been demonstrated herein that TZDs increase the level of RELMα and -β in adipose tissue and in colon, respectively. Additionally, the high degree of identity between the sequence of resistin and the sequence of RELMα and -β indicates that RELMs are associated with glucose metabolism and with the powerful antidiabetic TZDs that increase the level of RELMα and -β.

TZDs have powerful beneficial effects in treating/alleviating, e.g., diabetes and inflammatory bowel disease. Thus, since TZDs increase the level of RELM α and RELMβ, and since it is known that intestinal bacteria are a causative agent of such diseases as inflammatory bowel disease, which is responsive to treatment with TZDs, one skilled in the art would appreciate, based upon the disclosure provided herein, that a compound, besides a TZD, that increases RELM expression can provide novel potential therapeutics for diabetes, insulin resistance, Syndrome X, colon cancer, familial adenomatous polyposis, irritable bowel disease, inflammatory bowel disease, intestinal tumors, breast cancer, tongue cancer, diabetes, insulin resistance, and obesity.

Thus, the skilled artisan would appreciate, based upon the disclosure provided herein, that increasing RELMα levels can be beneficial for diabetes, since endogenous RELMα expression increases in response to antidiabetic drugs. The skilled artisan would further appreciate, based upon the disclosure provided therein, that increasing RELMβ level is likely to be beneficial for intestinal disease since endogenous RELMβ expression is regulated by intestinal activity and bacterial exposure. Thus, methods of identifying a compound that increases the level of RELMα, RELMβ, or both, can be used to treat various diseases, disorders, or conditions.

One skilled in the art would appreciate, based on the disclosure provided herein, that the level of expression of RELM in the cell may be measured by determining the level of expression of mRNA encoding RELM. Alternatively, the level of expression of mRNA encoding RELM can be determined by using immunological methods to assess RELM production from such mRNA as exemplified herein using Western blot analysis using an anti-RELM antibody of the invention. Further, nucleic acid-based detection methods, such as Northern blot and PCR assays and the like, can be used as well. In addition, the level of RELM activity in a cell can also be assessed by determining the level of various parameters which can be affected by RELM activity such as, for example, the level of RELM-receptor binding, activation of tyrosine kinases, activation of serine/threonine kinases, activation of tyrosine phosphatases, activation of scrine phosphatases, alteration of intracellular calcium fluxes, alteration in intracellular cyclic AMP levels, alteration of intracellular cyclic GMP levels. Thus, one skilled in the art would appreciate, based upon the extensive disclosure and reduction to practice provided herein, that there are a plethora of methods that are well-known in the art, which can be used to asses the level of expression of RELM in a cell including those disclosed herein and others which may be developed in the future.

Further, one skilled in the art would appreciate based on the disclosure provided herein that, as disclosed in the examples below, a cell which lacks endogenous RELM expression can be transfected with a vector comprising an isolated nucleic acid encoding RELM whereby expression of RELM is effected in the cell. The transfected cell is then contacted with the test compound thereby allowing the determination of whether the compound affects the expression of RELM. Therefore, one skilled in the art armed with the present invention would be able to, by selectively transfecting a cell lacking detectable levels of RELM using RELM-expressing vectors, identify a compound which selectively affects RELM expression.

The invention includes a method of identifying a protein that specifically binds with RELM. That is, one skilled in the art would appreciate, based upon the disclosure provided herein, that RELMs, which are secreted from a cell, likely effect cell function by specifically binding with at least one protein, preferably a RELM receptor, another RELM molecule, and/or a RELM ligand. Thus, the invention encompasses methods, which are well-known in the art or to be developed, for identifying a protein that specifically binds with RELM. Such methods include, but are not limited to, protein binding assays wherein the target protein, i.e., RELM, is immobilized on an appropriate support and incubated under conditions that allow RELM binding with a RELM-associated protein. RELM can be immobilized on a support using standard methods such as, but not limited to, production of RELM comprising a glutathione-S-transferase (GST) tag, a maltose binding protein (MBP) tag, or a His₆-tag, where the fusion protein is then bound to glutathione-Sepharose beads, a maltose-column, or a nickel chelation resin (e.g., His-Bind resin, Novagen, Madison, Wis.), respectively. The solid support is washed to remove proteins which may be non-specifically bound thereto and any RELM-associated protein can then be dissociated from the matrix thereby identifying a RELM-associated protein.

In addition, a protein that specifically binds with RELM, e.g. a receptor or other RELM-associated protein, can be identified using, for example, a yeast two hybrid assay. Yeast two hybrid assay methods are well-known in the art and can be performed using commercially available kits (e.g., MATCHMAKER™ Systems, Clontech Laboratories, Inc., Palo Alto, Calif., and other such kits) according to standard methods. Therefore, once armed with the teachings provided herein, e.g., the full amino and nucleic acid sequences of the “bait” protein, RELM, one skilled in the art can easily identify a protein that specifically binds with RELM such as, but not limited to, a RELM receptor protein.

One skilled in the art would understand, based upon the disclosure provided herein, that the invention encompasses any molecule identified using the methods discussed elsewhere herein. That is, molecules that associate with RELM, such as but not limited to, a RELM receptor protein, can be used to develop therapeutics and diagnostics for diseases, disorders or conditions mediated by RELM interaction with a RELM-associated protein such as colon cancer, familial adenomatous polyposis, irritable bowel disease, inflammatory bowel disease, intestinal tumors, breast cancer, tongue cancer, diabetes, insulin resistance, and obesity. That is, one skilled in the art would appreciate, as more fully set forth elsewhere herein in -discussing antibodies that specifically bind with RELM, that a RELM-associated protein can be used to develop therapeutics that inhibit RELM activity in a cell by inhibiting necessary RELM receptor/ligand interactions and other RFLM binding interactions such as, e.g., RELM multimerization, which are required for RELM activity.

RELM-associated proteins identified by the above-disclosed methods can be used directly to inhibit RELM interactions by contacting a cell with the RELM-associated protein, or a portion thereof, or they can be used to develop antibodies and/or peptidomimetics that can inhibit the RFLM-associated interaction with RELM thereby inhibiting RELM function and activity. Thus, RELM-associated proteins, including a RELM receptor protein, are useful and are encompassed by the invention.

B. Methods of treating or alleviating a disease, disorder or condition associated with or mediated by RELM malexpression

One skilled in the art would understand, based upon the disclosure provided herein, that it may be useful to increase the level or activity of RELM in a cell. That is, it can be useful to treat or alleviate a disease, disorder of condition associated with or mediated by decreased expression, level, or activity of RELM by administering RELM. Such diseases, disorders or conditions include, but are not limited to, intestinal tumors, colon cancer, irritable bowel disease, inflammatory bowel disease, familial adenomatous polyposis, diabetes, insulin resistance, obesity, breast cancer, tongue cancer, lung cancer, and the like.

That is, one skilled in the art would understand, based upon the disclosure provided herein, that increased expression of RELM is associated with and/or can mediate a beneficial effect in a patient afflicted with colon cancer, familial adenomatous polyposis, irritable bowel disease, inflammatory bowel disease, intestinal tumors, breast cancer, tongue cancer, insulin resistance, syndrome X, type 2 diabetes, and obesity, among other things. Thus, the invention includes methods of treating such diseases, disorders, or conditions, by increasing RELM expression such as by, among other things, administering a RELM protein and/or a nucleic acid encoding a RELM protein which is expressed in a cell.

The skilled artisan would appreciate, based on the disclosure provided herein, that the data disclosed herein demonstrate that administration of TZDs (e.g., rosiglitazone) raised the level of RELMα in white adipose tissue (e.g., in stromovascular cells thereof) suggesting that the beneficial effect(s) of TZDs is mediated by/associated with expression of RELMα. Indeed, it has been discovered that administration of TZD to ob/ob mice, an art-recognized model of obesity and type 2 diabetes, increased the level of RELMα expression. These data indicate that RELMα plays a role in the glucose-lowering effect(s) of TZDs and related drugs that target the nuclear receptor PPARy. Thus, the present invention includes methods of treatment/alleviation of various diseases, disorders, or conditions by increasing the level of RELMα, e.g., by raising the level of RELMα mRNA, by increasing the level of RELMα protein, or both, since increased levels of RELMα are associated with administration and beneficial effects of the powerful therapeutics, TZDs.

Similarly, one skilled in the art would understand, based upon the disclosure provided herein that increased expression of RELMβ is associated with and/or can mediate a beneficial effect in a patient afflicted with colon cancer, familial adenomatous polyposis, irritable bowel disease, inflammatory bowel disease, intestinal tumors, breast cancer, tongue cancer, diabetes, insulin resistance, and obesity, among other things, increased RELMβ expression can be useful for treating such diseases, disorders, or conditions.

More specifically, this is because the data disclosed herein demonstrate that administration of TZDs (e.g., rosiglitazone), which improves inflammatory bowel disease, raised the level of RELMβ in colon, suggesting that the beneficial effect(s) of TZDs is mediated by/associated with increased expression of RELMβ. Indeed, it has been discovered that TZD increased the level of RELMβ expression in a human intestinal cell line (LS124T) in vitro. These data also indicate that RELMβ plays a role in the glucose-lowering effect(s) of TZDs and related drugs that target the nuclear receptor PPARγ since administration of rosiglitazone increased RELMβ expression in a dose-dependent manner. These data indicate that the effect of TZDs is associated with and/or mediated by an increase in RELMβ.

Further, mice raised in germ-free conditions demonstrate decreased expression of RELMβ compared with RELM β expression in mice raised in normal conditions. Since it is well-known that intestinal bacteria play a causative role inflammatory bowel disease, the decrease in RELMβ expression in germ-free mice indicates that RELMβ is induced in tissues comprising intestinal bacteria because RELMβ provides a beneficial effect in such tissues. Thus, the present invention includes methods of treatment/alleviation of various diseases, disorders, or conditions by increasing the level of RELMβ, e.g., by raising the level of RELMβ mRNA, by increasing the level of RELMβ protein, or both, since increased levels of RELMβ are associated with beneficial effects of the powerful therapeutics, TZDs, and increased level of RELMβ is also associated with and/or plays a role in inflammatory bowel disease which is treatable by administration of TZDs.

The invention includes a method of alleviating a disease, disorder or condition mediated by malexpression of RELM. Where the disease, disorder or condition is associated with over-expression of RELM, the method comprises administering an antisense nucleic acid complementary to a nucleic acid encoding RELM to a patient afflicted with a disease, disorder or condition mediated by increased RELM expression compared to the level of RELM expression in otherwise identical but normal tissue, i.e., tissue which does not exhibit any detectable clinical parameters associated with the disease, disorder or condition being treated or alleviated. This, in turn, mediates a decrease in RELM expression thereby alleviating a disease, disorder or condition mediated by malexpression of RELM. Such diseases, disorder or conditions include, but are not limited to, colon cancer, familial adenomatous polyposis, irritable bowel disease, inflammatory bowel disease, intestinal tumors, breast cancer, tongue cancer, diabetes, insulin resistance, Syndrome X, and obesity.

More specifically, one skilled in the art would understand, based upon the disclosure provided herein, that the invention encompasses a method of treating a disease mediated by increased RELMα, RELMβ, or both, or by decreased RELMα, RELMβ, or both. This is because the data disclosed herein demonstrate that there are certain diseases, disorders, or conditions that are associated with/mediated by increased levels of RELMβ (e.g., colon cancer, familial adenomatous polyposis, irritable bowel disease, inflammatory bowel disease, intestinal tumors, and the like) and certain diseases, disorders, or conditions associated with/mediated by increased RELMα (e.g., tongue cancer, breast cancer, diabetes, Syndrome X, obesity, and the like). Further, the data disclosed herein indicate that RELMα and RELMβ are induced by the powerful antidiabetic drugs—TZDs—which are also useful for treatment of inflammatory bowel disease. Thus, conditions associated with or mediated by decreased levels of RELMα, RELMβ can be treated by increasing the level of RELMα, RELMβ, or both. Therefore, methods of identifying a compound that increases the level of inter alia, RELMα, RELMβ, or both, are helpful for treating and/or alleviating diseases, disorders or conditions associated with decreased expression of RELMα, RELMβ, or both.

Antisense nucleic acids that inhibit expression of RELM can therefore also be used for the manufacture of a medicament for treatment of a disease, disorder or condition mediated by increased expression of RELM when compared with expression of RELM in a cell and/or a patient not afflicted with the disease, disorder or condition.

This is because, as disclosed elsewhere herein, increased expression of RELMβ is associated with abnormal cell proliferation associated with colon cancer, familial adenomatous polyposis, irritable bowel disease, inflammatory bowel disease, intestinal tumors, breast cancer, and tongue cancer. Thus, one skilled in the art would appreciate, based upon the disclosure provided herein, that inhibition of RELMβ expression can inhibit the deleterious effects of RELMβ malexpression.

Similarly, one skilled in the art would appreciate, based upon the disclosure provided elsewhere herein, that inhibition of RELMα expression can inhibit diseases, disorders or conditions mediated by malexpression of RELMα. Given the sequence homology between RELMα and resistin, the fact that both proteins are secreted and are expressed in white adipose tissue, and given that resistin is an important protein involved in glucose resistance, RELMα likely plays an important role in diseases, disorders or conditions associated with glucose metabolism, such as, but not limited to, obesity, diabetes, insulin resistance, Syndrome X, and polycystic ovary disease. Further, inhibition of RELMα expression and/or activity can be useful in treating diseases specific to tissues where RELMα is expressed such as tongue, mammary, white adipose and lung tissues. Therefore, inhibiting expression of RELMα is useful for treating diseases, disorders, or conditions associated with glucose metabolism as disclosed in the PCT application having International Publication No. WO 00/64920, which is incorporated by reference herein in its entirety, as well as for treating tongue cancer, lung cancer, breast cancer, and the like.

One skilled in the art would understand, based upon the disclosure provided herein, that since reduced RELM expression can mediate a beneficial effect, methods of decreasing expression of RELM, decreasing the level of RELM polypeptide present in the cell, and/or decreasing the activity of RELM in a cell (using, e.g., antisense nucleic acids, ribozymes, antibodies, and the like), can be used to treat and/or alleviate a disease, disorder or condition associated with altered expression of RELM where a lower level of RELM would provide a benefit. Thus, whether an antisense nucleic acid or a blocking antibody is administered, the crucial feature of the present invention is that the expression of RELM be reduced in a cell.

Techniques for inhibiting expression of a nucleic acid in a cell are well known in the art and encompass such methods as disclosed herein (e.g., inhibition using an antibody, an antisense nucleic acid, and the like). Other techniques useful for inhibiting expression of a nucleic acid encoding RELM include, but are not limited to, using nucleotide reagents that target specific sequences of the RELM promoter, and the like.

Whether expression of RELM, levels of the polypeptide, or its activity, is increased or decreased, one skilled in the art would appreciate, based on this disclosure, that methods of reducing or inducing RELM of the invention encompass administering a recombinant cell that either expresses or lacks expression of RELM.

In another embodiment of the invention, an individual suffering from a disease, disorder or a condition that is associated with or mediated by RELM expression can be treated by supplementing, augmenting and/or replacing defective cells with cells that lack RELM expression. The cells can be derived from cells obtained from a normal syngeneic matched donor or cells obtained from the individual to be treated. The cells may be genetically modified to inhibit RELM expression.

An example of a disease, disorder or a condition associated with or mediated by RELM expression is colon cancer, familial adenomatous polyposis, irritable bowel disease, inflammatory bowel disease, intestinal tumors, breast cancer, tongue cancer, diabetes, insulin resistance, obesity, and the like.

In addition to replacing defective cells with repaired cells or normal cells from matched donors, the method of the invention may also be used to facilitate expression of a desired protein that when secreted in the an animal, has a beneficial effect. That is, cells may be isolated, furnished with a gene encoding RELM and introduced into the donor or into a syngeneic matched recipient. Expression of the RELM exerts a therapeutic effect.

This aspect of the invention relates to gene therapy in which therapeutic amounts of RELM are administered to an individual.

According to some aspects of the present invention, recombinant cells transfected with either nucleic acid encoding RELM, antisense nucleic acids or a knock-out targeting vector of the invention, can be used as cell therapeutics to treat a disease, disorder or a condition characterized by expression of RELM or the lack thereof.

In particular, a gene construct that comprises a heterologous gene which encodes RELM is introduced into cells. These recombinant cells are used to purify isolated RELM, which was then administered to an animal causing diabetic-levels of blood sugar. One skilled in the art would understand, based upon the disclosure provided herein, that instead of administering an isolated RELM polypeptide, RELM can be administered to a mammal in need thereof by administering to the mammal the recombinant cells themselves. This will benefit the recipient individual who will benefit when the protein is expressed and secreted by the recombinant cell into the recipient's system.

According to the present invention, gene constructs comprising nucleotide sequences of the invention are introduced into cells. That is, the cells, referred to herein as “recombinant cells,” are genetically altered to introduce a nucleic acid encoding RELM or a nucleic acid that inhibits RELM expression in and/or secretion by the recombinant cell thereby mediating a beneficial effect on an recipient to which the recombinant cell is administered. According to some aspects of the invention, cells obtained from the same individual to be treated or from another individual, or from a non-human animal, can be genetically altered to replace a defective gene and/or to introduce a gene whose expression has a beneficial effect on the individual or to inhibit RELM expression which can have a beneficial effect on the individual.

In some aspects of the invention, an individual suffering from a disease, disorder or a condition can be treated by supplementing, augmenting and/or replacing defective or deficient nucleic acid encoding RELM by providing an isolated recombinant cells containing gene constructs that include normal, functioning copies of a nucleic acid encoding RELM. This aspect of the invention relates to gene therapy in which the individual is provided with a nucleic encoding RELM for which they are deficient in presence and/or function. The isolated nucleic acid encoding RELM provided by the cell compensates for the defective RELM expression of the individual, because, when the nucleic acid is expressed in the individual, a protein is produced which serves to alleviate or otherwise treat the disease, disorder or condition in the individual. Such nucleic acid preferably encodes a RELM polypeptide that is secreted from the recombinant cell.

In all cases in which a gene construct encoding RELM is transfected into a cell, the nucleic acid is operably linked to an appropriate promoter/regulatory sequence which is required to achieve expression of the nucleic acid in the recombinant cell. Such promoter/regulatory sequences include but are not limited to, constitutive and inducible and/or tissue specific and differentiation specific promoters, and are discussed elsewhere herein. Constitutive promoters include, but are not limited to, the cytomegalovirus immediate early promoter and the Rous sarcoma virus promoter. In addition, housekeeping promoters such as those which regulate expression of housekeeping genes may also be used. Other promoters include those which are preferentially expressed in cells of the central nervous system, such as, but not limited the promoter for the gene encoding glial fibrillary acidic protein. In addition, promoter/regulatory elements may be selected such that gene expression is inducible. For example, a tetracycline inducible promoter may be used (Freundlich et al., 1997, Meth. Enzymol. 283:159-173).

The gene construct is preferably provided as an expression vector which includes the coding sequence of a mammalian RELM of the invention operably linked to essential promoter/regulatory sequences such that when the vector is transfected into the cell, the coding sequence is expressed by the cell. The coding sequence is operably linked to the promoter/regulatory elements necessary for expression of the sequence in the cells. The nucleotide sequence that encodes the protein may be cDNA, genomic DNA, synthesized DNA or a hybrid thereof or an RNA molecule such as mRNA.

The gene construct, which includes the nucleotide sequence encoding RELM operably linked to the promoter/regulatory elements, may remain present in the cell as a functioning episomal molecule or it may integrate into the chromosomal DNA of the cell. Genetic material maybe introduced into cells where it remains as separate genetic material in the form of a plasmid. Alternatively, linear DNA which can integrate into a host cell chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

In order for genetic material in an expression vector to be expressed, the promoter/regulatory elements must be operably linked to the nucleotide sequence that encodes the protein. In order to maximize protein production, promoter/regulatory sequences may be selected which are well suited for gene expression in the desired cells. Moreover, codons may be selected which are most efficiently transcribed in the cell. One having ordinary skill in the art can produce recombinant genetic material as expression vectors which are finctional in the desired cells.

It is also contemplated that promoter/regulatory elements may be selected to facilitate tissue specific expression of the protein. Thus, for example, specific promoter/regulatory sequences may be provided such that the heterologous gene will only be expressed in the tissue where the recombinant cells are implanted. One skilled in the art would understand, based upon the disclosure provided herein, that the preferred tissues where the expression or lack of expression of RELM is to be targeted include, but are not limited to, white adipose tissue, brown adipose tissue, blood, hepatic tissue, and skeletal muscle. In addition, promoter/regulatory elements may be selected such that gene expression is inducible. For example, a tetracycline inducible promoter may be used (Freundlich et al., 1997, Meth. Enzymol. 283:159-173).

The nucleic acid encoding RELM preferably includes a signal sequence as disclosed elsewhere herein (e.g., amino acids 1 to 20 of mouse RELMβ (SEQ ID NO:2) and amino acids 1 to 23 or 24 of mouse RELMα (SEQ ID NO:6), which directs the transport and secretion of the RELM encoded by the isolated nucleic acid in the recombinant cell. The signal sequence is generally processed and removed upon secretion of the mature RELM protein from the cell.

In addition to providing cells with recombinant genetic material that either corrects a genetic defect in the cells, that encodes a protein which is otherwise not present in sufficient quantities and/or finctional condition so that the genetic material corrects a genetic defect in the individual, and/or that encodes a protein which is useful as beneficial in the treatment or prevention of a particular disease, disorder or condition associated therewith, and that inhibits expression of RELM in the cell (e.g., a knock-out targeting vector, an antisense nucleic acid, and the like), genetic material can also be introduced into the recombinant cells used in the present invention to provide a means for selectively terminating such cells should such termination become desirable. Such means for targeting recombinant cells for destruction may be introduced into recombinant cells.

According to the invention, recombinant cells can be furnished with genetic material which renders them specifically susceptible to destruction. For example, recombinant cells may be provided with a gene that encodes a receptor that can be specifically targeted with a cytotoxic agent. An expressible form of a gene that can be used to induce selective cell death can be introduced into the recombinant cells. In such a system, cells expressing the protein encoded by the gene are susceptible to targeted killing under specific conditions or in, the presence or absence of specific agents. For example, an expressible form of a herpes virus thymidine kinase (herpes tk) gene can be introduced into the recombinant cells and used to induce selective cell death. When the introduced genetic material that includes the herpes tk gene is introduced into the individual, herpes tk will be produced. If it is desirable or necessary to kill the implanted recombinant cells, the drug gangcyclovir can be administered to the individual which will cause the selective killing of any cell producing herpes tk. Thus, a system can be provided which allows for the selective destruction of implanted recombinant cells.

One skilled in the art would understand, based upon the disclosure provided herein, that the present invention encompasses production of recombinant cells to either provide RELM to or inhibit RELM expression in a mammal. That is, the cells can be used to administer RELM to an animal or to deliver a molecule (e.g., a knock-out targeting vector, an antisense nucleic acid, a ribozyme, and antibody that specifically binds with RELM, and the like).

Administration of RELM to an animal can be used as a model system to study the mechanism of action of RELM or to develop model systems useful for the development of diagnostics and/or therapeutics for diseases, disorders or conditions associated with RELM expression.

Further, the delivery of RELM to an animal mediated by administration of recombinant cells expressing and secreting RELM can also be used to treat or alleviate a disease, disorder or condition where increasing the level of RELM mediates a therapeutic effect. More specifically, administration of RELM to an animal by administering a recombinant cell expressing a nucleic acid encoding RELM can be useful for treatment of intestinal tumors, colon cancer, irritable bowel disease, inflammatory bowel disease, familial adenomatous polyposis, diabetes, insulin resistance, obesity, breast cancer, tongue cancer, lung cancer, among other things.

Alternatively, administration of recombinant cells comprising a nucleic acid the expression of which inhibits or reduces RELM expression, activity, and/or secretion from a cell, can be used as a model for the development of diagnostics and/or therapeutics useful for diseases, disorders or conditions associated with or mediated by RELM expression, activity, and/or secretion. The present invention encompasses that the recombinant cells can produce the molecule that inhibits RELM expression thereby providing such molecule to the animal. Alternatively, without wishing to be bound by any particular theory, the recombinant cells themselves, which are otherwise functional cells, except for the inability to express RELM, can perform the functions of otherwise identical but non-recombinant cells, without being subject to the RELM signaling pathway.

Cells, both obtained from an animal, from established cell lines that are commercially available or to be developed, or primary cells cultured in vitro, can be transfected using well known techniques readily available to those having ordinary skill in the art. Thus, the present invention is not limited to obtaining cells from a donor animal or from the patient animal itself. Rather, the invention includes using any cell that can be engineered using a nucleic acid of the invention such that the recombinant cell either expresses RELM (where it did not express RELM prior to being engineered, or where the cell produced RELM an a different level prior to the introduction of the nucleic acid into the cell) or the recombinant cell does not express RELM or expresses it at a lower level (where it expressed RELM before or expressed RELM at a different level prior to introduction of the nucleic acid into the cell).

Nucleic acids can be introduced into the cells using standard methods which are employed for introducing a gene construct into cells which express the protein encoded by the gene or which express a molecule that inhibits RELM expression. In some embodiments, cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some embodiments, recombinant adenovirus vectors are used to introduce DNA having a desired sequence into the cell. In some embodiments, recombinant retrovirus vectors are used to introduce DNA having a desired sequence into the cell. In some embodiments, standard calcium phosphate, DEAE dextran or lipid carrier mediated transfection techniques are employed to incorporate a desired DNA into dividing cells. Standard antibiotic resistance selection techniques can be used to identify and select transfected cells. In some embodiments, DNA is introduced directly into cells by microinjection. Similarly, well known electroporation or particle bombardment techniques can be used to introduce foreign DNA into cells. A second gene is usually co-transfected with and/or covalently linked to the nucleic acid encoding RELM, or knock-out targeting vector or antisense molecule thereto. The second gene is frequently a selectable antibiotic-resistance gene. Transfected recombinant cells can be selected by growing the cells in an antibiotic that kills cells that do not take up the selectable gene. In most cases where the two genes are unlinked and co-transfected, the cells that survive the antibiotic treatment contain and express both genes.

Where an isolated RELM polypeptide, an antibody that specifically binds with RELM, a RELM antisense nucleic acid, and/or recombinant cells of the invention are administered to an animal either to increase or reduce the level of RELM present in the animal, one skilled in the art would understand, based upon the disclosure provided herein, that the amount of the polypeptide, nucleic acid, antibody, or cell to be administered to the animal can be titrated by assessing the level of RELM and/or sugar present in the blood or by determining the level of expression of RELM or the level of RELM polypeptide or nucleic acid encoding RELM present in the tissues of the animal.

Methods for assessing the level of RELM (e.g., using anti-RELM antibodies in Western blot or other immune-based analyses such as ELISA) and/or methods for assessing the level of RELM expression in a cell and/or tissues (e.g., using Northern blot analysis, and the like) are disclosed herein or are well known to those skilled in the art. Such assays can be used to determine the “effective amount” of RELM, nucleic acid, antibody, antisense nucleic acid, ribozyme, recombinant cell, and the like, to be administered to the animal in order to reduce or increase the level of RELM and/or blood sugar amount to a desired level.

C. Methods of diagnosis and assessment of therapies

The present invention includes methods of diagnosis certain diseases, disorders, or conditions (e.g., colon cancer, familial adenomatous polyposis, irritable bowel disease, inflammatory bowel disease, intestinal tumors, breast cancer, tongue cancer, diabetes, insulin resistance, and obesity) which are associated with or mediated by malexpression of RELM.

The invention includes a method of diagnosing a colon tumor in an animal. The method comprises obtaining a biological sample from a first animal and comparing the level of RELMβ (expression, amount, activity) in that sample with the level of RELMβ in a sample obtained from a normal second animal that is otherwise identical to the first animal but which is not afflicted with a colon tumor. A higher level of RELMβ in the sample from the first animal compared with the level of RELMβ in the sample obtained from the second otherwise identical animal not afflicted with a colon tumor is an indication that the firs animal is afflicted with a colon tumor. This is because, as disclosed elsewhere herein, an increased level of RELMβ expression is associated with abnormal intestinal cell growth and tumor growth and development.

In one aspect, the biological sample is selected from the group consisting of a blood sample, a stool sample, a colon tissue biopsy, a cerebrospinal fluid sample, a lung biopsy, a fat biopsy, and the like.

Likewise, the invention includes a method of diagnosing familial adenomatous polyposis in an animal. The method comprises obtaining a sample from a first animal and comparing the level of RELMβ (expression, amount, activity) with the level of RELMβ in a sample from a like second animal not afflicted with familial adenomatous polyposis. A higher level of RELMβ in the sample from the first animal compared with the level of RELMβ in the second like animal is an indication that the first animal is afflicted with familial adenomatous polyposis. This is because, as disclosed previously elsewhere herein, an increased level of RELMβ is correlated with familial adenomatous polyposis in an art-recognized mouse model of this disease (i.e., the min transgenic mouse model). Thus, one skilled in the art would appreciate, based upon the disclosure provided herein, that a higher level of RELMβ in a sample indicates that the source of the sample is afflicted with a disease, disorder or condition associated with increased RELMβ expression such as, but not limited to, colon cancer, familial adenomatous polyposis, irritable bowel disease, inflammatory bowel disease, and intestinal tumors.

The invention includes a method of diagnosing inflammatory bowel disease in an animal. The method comprises obtaining a biological sample from an animal, assessing the level of resistin-like molecule β in the sample, and comparing the level of resistin-like molecule β in the sample with the level of resistin-like molecule β in a biological sample obtained from a second otherwise identical animal which is known not to be afflicted with inflammatory bowel disease. A higher level of resistin-like molecule β in the biological sample from the first animal compared with the level of resistin-like molecule β in the biological sample from the second otherwise identical animal is an indication that the first animal is afflicted with inflammatory bowel disease. Thus, diagnosing inflammatory bowel disease in an animal. This is because, as more fully disclosed elsewhere herein, increased expression of RELMβ is associated with inflammatory bowel disease in that, inter alia, mice raised in germ-free conditions have decreased RELMβ compared with mice raised under normal conditions which have bacterial in their intestines. Further, data disclosed herein demonstrate that RELMβ expression is associated with intestinal epithelial cell proliferation.

The invention includes a method of diagnosing insulin resistance in an animal. The method comprises obtaining a biological sample from a first animal, assessing the level of resistin-like molecule α in the sample, and comparing the level of resistin-like molecule a in the sample with the level of resistin-like molecule α in a biological sample obtained from a second otherwise identical animal that is not afflicted with insulin resistance. A higher level of resistin-like molecule α in the sample from the first animal compared with the level of resistin-like molecule a in the sample from the second otherwise identical animal is an indication that the first animal is afflicted with insulin resistance.

This is because, as disclosed elsewhere herein, the data disclosed herein demonstrate that RELMα is associated with glucose metabolism and the antidiabetic effects of TZDs. That is, the data disclosed herein demonstrate that RELMα expression is greatly and specifically increased upon exposure to TZDs, which are powerful antidiabetic drugs.

Moreover, RELMα is selectively expressed in the stromovascular cell compartment of white adipose tissue, further demonstrating that RELMα plays a role in glucose metabolism and diabetes, insulin resistance, Syndrome X, obesity, and the like. Additionally, the high degree of sequence homology between RELMα and resistin, which is known to play an important role in glucose uptake and obesity (see, e.g., WO 00/64920), further supports that RELMα plays a role in and/or is associated with insulin resistance.

The invention includes a method of diagnosing diabetes in an animal. The method comprises obtaining a biological sample from a first animal, assessing the level of resistin-like molecule α in the sample, and comparing that the level of resistin-like molecule α with the level of resistin-like molecule α in a biological sample obtained from a second otherwise identical animal not afflicted with diabetes. A higher level of resistin-like molecule α in the biological sample from the first animal compared with the level of resistin-like molecule α in the biological sample from the second otherwise identical animal is an indication that the first animal is afflicted with diabetes, thus diagnosing diabetes in an animal.

This is because, as stated previously elsewhere herein, the data disclosed herein demonstrate that RELMα is associated with glucose metabolism and the antidiabetic effects of TZDs. That is, the data disclosed herein demonstrate that RELMα expression is greatly and specifically increased upon exposure to TZDs, which are powerful antidiabetic drugs.

Moreover, RELMα is selectively expressed in the stromovascular cell compartment of white adipose tissue, further demonstrating that RELMα plays a role in glucose metabolism and diabetes, insulin resistance, Syndrome X, obesity, and the like. Additionally, the high degree of sequence homology between RELMα and resistin, which is known to play an important role in glucose uptake and obesity (see, e.g., WO 00/64920), further supports that RELMα plays a role in and/or is associated with diabetes.

The invention includes a method of assessing the effectiveness of a treatment for a colon tumor in a mammal. The method comprises assessing the level of RELMβ expression, amount, and/or activity, before, during and after a specified course of treatment for a disease, disorder or condition mediated by or associated with increased RELMβ expression (e.g., colon cancer, familial adenomatous polyposis, irritable bowel disease, inflammatory bowel disease, and intestinal tumors, among others). This is because, as stated previously elsewhere herein, RELMβ expression, amount and/or activity is associated with or mediates decreased increased cell proliferation which is feature of certain disease states. Thus, assessing the effect of a course of treatment upon RELMβ expression/amount/activity indicates the efficacy of the treatment such that a lower level of RELMβ expression, amount, or activity indicates that the treatment method is successful.

The invention further includes a method of assessing the effectiveness of a TZD therapy in an animal. The method comprises assessing the level of RELM (e.g., RELMα, RELMβ, or both) in an animal before, during, and/or after administration of TZD (e.g., rosiglitazone, troglitazone, pioglitazone, and the like). The level is compared among the various time points along the time course of TZD therapy and the skilled artisan would understand at what time point the level of RELM should be assessed, based on the course of TZD therapy, the condition for which the TZD is being administered, and other clinical and pharmacological parameters well known in the art which need not be repeated herein. A higher or lower level of RELM in the animal before TZD therapy compared with the level of TZD in the animal during and/or after therapy is an indication of the effectiveness of the TZD therapy in that animal. This is because, as more fully disclosed elsewhere herein, TZD affects the level of expression of RELMs (e.g., RELMα and RELMβ) in that administration of TZD increases the level of RELMα and RELMβ. Thus, the level of RELMα and/or RELMβ is an indication of the effectiveness of administration of TZD.

Alterations of RELMβ protein and/or RNA as likely to be useful in the diagnosis or prognosis of colon cancer or inflammatory bowel disease. Alterations of RELMα levels are likely to be of importance in the diagnosis and/or prognosis of insulin resistance and/or diabetes. For example, patients with high levels such as observed upon treatment with TZD are likely at less risk of insulin resistance and/or diabetes. RELMα levels are also likely to be useful in ascertaining favorable patient response to TZD.

The data disclosed herein should allow the identification and characterization of the RELM-receptor. This is useful since antagonism of the RELM receptor should provide useful in treatment of diseases, disorders or conditions mediated by RELM ligand/receptor signaling such as, but not limited to, colon cancer, familial adenomatous polyposis, irritable bowel disease, inflammatory bowel disease, intestinal tumors, breast cancer, tongue cancer, diabetes, insulin resistance, and obesity.

D. Methods of detecting fecal matter in a sample

The invention includes a novel method of detecting fecal matter in a sample. The method comprises detecting the presence or absence of RELMβ in the sample. This is because the data disclosed herein demonstrate that copious amounts of RELMβ are present and detectable in the stool. The data further demonstrate that RELMβ is excreted into the intestinal lumen since it is present in the mucous droplets in goblet cells. Thus, the skilled artisan would appreciate that the presence or absence of RELMβ in a sample is a marker for the presence or absence of fecal matter.

Detection of fecal matter in a sample has important implications in a number of settings including, but not limited to, assessment of water quality in water processing methods to provide safe, potable water, or safe swimming water, and also in settings where detection of fecal contamination is crucial, such as, but not limited to, food preparation procedures.

The skilled artisan would understand, based upon the disclosure provided herein, that the sample can include, but is not limited to, a food sample, a swimming water sample, a potable water sample, and any other material where detecting the presence or absence of fecal contamination is desired.

One skilled in the art would also appreciate, based upon the disclosure provided herein, that detecting the absence or presence of RELMβ in a sample encompasses methods of detecting the presence of a protein in a sample, which methods are well-known in the art or to be developed in the future. That is, the presence or absence of RELMβ in a sample can be assessed using an antibody that specifically binds with RELMβ, such immunoassays include, but are not limited to, radioirnmunoassays, ELISA-based assays, and the like.

Thus, the invention includes methods of assaying a sample for the presence or absence of RELMβ since the protein, which is produced in large amounts in the colon, is in indicator that there is fecal matter in the sample. Therefore, the invention provides a novel sensitive assay for detecting the presence of fecal contaminant in a sample.

The method comprises assessing the presence or absence of RELMβ in a sample, wherein the presence of RELMβ in the sample indicates that fecal matter is present in the sample. Further, the absence of RELMβ in the sample indicates the absence of fecal matter in that sample.

One skilled in the art would appreciate, based upon the disclosure provided herein, that a wide plethora of techniques are available for assessing the presence or absence of a protein, i.e., RELMβ, in a sample. These include, but are not limited to, immunoassays (e.g., ELISA), and western blot analysis, to detect RELMβ in a sample of interest. Further, the invention encompasses assaying many types of samples where the presence or absence of fecal matter is at issue. Such assays include, but are not limited to, assessing the presence of fecal matter in drinking water, swimming water, and in food preparation settings. One skilled in the art, once armed with the teachings of the invention, would understand that the methods disclosed herein can be used in a wide plethora of applications where detection of fecal matter is of interest.

IX. Kits

The invention includes various kits which comprise a compound, such as a nucleic acid encoding RELM, an antibody that specifically binds RELM, a nucleic acid complementary to a nucleic acid encoding RELM but in an antisense orientation with respect to transcription, and/or compositions of the invention, an applicator, and instructional materials which describe use of the compound to perform the methods of the invention. Although exemplary kits are described below, the contents of other useful kits will be apparent to the skilled artisan in light of the present disclosure. Each of these kits is included within the invention.

In one aspect, the invention includes a kit for alleviating a disease mediated by malexpression of RELM. The kit is used pursuant to the methods disclosed in the invention. Briefly, the kit may be used to contact a cell with a nucleic acid complementary to a nucleic acid encoding RELM where the nucleic acid is in an antisense orientation with respect to transcription to reduce expression of RELM, or with an antibody that specifically binds with RELM, wherein the decreased expression, amount, or activity of RELM mediates an beneficial effect. Moreover, the kit comprises an applicator and an instructional material for the use of the kit. These instructions simply embody the examples provided herein.

The kit includes a pharmaceutically-acceptable carrier. The composition is provided in an appropriate amount as set forth elsewhere herein. Further, the route of administration and the frequency of administration are as previously set forth elsewhere herein.

The skilled artisan would appreciate, based upon the disclosure provided herein, that the invention encompasses kits where ribozymes, antibodies that specifically bind with RELM, and the like, are comprised to reduce the level of RELM.

Further, the invention comprises kits comprising a nucleic acid encoding a mammalian RELM (e.g., RELMα, RELMβ, or both). Such kits can be used according to the methods of the invention wherever increased RELM is desired. That is, where a disease, disorder, or condition is associated with or mediated by decreased level of RELM compared with normal non-disease level of RELM, the kit can be used pursuant to the teachings disclosed elsewhere herein, to provide RELM to a cell wherein the level of RELM in the cell is less than the level of RELM in an otherwise identical but normal (i.e., not diseased) cell and/or to an animal comprising such a cell.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

### RELMβ and RELMα, novel resistin-like molecule family members

The experiments presented in this example may be summarized as follows.

The data disclosed herein demonstrate the discovery of a novel family of proteins, i.e., the family of resistin-like molecules (RELMs), in mammals (e.g., rodents and humans). Resistin, the first member of the family to be identified in mice and humans, is a novel hormone produced by fat cells. Resistin-like molecules alpha (RELMα) and beta (RELMβ), disclosed herein, are molecules that are highly related to resistin.

The data disclosed elsewhere herein demonstrate that RELMβ is expressed only in the gastrointestinal tract, particularly in the colon, in both mouse and human. RELMβ gene expression is highest in proliferative epithelial cells and is markedly increased in tumors, suggesting a role for RELMβ in intestinal cell proliferation. Both RELMβ and a third novel tissue-specific member of the family which is disclosed herein, termed RELMα, share a novel cysteine composition and other signature features with resistin. Both RELMβ and RELMα, like resistin, are secreted proteins. Unlike resistin, however, the expression of RELMβ and RELMα is significantly induced by the powerful antidiabetics, TZDs, such as rosiglitazone. Thus, the RELMs comprise a novel class of tissue-specific signaling molecules.

These data are discussed by Steppan et al. (2001, Proc. Natl. Acad. Sci. USA 98:502-506), which is incorporated by reference as if set forth in its entirety herein.

The Materials and Methods used in the experiments presented in this example are now described.

Cloning and isolation of mouse, human and rat RELM cDNAs

Cloning and isolation of RELM cDNAs was performed as described in International Application No. PCT/US00/11272 for resistin.

Northern and Western Blot Analyses

Northern and Western blot analyses were performed according to standard methods such as those described in, for example, Chawla et al. (1994, Endocrinology 135:798-800) and Huang et al. (2000, Genes Dev. 14:45-54), and International Application No. PCT/US00/11272.

Immunoblotting was performed using a mouse monoclonal FLAG antibody (Research Diagnostics, Inc., Flanders, N.J.) at a dilution of 1:2000.

Flag epitope tagged RELMβ and RELMα were produced according to standard methods, including, for example, standard methods described in Sambrook et al. (1989, Molecular Cloning: A Laboratory Manual, Cold Spring Harbor Laboratory, New York), Ausubel et al. (1997, Current Protocols in Molecular Biology, John Wiley & Sons, New York), and the like. Such methods further include those described in International Application No. PCT/US00/11272 for producing mammalian resistin tagged with a green fluorescent protein tag polypeptide.

In situ hybridization

In situ hybridization was performed essentially as described in Yang et al. (1998, Am. J. Physiol. 275:G1445-1453).

Sequence analysis

Sequences were analyzed using the NCBI Blast server available at the National Center for Biotechnology Information (NCBI) world wide web site having the universal resource locator (URL) “http://www.ncbi.nlm.nih.gov/BLAST/”.

Sequences were also analyzed using DNASTAR (DNASTAR, Inc., Madison, Wis.), Psort (publicly available at a wide web site having the URL “http://psort.nibb.ac.jp/”), and SignalP (publicly available at a wide web site having the URL “http://www.cbs.dtu.dk/services/SignalP-2.0/#submission/”) algorithms, all of which have been described previously.

The Results of the experiments presented in this example are now described.

Resistin is a newly described circulating protein with no significant sequence homology to any known hormone, cytokine, or other intercellular signaling molecule. Since many polypeptide signaling molecules are members of multigene families, a functional genomic strategy was applied in order to identify resistin homologs based upon the unique cysteine rich C-terminus of mouse and human resistin.

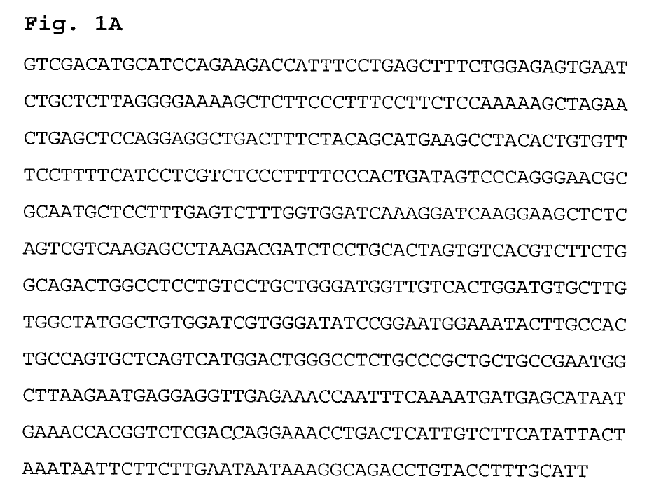

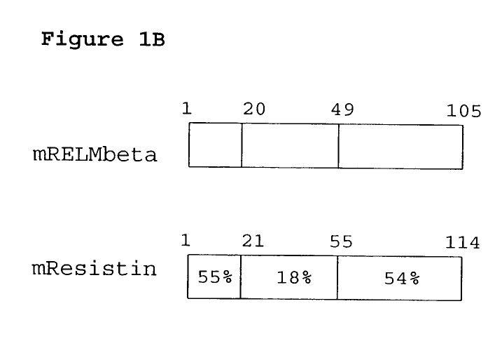

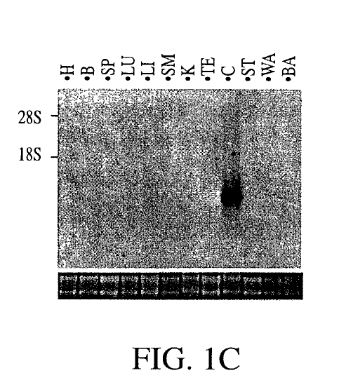

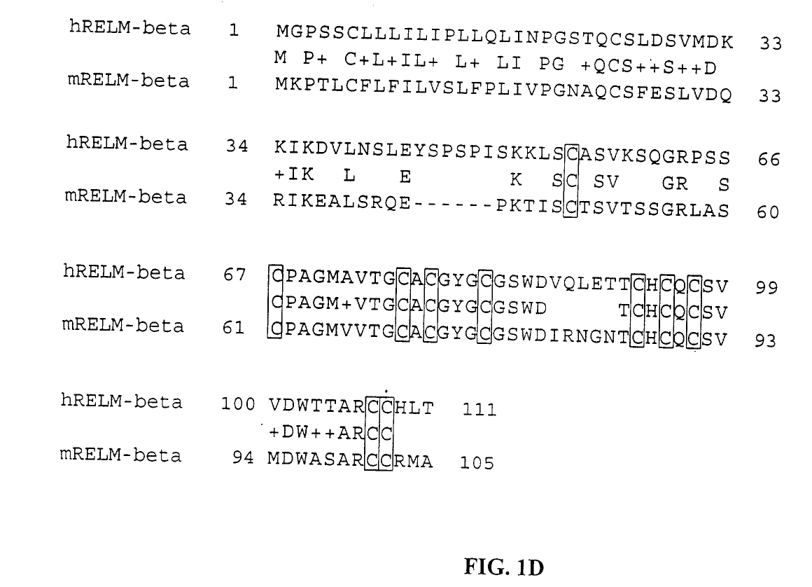

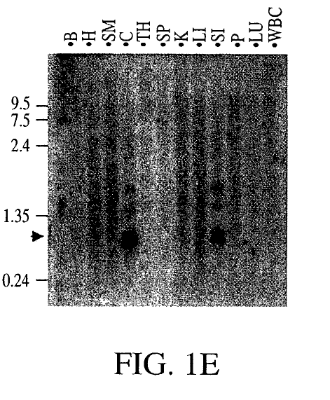

A search of the NCBI mouse EST database uncovered a series of expressed sequence tags (ESTs) encoding a novel resistin-like molecule (RELM), so termed because of its similarity to resistin, which is referred to as mouse RELMβ (mRELMβ). The mouse RELMβ cDNA (SEQ ID NO: 1) and deduced amino acid sequence (SEQ ID NO:2; (MKPTLCFLFILVSLFPLIVPGNAQCSFESLVDQRIKEALSRQEPKTISCTSVTSSG RLASCPAGMVVTGCACGYGCGSWDIRNGNTCHCQCSVMDWASARCCRMA) are depicted in FIG. 1A. RELMβ is highly related to resistin, especially in its conserved cysteine-containing C-terminus (FIG. 1B). The N-terminus of RELMβ is predicted by the PSORT and SignalP algorithms to contain a signal sequence that cleaves after the 20th amino acid. The predicted molecular weight of the processed form of RELMβ is 9003 daltons.

Like resistin, BLASTP search of the databases disclosed that the closest homologous protein, i.e., sharing the highest percent sequence identity with RELMs is ultra high-sulfur keratin 1 (GenBank Acc. No. S18946). However, even this protein 20 shares very little sequence identity with RELMs. For example, ultra high-sulfur keratin 1 shares only 32% identity over 21 of 65 amino acids with human RELMβ, but the full length human RELMβ comprises 111 amino acids. The BLASTP search results for human RELMβ demonstrating the limited sequence identity with human high sulfur keratin were as follows:

>pir||S37649 high-sulfur keratin—human

emb|CAA4493 8.1|(X6333 8) high sulfur keratin [Homo sapiens] Length=175

Score=32.8 bits (73), Expect=1.1

Identities=21/65 (32%), Positives=24/65 (36%), Gaps=17/65 (26%)

Query:63 RPSSCPAGMAVTGCACGYGCG------SW---DVQLETTCHCQCSVVDWTTARCCHL 110 +PS C TGC G G G W D ++E TC C VV T CC L

Sbjct:81 QPSCCQTSSCGTGCGIGGGIGYGQEGSSGAVSTRIRWCRPDCRVEGTCLPPCCVVSCHTPTCCQL 145

Thus, the searches of the relevant databases demonstrated that mammalian RELMs of the present invention are novel proteins.

To assess the potential biological role of RELMβ, Northern blot analysis was performed using RNA obtained from multiple mouse tissues in order to determine the tissue-specificity of RELM expression. Remarkably, RELMβ mRNA was abundant in colon but not in multiple other tissues including white adipose tissue, where resistin is uniquely expressed (FIG. 1C). Indeed, the vast majority of RELMβ ESTs isolated were derived from screening of colon libraries. Further, multiple overlapping ESTs were detected in and isolated from human colon libraries. The amino acid sequence of human RELMβ (SEQ ID NO:4; MGPSSCLLLILIPLLQLINPGSTQCSLDSVMDKKIKDVLNSLEYSPSPISKKLSCA SVKSQGRPSSCPAGMAVTGCACGYGCGSWDVQLETTCHCQCSVVDWTTARC CHLT) is depicted in FIG. 1D).

Human and mouse RELMβ are highly conserved, especially in the cysteine-rich C-terminus that is most similar to resistin (FIG. 1D). Importantly, the extreme N-terminus is predicted by the PSORT and SignalP algorithms to contain a signal sequence that cleaves after the 20th amino acid. Therefore, without wishing to be bound by any particular theory, RELMβ appears to be a secreted protein comprising a signal sequence which is cleaved from the mature form of the protein.

Northern analysis of multiple human tissues using the human RELMβ probe demonstrated that human RELMβ is also a small mRNA species detected only in colon and, to a lesser extent, in the small intestine (FIG. 1E).

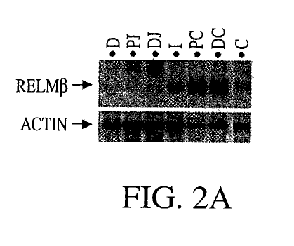

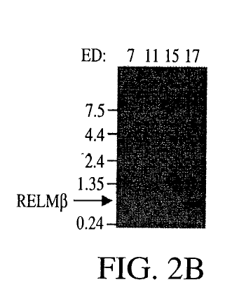

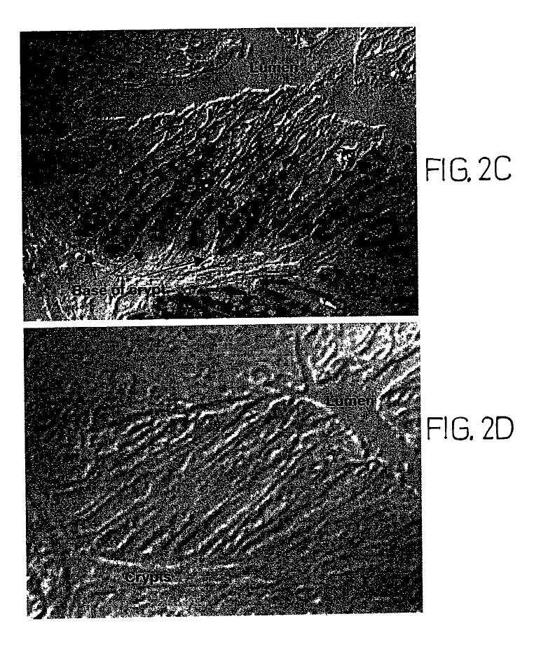

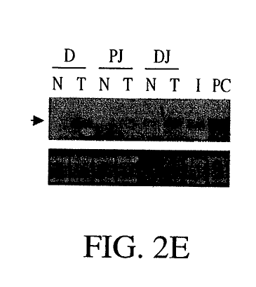

The data disclosed herein further demonstrate that, within the mouse intestine, RELMβ gene expression is by far most abundant in proximal and distal colon (FIG. 2A). RELMβ mRNA was also detected in the caecum, and the ileum, but at much lower levels than in the proximal and distal colon (FIG. 2A).

The data further demonstrate that during mouse embryogenesis, RELMβ gene expression is not detected until day 17, concurrent with acquisition of the adult colonic phenotype (FIG. 2B). Interestingly, this is the approximate stage of embryogenesis at which rodent colon undergoes significant remodeling with a transition from an undifferentiated stratified epithelium to a simple columnar epithelium (Williams & Bell, 1991, Embryol. 183:573-578).

Because the colon consists of multiple cell types, including epithelial cells, lamina propria cells, and the muscularis propria, in situ hybridization was performed to more precisely define where RELMβ is expressed in the colon. The data disclosed herein demonstrate that RELMβ mRNA is abundant in proliferative epithelia at the bases of the crypts and RELMβ RNA is dramatically extinguished in the non-proliferating differentiated epithelia, which have migrated up from the crypt base to the lumenal surface (FIG. 2C). The specificity of the in situ hybridization signal was confirmed by the lack of signal using a sense probe (FIG. 2D).

The robust expression of RELMβ in the proliferative compartment suggested that expression of RELMβ is associated with tumorgenesis. Thus, expression of RELMβ in min mice, which harbor a mutation of the APC gene and thus develop intestinal tumors similar to humans with familial adenomatous polyposis (Su et al., 1992, Science 256:668-670), was examined. As was observed in wild type mice, RELMβ expression was modest in normal duodenum and jejunum. However, in the min mouse, RELMβ mRNA was markedly increased in tumors (T) immediately adjacent to the normal (N) tissue (FIG. 2E). Thus, RELMβ expression is greatest in intestinal epithelial cells whose proliferative rate is increased due to normal as well as pathological mechanisms.

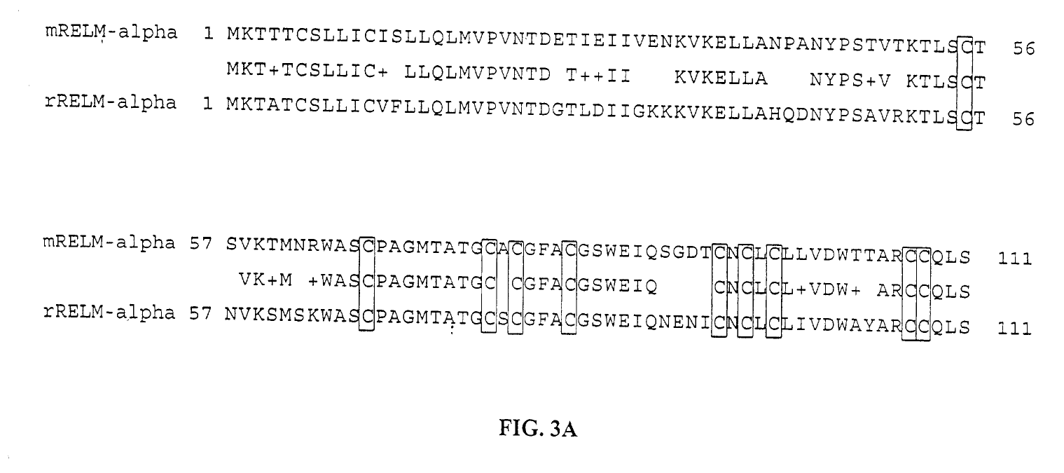

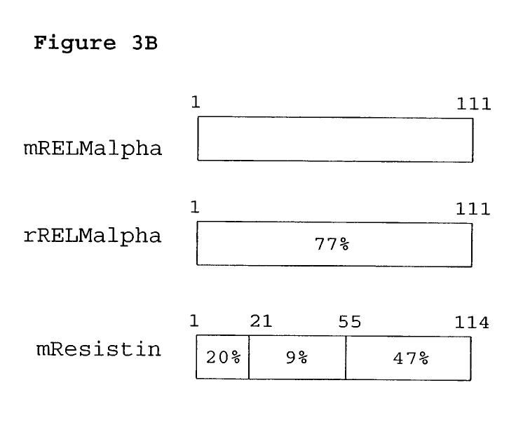

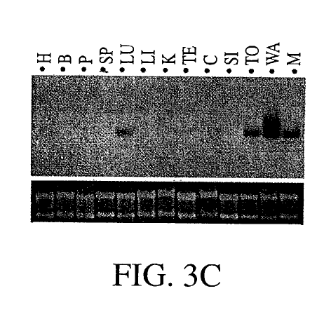

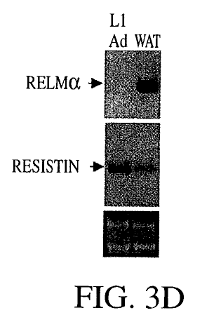

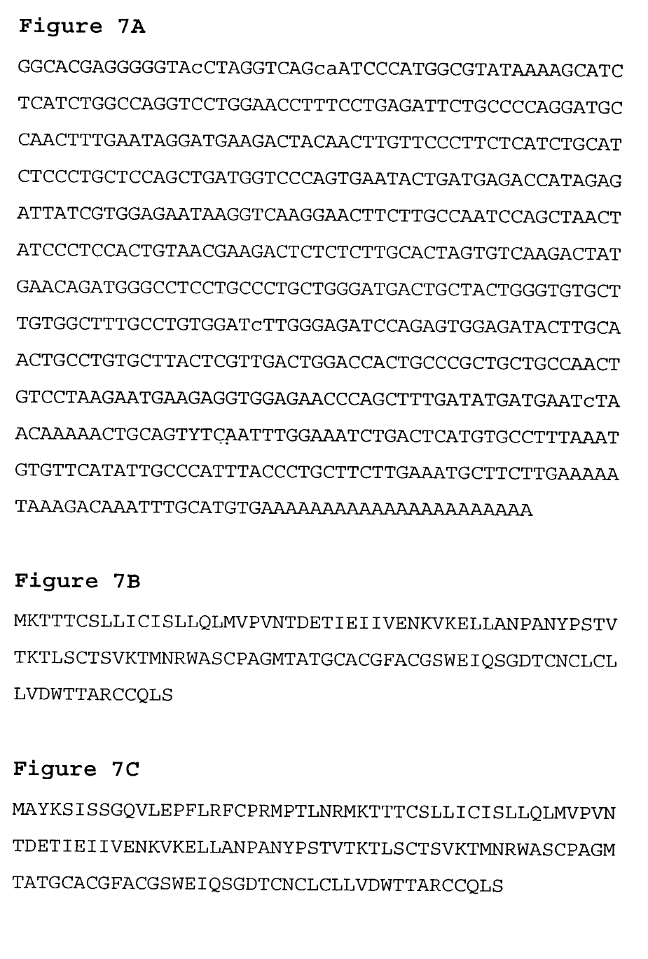

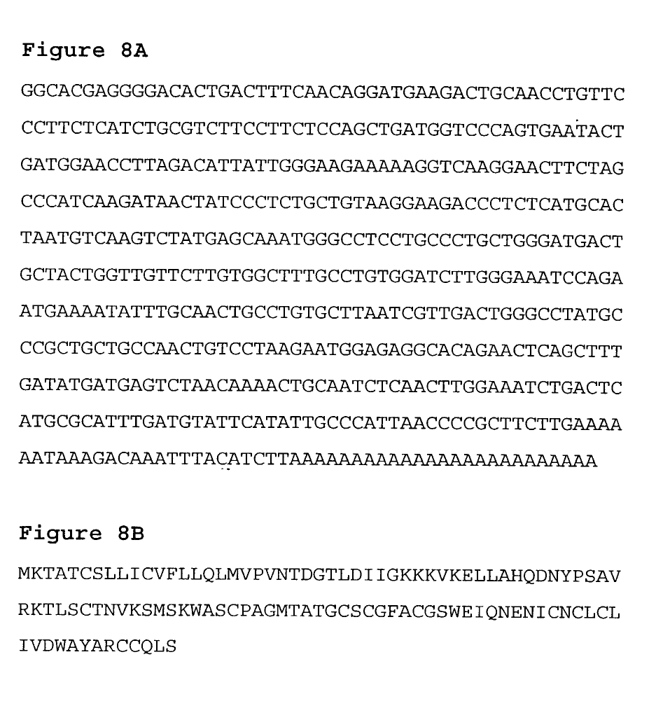

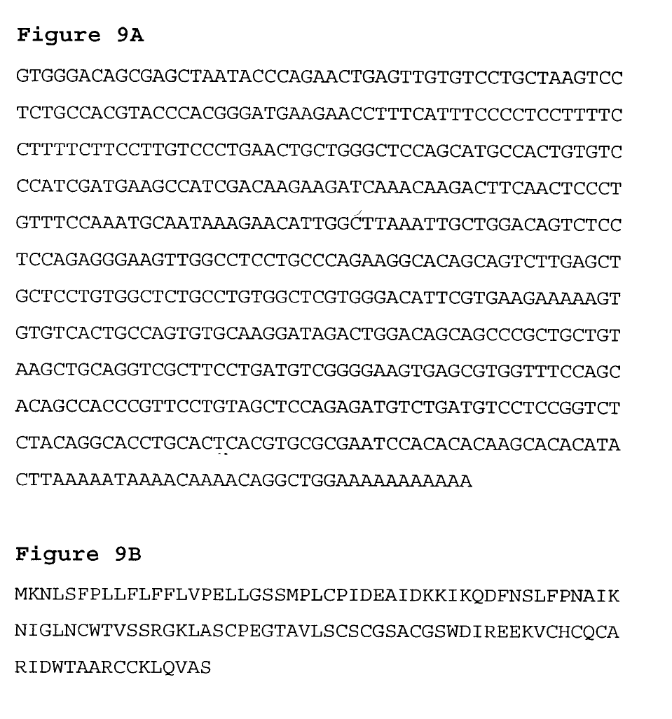

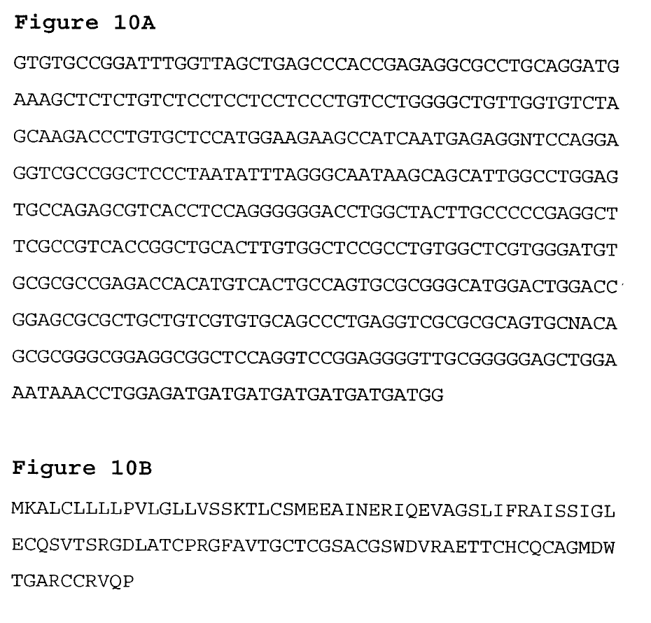

The data disclosed herein demonstrates the identification of a third member of the resistin family of protein, termed RELMβ, in mammary tissue, pancreas, and tongue mouse EST libraries (FIG. 3A). The N-terminus of mouse RELMα (mRELMα) contains a sequence that is predicted by the SignalP algorithm to be a signal sequence cleavable between amino acids 23 and 24. The amino acid sequence of mouse RELMα (SEQ ID NO:6; MKTTTCSLLICISLLQLMVPVNTDETIEIIVENKVKELLANPANYPSTVTKTLSC TSVKTMNRWASCPAGMTATGCACGFACGSWEIQSGDTCNCLCLLVDWTTAR CCQLS) is depicted in FIG. 3A. The amino acid sequence of an alternately translated mouse RELMα (SEQ ID NO:13; MAYKSISSGQVLEPFLRFCPRMPTLNRMKTTTCSLLICISLLQLMVPVNTDETIE IWENKVKELLANPANYPSTVTKTLSCTSVKTMNRWASCPAGMTATGCACGF ACGSWEIQSGDTCNCLCLLVDWTTARCCQLS) is depicted in FIG. 7C.

Rat RELMα (rRELMα) was identified as ESTs in lung and placental libraries. The amino acid sequence of rat RELMα (SEQ ID NO:8; MKTATCSLLICVFLLQLMVPVNTDGTLDIIGKKKVKELLAHQDNYPSAVRKTL SCTNVKSMSKWASCPAGMTATGCSCGFACGSWEIQNENICNCLCLIVDWAYA RCCQLS) is 77% identical to the predicted mouse protein (SEQ ID NO:6) (FIGS. 3A and 3B). Both RELMα proteins are only distantly related to resistin (SEQ ID NOs: 10 and 12) except in the C-terminal half that constitutes a “signature” sequence for the entire RELM family (FIG. 3B).

Northern blot data disclosed herein demonstrate that, like resistin, RELMα mRNA is most abundant in white adipose tissue (FIG. 3C). However, unlike resistin, RELMα it is not expressed in 3T3-L1 adipocytes (FIG. 3D) nor in preadipocytes, suggesting that, without wishing to be bound by any particular theory, RELMα may be produced by the stromal vascular constituents of adipose tissue. Also unlike resistin, RELMα is expressed is detected in tissue obtained from heart, lung, and tongue (FIG. 3D).

To determine whether RELMβ and RELMα are secreted proteins, their cDNAs, as well as that of resistin, were fused in frame to the flag epitope at the C-terminal end of the putative open reading frame of the RELM cDNA. The nucleic acid encoding the RELM-flag tag polypeptide fusion protein was transfected into 293T human embryonic kidney cells. Media was removed from the cultured transfectant cells and the proteins therein were assayed using Western blot using anti-flag antibody. The data disclosed herein demonstrate that, as disclosed by the analysis of the primary amino acid sequences disclosed previously elsewhere herein, RELMβ (β) and RELMα (α), like resistin, were secreted into the media by the transfected cells (FIG. 4A). These data suggest, without wishing to be bound by theory, that RELMβ and RELMα may be circulating hormones like resistin or they may function as paracrine, autocrine, or exocrine signaling molecules. Although various intestinal epithelial cell types secrete proteins with diverse functional roles (O'Neil et al., 1999, J. Immunol. 163:6718-6724; Barnard et al., 1995, Gastroenterology 108:564-580; Roth et al., 1992, Am. J. Physiol. 263:G174-180; Sands & Podolsky, 1996, Ann. Rev. Physiol. 58:253-273), RELMβ is unique both in its structure and in its pattern of expression.

The consensus RELM is thus a protein of about 105-114 amino acid in lengths comprising three domains: an N-terminal signal sequence, a variable middle portion, and a highly conserved C-terminal “signature” sequence that comprises nearly half of the molecule (FIG. 4B). The signature region of the RELMs contains a unique and invariant spacing of the cysteine residues: C-X₁₁-C-X₈-C-X-C-X₃-C-X₁₀-C-X-C-X-C-X₉-CC-X₃₋₆-END. This cysteine residue array is reminiscent of, but clearly distinct from, so-called “EGF repeats” (epidermal growth factor) that are characteristic of a number of signaling molecules (Campbell & Bork, 1993, Curr. Biol. 3:385-392).

Without wishing to be bound by any particular theory, the cysteine pattern of the RELMs contributes to protein folding and, potentially, to multimerization of the proteins. Further, without wishing to be bound by any particular theory, the highly conserved C-terminus, as well as the cysteine conservation, may contribute to binding to a related family of receptors that are yet to be discovered.

To date, RELMs have not been identified in the nearly completed sequenced genomes of Caenorhabditis elegans or Drosophila melanogaster, suggesting, without wishing to be bound by any particular theory, that RELMs may be specific to higher organisms. Nevertheless, it is likely that additional RELMs will be discovered as the sequencing of the human and other genomes is completed. Without wishing to be bound by any particular theory, the data disclosed herein suggests that each RELM has distinct biological functions consistent with its unique pattern of expression.

RELMα expression is limited to mammary tissue, tongue, and white adipose tissue (i.e., fat). Diseases of these tissues include carcinoma of the breast and mouth. Excess white adipose tissue is the sine qua non of obesity, which is epidemic in industrialized societies and has tremendous associated morbidity including cardiovascular disease, diabetes, and cancer (Medical Clinics of North America, entire volume, March 2000).

Thus, the data disclosed herein demonstrate that RELM is a potential diagnostic tool for colon cancer, familial adenomatous polyposis, irritable bowel disease, inflammatory bowel disease, intestinal tumors, breast cancer, and tongue cancer, which diseases are characterized by cell proliferation and increased expression of RELM. Further, the data disclosed herein suggest that certain RELMs are diagnostics and therapeutics with regard to diseases, disorders or conditions such as, but not limited to, diabetes, insulin resistance, obesity, Syndrome X, polycystic ovary disease, and other diseases, disorders or conditions associated with glucose metabolism. The data disclosed herein also indicate that RELM is a potential target for novel therapies for colon cancer, familial adenomatous polyposis, irritable bowel disease, inflammatory bowel disease, intestinal tumors, breast cancer, tongue cancer, diabetes, insulin resistance, and obesity, which therapies function by decreasing RELM levels, decreasing RELM biological activity, or both, or therapies based on antagonism of the cellular receptor for RELM thereby inhibiting RELM/receptor interactions involved in colon cancer, familial adenomatous polyposis, irritable bowel disease, inflammatory bowel disease, intestinal tumors, breast cancer, tongue cancer, diabetes, insulin resistance, and obesity, and other diseases, disorders, or conditions associated with or mediated by increased expression of RELM.

### EXAMPLE 2

### RELMα

The experiments presented in this example may be summarized as follows.

The data disclosed herein further demonstrate the specific expression of RELMα in white adipose tissue. Moreover, the data indicate that RELMα is expressed in non-adipocytes in that tissue, i.e., it is expressed in stromal-vascular (SV) cells in white adipose tissue. The data disclosed herein further demonstrate that RELMα expression is greatly increased by the antidiabetic drug, rosiglitazone.

The Materials and Methods used in the experiments presented in this example are essentially the same as those described in Example 1.

As demonstrated previously elsewhere herein, RELMα expression is most abundant in white adipose tissue. However, RELMα is not expressed in 3T3-L1 mouse adipocytes in culture, suggesting that it is not expressed in adipocytes but rather in non-adipocyte cells, which are collectively referred to as stromal vascular (SV) cells, in fat tissue.

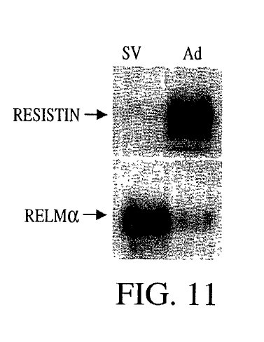

To determine whether RELMα is selectively expressed in SV cells and not in adipocytes, mouse white adipose tissue was fractionated into adipocytes and SV cells using standard techniques. Northern blot analysis was performed in that twenty micrograms of total RNA from adipocytes (Ad) or SV cells were loaded into each gel lane and the blots were hybridized with cDNA probes for resistin and RELMα. Resistin mRNA, which was previously demonstrated to expressed in adipocytes, was detected almost exclusively in the adipocyte fraction (FIG. 11). In contrast, the data disclosed herein demonstrate that RELMα expression was detected almost exclusively in the SV compartment of white adipose tissue. Without wishing to be bound by any particular theory, the small amounts of resistin in SV RNA and of RELMα in AdRNA most likely represent minor contamination related to the method of enrichment of the two fractions.

The stromal vascular cells function as a depot for preadipocytes, as well as providing oxygen and other nutrients to adipocytes. The data disclosed herein suggest that RELMα provides a means of communication from the stromal vascular compartment to the adipocytes. Thus, the data disclosed herein suggest that RELMα plays a role in regulating the expression of resistin, other key genes, or both, or in regulation of fat storage and metabolism in adipocytes.

To determine whether RELMα is regulated by antidiabetic drugs, the level of RELMα expression in response to rosiglitazone was evaluated in the art-recognized ob/ob mouse model of obesity and type 2 diabetes. Rosiglitazone is a thiazolidinedione (TZD), which is a new class of orally active drugs that are particularly exciting because they decrease insulin resistance by enhancing the actions of insulin at a level distal to the insulin receptor (Henry, 1997, Endo. Metab. Clin. North Amer. 26:553-573). TZDs, which include troglitazone, pioglitazone, and rosiglitazone are thought to sensitize target tissues to the action of insulin.

TZDs were originally developed by screening analogs of clofibric acid for anti-pipidemic and anti-hyperglycemic effects (Kawamatsu et al., 1980, Arznein.-Forsch./Drug Res. 30:454-459). The anti-diabetic effects of these compounds were not understood but the discovery that TZDs enhanced adipocyte differentiation (Hiragun et al., 1988, J. Cell. Physiol. 134:124-130; Kletzien et al., 1992, Mol. Pharmacol. 41:393-398) was an important clue to identifying its molecular target. Activators of peroxisome proliferator activated receptor γ (PPARγ), a member of the nuclear hormone receptor superfamily, were also found to induce adipogenesis. PPARγ was shown to be predominantly expressed in adipose tissue and to function as a key transcription factor in adipocyte differentiation (Tontonoz et al., 1994, Cell 79:1147-1156). Shortly after these studies, TZDs were demonstrated to be direct ligands for PPARγ.

PPARγ belongs to a subset of nuclear receptors that forms heterodimers with the retinoid X receptor (RXR), which greatly enhances the ability of the receptor to bind specific DNA sequences in target genes. The DNA sequences recognized by the PPAR/RXR heterodimer are referred to as PPAR-response elements (PPREs). PPAR/RXR heterodimers bind to PPREs in the absence of ligand, but binding of the ligand leads to a conformational change which results in activation of transcription of the target gene. The active conformation recruits a multiprotein coactivator complex that acetylates histones (leading to an open, more active conformation of the nucleosome) as well as interacting directly with the basal transcription machinery. PPREs have been found in the regulatory regions of a number of genes involved in lipid metabolism and energy balance (Lemberger et al., 1996, Annu. Rev. Cell Dev. Biol. 12:335-363).

There is strong evidence that TZDs function via PPARγ. PPARγ has been shown to bind to a number of different ligands including a number of fatty acids as well as prostaglandin J derivatives, such as 15-deoxy-Δ12,14-prostaglandin J2 and others (Forman et al., 1995, Cell 81:541-550; Kliewer et al., 1997, Proc. Natl. Acad. Sci. USA 94:4318-4323). However, none of these compounds binds to PPARγ with affinities in the nanomolar range. In contrast, TZDs have been shown to bind to PPARγ with an affinity in the range of 40-200 nM. Not only are TZDs activating ligands for PPARγ at nanomolar concentrations, there is also a remarkable correlation between TZD potencies for in vivo plasma glucose lowering with their order of potency for both PPARγ activation and direct binding to PPARγ (Wilson, 1996, J. Med. Chem. 39:665-668; Berger et al., 1996, Endocrinology 137:4189-4195). RXR ligands can also activate the PPARγ/RXR heterodimer, and synthetic RXR agonists increase insulin sensitivity in obese mice and work in combination with TZDs to further enhance antidiabetic activity (Mukherjee, et al., 1997, Nature 386:407-410). This further suggests that the PPARγ/RXR heterodimer complex is the molecular target for treatment of insulin resistance in vivo.

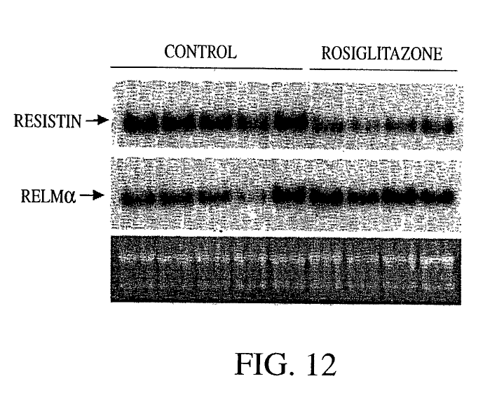

Twenty micrograms of total RNA from control vehicle- or rosiglitazone-treated mice was assayed using Northern blot analysis using cDNA probes for resistin and RELMα (FIG. 12). The data disclosed herein demonstrate that RELMα gene expression was markedly increased by rosiglitazone treatment, suggesting that RELMα may play a role in the glucose lowering actions of rosiglitazone and related antidiabetic thiazolidinediones and other antidiabetic drugs that target the nuclear receptor PPARγ. Without wishing to be bound by any particular theory, secreted RELMα may act as a secreted protein upon muscle and other insulin responsive tissues, in addition to acting more locally on adipocytes. The induction of RELMα by rosiglitazone is in striking contrast to the down regulation of resistin by the same treatment in the same mice. Without wishing to be bound by any particular theory, these data suggest that the effects of RELMα and resistin are antagonistic, and that by increasing RELMα while decreasing resistin expression the ratio of RELMα to resistin is dramatically increased upon exposure to TZDs.

### EXAMPLE 3

### RELMβ

The experiments presented in this example may be summarized as follows.

The data disclosed herein demonstrate the specific expression of RELMβ in mouse and human stool, mouse colon, and in human intestinal cell line. Moreover, the data demonstrate that the level of RELMβ RNA expression and the level of RELMβ protein is greatly decreased in the colon of mice raised under germ-free conditions compared with the level of RELMβ RNA expression and the level of RELMβ protein in the colon of mice raised under normal conditions. Further, the data disclosed herein demonstrate that the level of RELMβ RNA expression is markedly increased by the administration of the TZD, rosiglitazone, in a dose-dependent manner.

The Materials and Methods used in the experiments presented in this example are essentially the same as those described in Example 1.

As disclosed previously elsewhere herein, RELMβ expression is dramatically restricted to intestinal tissue, and it is especially predominant in the colon. The data disclosed previously elsewhere herein demonstrate that RELMβ mRNA was expressed in colonic epithelia.

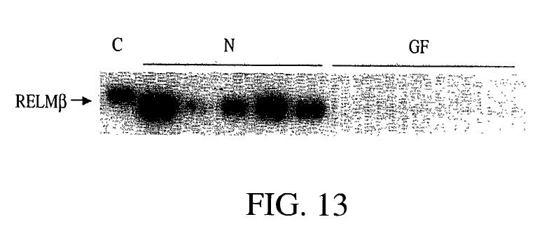

The data disclosed herein demonstrate that in structural studies using RELMβ-specific antibody, the protein is localized to mucous droplets in goblet cells. These cells secrete their contents into the intestinal lumen. Western blot analysis of RELMβ protein expression demonstrates that normal mouse stool contains copious amounts of RELMβ protein (FIG. 13). This protein is expected to act from the luminal side, potentially acting on luminal contents which include bacteria. Bacteria in the gut are known to play a causative role in inflammatory bowel disease.

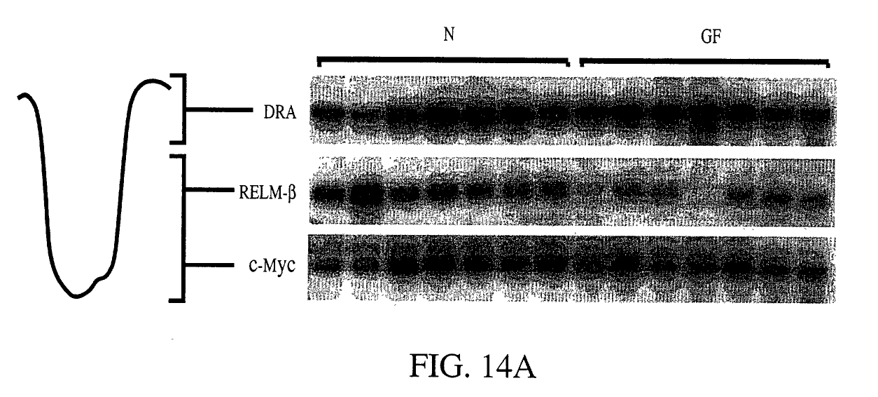

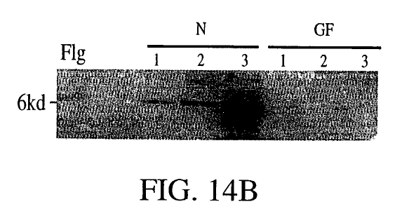

To examine the potential role of bacteria in RELMβ expression, mice were reared in a germ free environment. These mice do not have bacteria in their colon. Remarkably, they also do not have RELMβ in their stool (FIG. 13, right lanes). Thus RELMβ is likely to have an important effect in stool, and this effect is likely to relate to bacteria and their consequences, including bacterial-associated inflammatory bowel disease. RELMβ RNA expression (FIG. 14A) and protein level (FIG. 14B) in the colon were similarly reduced in the germ-free environment compared with normal conditions. Together, these data suggest that RELMβ plays an important role in the intestinal response to bacteria and is involved in and/or associated with inflammatory bowel disease.

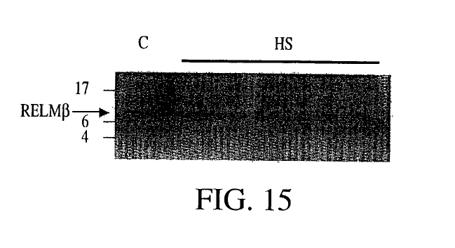

The data disclosed previously elsewhere herein demonstrate that RELMβ mRNA is expressed in human colon, and Western blot analysis of normal human stool further demonstrated that RELMβ protein is found in human stool (FIG. 15).

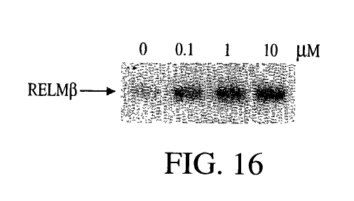

Colon cells also express abundant PPARγ. Since data disclosed previously elsewhere herein demonstrate that related molecules resistin and RELMβ are regulated by thiazolidinediones, and TZDs have been shown to improve inflammatory bowel disease, the effect of rosiglitazone on RELMβ gene expression in a human intestinal cell line (LS124T cells) was assessed. Northern blot analysis indicated that rosiglitazone treatment markedly increased RELMβ gene expression in a dose-dependent manner (FIG. 16).

Without wishing to be bound by any particular theory, the ability of gut comensal bacteria to induce the expression of RELMβ by the colonic epithelium leading to its secretion into the stool, where it exists at high concentration without evidence of proteolysis, suggests that RELMβ has an influence on gut bacteria. Epithelial cells in plants, invertebrates, and vertebrates (including humans) produce various peptide antibiotics that prevent bacteria from entering mucosal surfaces (Schroder, 1999, CMLS 56:32-46). Specialized epithelial cells located in the base on small intestinal crypts known as Paneth cells secrete small, cysteine-rich peptides with potent anti-microbial activity known as defensins (Bevins et al., 1999, Gut 45:911-915). Although the structure of RELMβ is distinct from that of defensins, defensin and resistin-like molecules have several similarities: 1) both are relatively small peptides, 2) both proteins are cysteine-rich, and 3) both proteins are secreted apically by epithelial cells into the intestinal lumen.

Recently, antimicrobial peptides from animals and plants have served as templates for the design of new therapeutic antibiotics (Cole et al., 2000, BioTechniques 29:822-831). Therefore, without wishing to be bound by any particular theory, the data disclosed herein indicate that RELMβ, similarly to mammalian defensins, has antimicrobial activities and can serve as a template for the development of novel antimicrobial agents. Further, the high degree of sequence homology shared by RELMα and RELMβ with resistin, which is known to be involved in glucose metabolism (see, e.g., International Publication No. WO 00/64920), indicates that these molecules are also involved in and/or play a role in glucose metabolism and diseases, disorders or conditions associated with glucose metabolism, such as, but not limited to, insulin resistance, Syndrome X, diabetes, and obesity.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated nucleic acid encoding a mammalian resistin-like molecule, or a fragment thereof.

2. The isolated nucleic acid of claim 1, wherein said isolated nucleic acid shares at least about 30% sequence identity with a nucleic acid encoding at least one of a mouse resistin-like molecule β (SEQ ID NO:1), a human resistin-like molecule β (SEQ ID NO:3), a mouse resistin-like molecule α (SEQ ID NO:5), and a rat resistin-like molecule α (SEQ ID NO:7).

3. An isolated nucleic acid encoding a mammalian resistin-like molecule, wherein the amino acid sequence of said resistin-like molecule shares at least about 30% sequence identity with an amino acid sequence of at least one of SEQ ID NO:2, SEQ ID NO:4, SEQ ID NO:6, SEQ ID NO:8, and SEQ ID NO:13.

4. An isolated polypeptide comprising a mammalian resistin-like molecule.

5. An isolated polypeptide comprising a mammalian resistin-like molecule, wherein said mammalian resistin-like molecule shares at least about 30% sequence identity with at least one amino acid sequence selected from the group consisting of SEQ ID NO:2, SEQ ID NO:4, SEQ ID NO:6, SEQ ID NO:8, and SEQ ID NO:13.

6. The isolated nucleic acid of claim 1, said nucleic acid further comprising a nucleic acid encoding a tag polypeptide covalently linked thereto.

7. The isolated nucleic acid of claim 6, wherein said tag polypeptide is selected from the group consisting of a myc tag polypeptide, a glutathione-S-transferase tag polypeptide, a green fluorescent protein tag polypeptide, a myc-pyruvate kinase tag polypeptide, a His6 tag polypeptide, an influenza virus hemagglutinin tag polypeptide, a flag tag polypeptide, and a maltose binding protein tag polypeptide.

8. The isolated nucleic acid of claim 1, said nucleic acid further comprising a nucleic acid specifying a promoter/regulatory sequence operably linked thereto.

9. A vector comprising the isolated nucleic acid of claim 1.

10. The vector of claim 9, said vector further comprising a nucleic acid specifying a promoter/regulatory sequence operably linked thereto.

11. A recombinant cell comprising the isolated nucleic acid of claim 1.

12. A recombinant cell comprising the vector of claim 9.

13. An isolated nucleic acid complementary to an isolated nucleic acid encoding a mammalian resistin-like molecule, or a fragment thereof, said complementary nucleic acid being in an antisense orientation.

14. The isolated nucleic acid of claim 13, wherein said nucleic acid shares at least about 30% identity with a nucleic acid complementary with a nucleic acid having the sequence of at least one of a mouse resistin-like molecule β (SEQ ID NO: 1), a human resistin-like molecule β (SEQ ID NO:3), a mouse resistin-like molecule α (SEQ ID NO:5), and a rat resistin-like molecule α (SEQ ID NO:7).

15. A recombinant cell comprising the isolated nucleic acid of claim 13.

16. A vector comprising the isolated nucleic acid of claim 13.

17. An antibody that specifically binds with a mammalian resistin-like molecule polypeptide, or a fragment thereof.

18. An antibody that specifically binds with a mammalian resistin-like molecule polypeptide, or a fragment thereof, wherein said mammalian resistin-like molecule shares at least about 30% sequence identity with at least one amino acid sequence selected from the group consisting of SEQ ID NO:2, SEQ ID NO:4, SEQ ID NO:6, SEQ ID NO:8, and SEQ ID NO:13.

19. The antibody of claim 18, wherein said antibody is selected from the group consisting of a polyclonal antibody, a monoclonal antibody, a humanized antibody, a chimeric antibody, and a synthetic antibody.

20. A composition comprising an antibody that specifically binds with a mammalian resistin-like molecule polypeptide, or a fragment thereof, and a pharmaceutically-acceptable carrier.

21. The composition of claim 19, wherein said mammalian resistin-like molecule polypeptide shares at least about 30% sequence identity with at least one amino acid sequence selected from the group consisting of SEQ ID NO:2, SEQ ID NO:4, SEQ ID NO:6, SEQ ID NO:8, and SEQ ID NO:13, or a fragment thereof.

22. A composition comprising an isolated nucleic acid complementary to an isolated nucleic acid encoding a mammalian resistin-like molecule, or a fragment thereof, said complementary nucleic acid being in an antisense orientation, and a pharmaceutically-acceptable carrier.

23. The composition of claim 22, wherein said isolated nucleic acid encoding a mammalian resistin-like molecule, or a fragment thereof, shares at least about 30% sequence identity with a nucleic acid encoding at least one of a mouse resistin-like molecule β (SEQ ID NO:1), a human resistin-like molecule β (SEQ ID NO:3), a mouse resistin-like molecule α (SEQ ID NO:5), and a rat resistin-like molecule α (SEQ ID NO:7).

24. A composition comprising an isolated nucleic acid encoding a mammalian resistin-like molecule, or a fragment thereof, and a pharmaceutically-acceptable carrier.

25. The composition of claim 24, wherein said isolated nucleic acid encoding a mammalian resistin-like molecule, or a fragment thereof, shares at least about 30% sequence identity with a nucleic acid encoding at least one of a mouse resistin-like molecule β (SEQ ID NO: 1), a human resistin-like molecule β (SEQ ID NO:3), a mouse resistin-like molecule α (SEQ ID NO:5), and a rat resistin-like molecule α (SEQ ID NO:7).

26. A composition comprising an isolated polypeptide comprising a mammalian resistin-like molecule, and a pharmaceutically-acceptable carrier.

27. The composition of claim 26, wherein said mammalian resistin-like molecule shares at least about 30% sequence identity with at least one amino acid sequence selected from the group consisting of SEQ ID NO:2, SEQ ID NO:4, SEQ ID NO:6, SEQ ID NO:8, and SEQ ID NO:13.

28. A transgenic non-human mammal comprising an isolated nucleic acid encoding a mammalian resistin-like molecule, or a fragment thereof.

29. The transgenic non-human mammal of claim 28, wherein said isolated nucleic acid shares at least about 30% sequence identity with a nucleic acid encoding at least one of a mouse resistin-like molecule β (SEQ ID NO: 1), a human resistin-like molecule β (SEQ ID NO:3), a mouse resistin-like molecule α (SEQ ID NO:5), and a rat resistin-like molecule α (SEQ ID NO:7).

30. A method of treating a disease mediated by malexpression of a resistin-like molecule alpha in a human, said method comprising administering to a human patient afflicted with a disease mediated by malexpression of a resistin-like molecule α, a resistin-like molecule α expression-inhibiting amount of a composition comprising an isolated nucleic acid complementary to an isolated nucleic acid encoding a resistin-like molecule α, or a fragment thereof, said complementary nucleic acid being in an antisense orientation, said composition further comprising a pharmaceutically-acceptable carrier.

31. The method of claim 30, wherein said isolated nucleic acid encoding resistin-like molecule a shares at least about 30% sequence identity with at least one of a nucleic acid encoding a mouse resistin-like molecule β (SEQ ID NO: 1), a human resistin-like molecule β (SEQ ID NO:3), a mouse resistin-like molecule α (SEQ ID NO:5), and a rat resistin-like molecule α (SEQ ID NO:7).

32. The method of claim 30, wherein said disease is selected from the group consisting of breast cancer, tongue cancer, insulin resistance, diabetes, Syndrome X, and obesity.

33. A method of treating a disease mediated by malexpression of a resistin-like molecule β in a human, said method comprising administering to a human patient afflicted with a disease mediated by malexpression of a resistin-like molecule βI, a resistin-like molecule β expression-inhibiting amount of a composition comprising an isolated nucleic acid complementary to an isolated nucleic acid encoding a resistin-like molecule β, or a fragment thereof, said complementary nucleic acid being in an antisense orientation, said composition further comprising a pharmaceutically-acceptable carrier.

34. The method of claim 33, wherein said isolated nucleic acid encoding resistin-like molecule β shares at least about 30% sequence identity with at least one of a nucleic acid encoding a mouse resistin-like molecule β (SEQ ID NO: 1), a human resistin-like molecule β (SEQ ID NO:3), a mouse resistin-like molecule α (SEQ ID NO:5), and a rat resistin-like molecule α (SEQ ID NO:7).

35. The method of claim 34, wherein said disease is selected from the group consisting of irritable bowel disease, inflammatory bowel disease, colon cancer, familial adenomatous polyposis, and an intestinal tumor.

36. A method of diagnosing a colon tumor in an animal, said method comprising obtaining a biological sample from a first animal, assessing the level of resistin-like molecule β in said biological sample, and comparing the level of resistin-like molecule β in said biological sample with the level of resistin-like molecule β in a biological sample obtained from a second otherwise identical animal, wherein a higher level of resistin-like molecule β in said biological sample from said first animal compared with the level of resistin-like molecule β in said biological sample from said second otherwise identical animal is an indication that first said animal is afflicted with a colon tumor, thereby diagnosing a colon tumor in an animal.

37. The method of claim 36, wherein said biological sample is selected from the group consisting of a blood sample, a lung biopsy sample, a fat biopsy sample, a stool sample, and a cerebrospinal fluid sample.

38. A method of diagnosing familial adenomatous polyposis in an animal, said method comprising obtaining a biological sample from a first animal, assessing the level of resistin-like molecule β in said biological sample, and comparing the level of resistin-like molecule β in said biological sample with the level of resistin-like molecule β in a biological sample obtained from a second otherwise identical animal not afflicted with inflammatory bowel disease, wherein a higher level of resistin-like molecule β in said biological sample from said first animal compared with the level of resistin-like molecule β in said biological sample from said second otherwise identical animal is an indication that said first animal is afflicted with familial adenomatous polyposis, thereby diagnosing familial adenomatous polyposis in an animal.

39. A method of diagnosing inflammatory bowel disease in an animal, said method comprising obtaining a biological sample from a first animal, assessing the level of resistin-like molecule β in said biological sample, and comparing the level of resistin-like molecule β in said biological sample with the level of resistin-like molecule β in a biological sample obtained from a second otherwise identical animal not afflicted with inflammatory bowel disease, wherein a higher level of resistin-like molecule β in said biological sample from said first animal compared with the level of resistin-like molecule β in said biological sample from said second otherwise identical animal is an indication that said first animal is afflicted with inflammatory bowel disease, thereby diagnosing inflammatory bowel disease in an animal.

40. A method of diagnosing insulin resistance in an animal, said method comprising obtaining a biological sample from a first animal, assessing the level of resistin-like molecule α in said biological sample, and comparing the level of resistin-like molecule α in said biological sample with the level of resistin-like molecule α in a biological sample obtained from a second otherwise identical animal not afflicted with insulin resistance, wherein a higher level of resistin-like molecule α in said biological sample from said first animal compared with the level of resistin-like molecule α in said biological sample from said second otherwise identical animal is an indication that said first animal is afflicted with insulin resistance, thereby diagnosing insulin resistance in an animal.

41. A method of diagnosing diabetes in an animal, said method comprising obtaining a biological sample from a first animal, assessing the level of resistin-like molecule α in said biological sample, and comparing the level of resistin-like molecule α in said biological sample with the level of resistin-like molecule α in a biological sample obtained from a second otherwise identical animal not afflicted with diabetes, wherein a higher level of resistin-like molecule α in said biological sample from said first animal compared with the level of resistin-like molecule α in said biological sample from said second otherwise identical animal is an indication that said first animal is afflicted with diabetes, thereby diagnosing diabetes in an animal.

42. A method of assessing the effectiveness of thiazolidinedione (TZD) therapy in an animal, said method comprising assessing a level of a resistin-like molecule in said animal before, during or after administration of a TZD to said animal, wherein a higher or lower level of said resistin-like molecule in said animal during or after administration of said TZD compared with said level of said resistin-like molecule in said animal before administration of said TZD is an indication of the effectiveness of said TZD therapy in said animal, thereby assessing the effectiveness of TZD therapy in said animal.

43. The method of claim 42, wherein said thiazolidinedione is selected from the group consisting of a rosiglitazone, a troglitazone, and a pioglitazone.

44. A method of identifying a compound that affects expression of resistin-like molecule in a cell, said method comprising contacting a cell with a test compound and comparing the level of resistin-like molecule expression in said cell with the level of resistin-like molecule expression in an otherwise identical cell not contacted with said test compound, wherein a higher or lower level of resistin-like molecule expression in said cell contacted with said test compound compared with the level of resistin-like molecule expression in said otherwise identical cell not contacted with said test compound is an indication that said test compound affects expression of resistin-like molecule in a cell, thereby identifying a compound that affects expression of resistin-like molecule in a cell.

45. A compound identified by the method of claim 44.

46. The method of claim 44, wherein said resistin-like molecule is selected from the group consisting of a resistin-like molecule α and a resistin-like molecule β.

47. A method of identifying a compound that reduces expression of a resistin-like molecule in a cell, said method comprising contacting a cell with a test compound and comparing the level of resistin-like molecule expression in said cell with the level of resistin-like molecule expression in an otherwise identical cell not contacted with said test compound, wherein a lower level of resistin-like molecule expression in said cell contacted with said test compound compared with the level of resistin-like molecule expression in said otherwise identical cell not contacted with said test compound is an indication that said test compound reduces expression of resistin-like molecule in said cell, thereby identifying a compound that reduces expression of resistin-like molecule in a cell.

48. A compound identified by the method of claim 47.

49. A method of identifying a compound that increases expression of resistin-like molecule in a cell, said method comprising contacting a cell with a test compound and comparing the level of resistin-like molecule expression in said cell with the level of resistin-like molecule expression in an otherwise identical cell not contacted with said test compound, wherein a higher level of resistin-like molecule expression in said cell contacted with said test compound compared with the level of resistin-like molecule expression in said otherwise identical cell not contacted with said test compound is an indication that said test compound increases expression of resistin-like molecule in a cell, thereby identifying a compound that increases expression of resistin-like molecule in a cell.

50. A compound identified by the method of claim 49.

51. A method of detecting the presence or absence of fecal matter in a sample, said method comprising assessing the presence or absence of resistin-like molecule β in a sample, wherein the presence of resistin-like molecule β in said sample is an indication that fecal matter is present in said sample, and wherein the absence of resistin-like molecule β in said sample is an indication that fecal matter is absent from said sample, thereby detecting the presence or absence of fecal matter in a sample.

52. The method of claim 51, wherein said assessing comprises contacting said sample with an antibody that specifically binds with resistin-like molecule β and detecting binding of said antibody with resistin-like molecule β in said sample.

53. A kit for alleviating a disease mediated by malexpression of a resistin-like molecule in a human, said kit comprising a resistin-like molecule expression-inhibiting amount of a composition comprising an isolated nucleic acid complementary to an isolated nucleic acid encoding a mammalian resistin-like molecule, or a fragment thereof, said complementary nucleic acid being in an antisense orientation, and a pharmaceutically-acceptable carrier, said kit further comprising an applicator, and an instructional material for the use thereof.

54. The kit of claim 53, wherein said disease is selected from the group consisting of irritable bowel disease, inflammatory bowel disease, familial adenomatous polyposis, an intestinal tumor, diabetes, insulin resistance, obesity, breast cancer, tongue cancer, and colon cancer.

55. A kit for alleviating a disease mediated by malexpression of a resistin-like molecule in a human, said kit comprising a resistin-like molecule expression-inhibiting amount of a composition comprising an antibody that specifically binds with a mammalian resistin-like molecule polypeptide, or a fragment thereof, and a pharmaceutically-acceptable carrier, said kit further comprising an applicator, and an instructional material for the use thereof.
